(19) United States
(12) Patent Application Publication
(10) Pub. No.: US 2024/0018009 A1
(43) Pub. Date: Jan. 18, 2024

(54) PARTICLES COMPRISING AMORPHOUS DIVALENT METAL CARBONATE

(71) Applicant: AMORPHICAL LTD., Ness Ziona (IL)

(72) Inventors: Yossi BEN, Moshav Zofer (IL); Yigal BLUM, San Jose, CA (US); Roni Antoine ILLOS, Ashkelon (IL); Ben ASHKENAZI, Ofakim (IL)

(21) Appl. No.: 18/227,077

(22) Filed: Jul. 27, 2023

Related U.S. Application Data
(63) Continuation of application No. PCT/IL2022/050122, filed on Jan. 27, 2022.
(60) Provisional application No. 63/142,631, filed on Jan. 28, 2021.
(60) Provisional application No. 63/243,770, filed on Sep. 14, 2021.

(51) Int. Cl. C01F 5/24; C01F 11/18; C01G 9/00; A61K 33/10; A61K 33/30; A61K 33/42; A61K 9/16; A61K 9/10

(52) U.S. Cl. CPC C01F 5/24; C01F 11/18; C01G 9/00; A61K 33/10; A61K 33/30; A61K 33/42; C01P 2002/02; A61K 9/10; C01P 2004/03; C01P 2004/62; C01P 2004/64; C01P 2006/12; A61K 9/16

(57) ABSTRACT

The present invention provides a composition including a particle, the particle includes a carbonate species of a first divalent metal and carbonate specie of a second divalent metal; wherein the particle is amorphous; wherein the first divalent metal and the second divalent metal are in an oxidation state of +2; and wherein the first divalent metal and the second divalent metal is each independently selected from: Ca, Zn, Fe, Cu, Co, Ni, Mn, Mg and Cr. Further provided is a composition including a divalent metal carbonate including any derivative thereof, and at least one stabilizing agent, wherein the divalent metal is selected from: Zn, Cu, Co, Mn, Mg, and Ni, optionally being characterized by BET between 20 and 200 m²/g. Pharmaceutical or nutraceutical compositions including the composition of particles disclosed, are also provided.

### CROSS-REFERENCE TO RELATED APPLICATIONS

This application is a bypass Continuation of PCT Patent Application No. PCT/IL2022/050122 having International filing date of Jan. 27, 2022, which claims the benefit of priority of U.S. Provisional Patent Application No. 63/142,631, titled “PARTICLES COMPRISING AMORPHOUS DIVALENT METAL CARBONATE”, filed Jan. 28, 2021, and of U.S. Provisional Patent Application No. 63/243,770, titled “PARTICLES COMPRISING AMORPHOUS DIVALENT METAL CARBONATE”, filed Sep. 14, 2021. The contents of the above referenced applications are all incorporated herein by reference in their entirety.

### FIELD OF THE INVENTION

The present invention relates generally to the field of nanoparticles comprising amorphous divalent metal carbonate.

### BACKGROUND

Local or systemic acidosis conditions (pH<7.35) are direct and harmful outcomes of a broad range of severe diseases and body energetic stresses, and they enhance the disease progression and body stress. The ability to neutralize the excessive acidity in a safely and timely fashion, “just in time”, in a controlled fashion, without overreacting such a basic agent to generate the undesired alkalosis conditions (pH>7.45) is highly desired as acidosis therapy. Solid nanoparticles of divalent metal carbonates, and to a certain extent, higher valency metal carbonates, mainly in homogeneous association with divalent metal carbonates can provide this desired capability if their solubility in the range of pH 6.5 to 7.35 is sufficient and if the nanoparticles can be delivered directly to the blood or other body fluids or by fusion through mucous membranes.

Some non-alkali metal ions are highly or mildly required as nutrients needed by biological species, including Ca, Mg, Fe, Zn, Cr, Cu, Co, Mn, and Ni. The latter ones' primary function is their participation as co-factors at the activity center of enzymes, and therefore are necessary at trace levels well below 1% of the overall metal content. Such metals can be absorbed better if they are not easily washed away by the body liquids and if they are slowly release. Nanometric particles of such metal carbonates and combinations thereof can be transfused into the blood system in their nanometric morphology and only then being slowly released for maximizing the utilization of the metal ions.

Additional benefits will be if controlling the solubility rates and final pH of the affected area can be achieved. Such activities will allow slow or fast release, targeted release, and controlled release per condition needs. Carbonate ions can serve as very efficient neutralization agents converting mainly to the very bio essential bicarbonate ion or to a lesser extent CO₂ in the pH range of acidosis. Hence, the neutralization products are very useful for the body functioning by themselves or be incorporated and removed by the regular respiration or renal body functions.

The use of the carbonate anions was considered for their therapeutic activity only at the stomach metabolism state. There was no consideration for utilizing carbonates of divalent metals as a solid but efficient source for delivering carbonate ions to other parts of the body that possess extracellular acidity level below the normal of pH<7.35, i.e., acidosis conditions.

The dissolution rates of such divalent metal carbonates can be tailored based on the selected metal or combination of metals that constitute the carbonate, its crystallinity stage and phase, and a variety of incorporated stabilizers, if the preferred phase of the desired divalent carbonate is amorphous.

The use of the carbonate anions was considered for their therapeutic activity only at the stomach metabolism state. There was no consideration for utilizing carbonates of divalent metals as a solid but efficient source for delivering carbonate ions to other parts of the body that possess extracellular acidity level below the normal of pH<7.35, i.e., acidosis conditions.

The dissolution rates of such divalent metal carbonates can be tailored based on the selected metal or combination of metals that constitute the carbonate, its crystallinity stage and phase, and a variety of incorporated stabilizers, if the preferred phase of the desired divalent carbonate is amorphous.

Divalent metal atoms such as Fe, and Zn play an important role in various biological systems of the human body, including functions at the cell, protein, and biochemical levels. Therefore, it will be advantageous to have the capability of transferring them to the targeted tissues and body systems as nanometric divalent metal and mixed metals carbonates, that can unload both their metallic and carbonate entities in a slow-release fashion.

Amorphous and nanometric carbonates of divinyl metal and mixed divinyl metals are typically unstable and tend to crystalize rapidly, especially when placed in suspensions or exposed to moisture and elevated temperature. The synthesis, processing, and storage, especially in the form of suspensions of divalent metal carbonates was found to be a significant challenge. The crystallization is primarily fast in the presence of water and moisture. Hence, stabilizing such desired nanometric compositions is desired.

The foregoing examples of the related art and limitations related therewith are intended to be illustrative and not exclusive. Other limitations of the related art will become apparent to those of skill in the art upon a reading of the specification.

### SUMMARY

According to a first aspect, there is provided a composition comprising a particle, the particle comprising a carbonate species of a first divalent metal and carbonate specie of a second divalent metal; the particle has an average diameter of between 5 and 500 nm, or between 5 and 200 nm, and at least 70% by weight of the composition is amorphous; wherein: any one of the first divalent metal and the second divalent metal is selected from the group consisting of: Ca, Zn, Fe, Cu, Co, Ni, Mn, and Cr; and a molar ratio between the first divalent metal and the second divalent metal is between 10:0.1 and 3:1.

According to another aspect, there is provided a composition, comprising a divalent metal carbonate including any derivative thereof, and a at least one stabilizing agent, wherein the particle has a diameter of between 5 and 500 nm, or between 5 and 200 nm, and at least 70% by weight of the divalent metal carbonate is amorphous; wherein: the divalent metal is selected from the group consisting of: Zn, Cu, Co, Ni, and Mn; and a molar ratio between the divalent metal and the at least one stabilizing agent is between 100:1 and 100:25.

According to another aspect, there is provided a composition comprising a plurality of particles disclosed herein, and an acceptable carrier.

In some embodiments, the particle further comprises a stabilizer.

In some embodiments, the molar ratio between the stabilizer and the first divalent metal within the particle is between 1:100 and 25:100.

In some embodiments, the stabilizer is selected form the group consisting of: polyphosphate, phosphoserine, adenosine triphosphate, adenosine diphosphate, phytic acid, citric acid, etidronic acid, pyrophosphate, ethanol, and any combination thereof.

In some embodiments, the polyphosphate is selected form the group consisting of: diphosphate, triphosphate, trimetaphosphate, hexametaphosphate, and any combination thereof.

In some embodiments, the water content of the composition is between 1 and 30% by weight.

In some embodiments, the w/w concentration of the first divalent metal within the particle is between 20 and 40%.

In some embodiments, the particle is stable for at least 24 h under appropriate conditions.

In some embodiments, the divalent metal carbonate or a hydrate thereof is Zn.

In some embodiments, the weight content of the divalent metal within the particle is between 20 and 60%.

In some embodiments, at least a portion of the plurality of particles is in a form of an agglomerate or an aggregate.

In some embodiments, the agglomerate or aggregate is characterized by having a mean diameter of between 0.1 and 10 μm.

In some embodiments, the composition is in a form of (i) a solid composition or (ii) a suspension.

In some embodiments, the composition is a pharmaceutical or a nutraceutical composition.

According to another aspect, there is provided a composition, comprising a plurality of particles, each of the plurality of particles comprises a divalent metal carbonate including any derivative thereof, and a at least one stabilizing agent, wherein the particle has a diameter of between 5 and 500 nm, and at least 70% by weight of the divalent metal carbonate is amorphous; wherein the divalent metal is selected from the group consisting of: Zn, Cu, Co, Ni, Mn, and Mg; and a molar ratio between the divalent metal and the at least one stabilizing agent is between 100:1 and 100:25.

In some embodiments, the divalent metal is Mg, then the plurality of particles are characterized by a BET surface area of at most 200 m²/g.

In some embodiments, the stabilizer is selected from the group consisting of: polyphosphate, phosphoserine, adenosine triphosphate, adenosine diphosphate, phytic acid, citric acid, etidronic acid, pyrophosphate, ethanol, and any combination thereof.

In some embodiments, the polyphosphate is selected form the group consisting of: diphosphate, triphosphate, trimetaphosphate, hexametaphosphate, and any combination thereof.

In some embodiments, the divalent metal carbonate or a hydrate thereof is Zn or Mg.

In some embodiments, a weight content of the divalent metal within the particle is between 20 and 60%.

In some embodiments, at least 90%, or at least 95% by weight of the divalent metal carbonate within the composition is amorphous.

In some embodiments, the composition further comprising a pharmaceutically acceptable carrier.

In some embodiments, the pharmaceutically acceptable carrier is a liquid carrier and wherein the composition is in a form of a suspension.

In some embodiments, the suspension is substantially stable for at least 1 h.

In some embodiments, the composition comprises the particles of the invention and a pharmaceutically acceptable liquid carrier, wherein the divalent metal is selected from the group consisting of: Zn, and Mg.

In some embodiments, at least about 80% by total weight of the divalent metal carbonate remains amorphous within the liquid composition for a time period of at least 1 d.

In some embodiments, the composition is in a form of a powder, and wherein at least 95% by total weight of the divalent metal carbonate remains amorphous upon storage of the composition for at least 1 month under appropriate conditions.

In some embodiments, a water content of the powder is between 1 and 30% by weight.

According to another aspect, there is provided a composition comprising a plurality of particles, each of the plurality of particles comprises a first divalent metal, a second divalent metal, a carbonate specie and a stabilizer; the particle has a particle size of between 5 and 500 nm, and at least 70% by total weight of the carbonate species within the composition are amorphous; wherein the first divalent metal is selected from the group consisting of: Ca, Zn, Fe, Cu, Co, Ni, Mn, and Cr; the second divalent metal is Mg; and a molar ratio between the first divalent metal and the second divalent metal is between 10:0.0.05 and 10.0:9.9.

In some embodiments, a molar ratio between the stabilizer and the first divalent metal within the particle is between 1:100 and 25:100.

In some embodiments, the stabilizer is selected form the group consisting of: polyphosphate, phosphoserine, adenosine triphosphate, adenosine diphosphate, phytic acid, citric acid, etidronic acid, pyrophosphate, ethanol, and any combination thereof.

In some embodiments, the polyphosphate is selected form the group consisting of: diphosphate, triphosphate, trimetaphosphate, hexametaphosphate, and any combination thereof.

In some embodiments, a w/w concentration of the first divalent metal within the particle is between 20 and 55%.

In some embodiments, a molar ratio between the first divalent metal and Mg is between 10:0.05 and 1:1, or between 10:0.05 and 10:3.

In some embodiments, the composition further comprises a pharmaceutically acceptable carrier.

In some embodiments, the pharmaceutically acceptable carrier is a liquid carrier and wherein the composition is in a form of a suspension.

In some embodiments, the suspension is substantially stable for at least 1 h.

In some embodiments, at least about 80% by total weight of the carbonate specie remains amorphous within the composition for a time period of at least 1 d.

In some embodiments, a water content of the composition is between 1 and 30% by weight.

In some embodiments, the composition is in a form of a powder, and wherein at least 95% by total weight of the carbonate specie remains amorphous upon storage of the composition for at least 1 month under appropriate conditions.

In some embodiments, the particles are characterized by a BET surface area of between 20 and 200 m²/g.

In some embodiments, the stabilizer is substantially homogenously distributed within the particle.

In some embodiments, the divalent metal is in a form of a divalent metal salt, hydroxide, hydroxyl, oxide, a hydrate or any combination thereof.

In some embodiments, at least a portion of the plurality of particles is in a form of an agglomerate or an aggregate.

In some embodiments, the agglomerate or aggregate is characterized by having an average particle size of between 0.1 and 200 μm.

In some embodiments, a molar ratio of the carbonate specie to a total divalent metal content of the particles is between 0.1:1 and 1:1.

In some embodiments, the carbonate specie is in a form of (i) a metal carbonate of the first divalent metal, (ii) a metal carbonate of the second divalent metal or both (i) and (ii).

In some embodiments, the composition of the invention is a pharmaceutical or a nutraceutical composition.

In some embodiments, the composition of the invention comprises a therapeutical effective amount or a nutraceutical effective amount of the particles.

In addition to the exemplary aspects and embodiments described above, further aspects and embodiments will become apparent by study of the following detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 includes a micrograph representing SEM images of aggregated amorphous metal carbonate nanoparticles including ACC, AMC, AZC and meta-ACCs consisting of agglomerated primary nanoparticles in the range of 10 to 100 nm.

FIG. 2 includes a graph representing TGA curves of exemplary compositions of the invention. M-ACC-TP reveals 4 significant regions of weight loss at the following maximum rate temperature: ˜90° C. (loss of adsorbed water); ˜240° C. (loss of complexed and trapped water); ˜400° C. (water due to condensation of —OH groups); ˜680° C. (CO₂ due to carbonate decomposition).

FIG. 3 includes a graph representing XRD patterns of exemplary amorphous metal carbonates of the invention and Metal-included ACC.

FIG. 4 includes graphs representing Solid State ¹³C NMR analysis of exemplary amorphous metal carbonates of the invention.

FIG. 5 includes a graph representing a carbonate fraction (molar ratio relative to calcium) of the exemplary composition of the invention, as a function of triphosphate stabilizer (molar ratio relative to calcium) added during the synthesis.

FIG. 6 includes a graph showing the incorporation of phosphoserine (PS) stabilizer into the entire ACC particle and/or adsorption to the outer surface of the particle, as a function of Ca/PS molar ratio.

FIG. 7 includes a graph showing the incorporation of tripolyphosphate (TPP) stabilizer into the entire ACC particle and/or adsorption to the outer surface of the particle, as a function of Ca/TPP molar ratio.

FIG. 8 includes a graph showing protein concentration. A549 cells were grown for 3 passages in different treatments. After 3 passages cells were subjected to cathepsin B assay, in triplicates. 50 μl from each treatment were taken for the assay. AFC (7-Amino-4-trifluoromethylcoumarin), Fluorescent marker is used to create standard curve for Cathepsin B activity (converting O.D=Optical density into activity). ****P value<0.0001.

FIG. 9 includes a graph showing protein concentration presented as fold change from bank. A549 cells were grown for 3 passages in different treatments. After 3 passages cells were subjected to cathepsin B assay, in triplicates. 50 μl from each treatment were taken for the assay. Ordinary one-way ANOVA, ****P value<0.0001.

### DETAILED DESCRIPTION

According to one aspect, there is provided a composition comprising a plurality of particles, each particle comprises a carbonate specie comprising a first divalent metal and a second divalent metal; wherein at least 70% by weight of the carbonate specie within the composition is amorphous; and wherein any one of the first divalent metal and the second divalent metal is selected from: Ca, Zn, Fe, Cu, Co, Ni, Mn, or Cr. In some embodiments, the second divalent metal comprises one or more metals.

According to another aspect, there is provided a composition comprising a plurality of particles, each particle comprises a carbonate specie comprising a first metal and a second metal; wherein at least 70%, or at least 99% by weight of the carbonate specie within the composition is amorphous; and wherein any one of the first metal and the second metal is selected from: Ca, Zn, Fe, Cu, Co, Ni, Mn, or Cr.

According to another aspect, there is provided a composition comprising a plurality of particles, each particle comprises a carbonate specie comprising a first metal and a second metal, and further comprises a stabilizer; wherein at least 70%, or at least 99% by weight of the carbonate specie within the composition is amorphous; and wherein any one of the first divalent metal and the second divalent metal is selected from: Ca, Zn, Fe, Cu, Co, Ni, Mn, Mg, or Cr, and wherein the plurality of particles is characterized by BET surface area of at most 500 m²/g, or at most 200 m²/g.

### Particle

In some embodiments, the particle of the invention is a nanoparticle having an average particle size between 5 and 200 nm. In some embodiments, the particle of the invention is a nanoparticle having an average particle size between 5 and 900 nm. In some embodiments, the particle of the invention comprises a divalent metal carbonate specie. In some embodiments, the divalent metal carbonate specie comprises a carbonate group and a divalent metal. In some embodiments, the particle of the invention comprises a single metal carbonate specie. In some embodiments, the particle of the invention comprises a single divalent metal specie. In some embodiments, the particle of the invention comprises a single divalent metal carbonate specie, wherein at least 70%, at least 75%, at least 80%, at least 85%, at least 90%, at least 95%, or at least 99% by weight of the carbonate specie within the composition is amorphous, including any range between. In some embodiments, the particle of the invention is an amorphous metal carbonate particle. In some embodiments, the particle of the invention is a nanoparticle. In some embodiments, the particle of the invention is an amorphous metal carbonate nanoparticle, wherein the metal comprises a divalent metal, a trivalent metal, or a tetravalent metal.

In some embodiments, the metal (e.g., divalent metal) comprises a plurality of chemically different metal species. In some embodiments, the divalent metal comprises a first metal atom and a second metal atom, wherein the first metal atom and the second metal atom are independently selected from: Ca, Mg, Zn, Fe, Cu, Co, Ni, Mn, or Cr, wherein if the first metal atom is Ca, then the second metal atom is selected from the: Zn, Fe, Cu, Co, Ni, Mn, Cr, or any combination thereof.

In some embodiments, the terms “divalent metal carbonate specie” and “carbonate specie” are used herein interchangeably.

In some embodiments, the divalent metal carbonate specie comprises a single divalent metal selected from: Zn, Fe, Cu, Co, Ni, Mn, or Cr. In some embodiments, the divalent metal carbonate specie comprises a plurality of divalent metals, wherein the plurality of divalent metals comprises a first metal atom and a second metal atom, wherein the first metal atom and the second metal atom are independently selected from: Ca, Mg, Zn, Fe, Cu, Co, Ni, Mn, or Cr, wherein if the first metal atom is Ca, then the second metal atom is selected from: Zn, Fe, Cu, Co, Ni, Mn, Cr, or any combination thereof.

In some embodiments, the divalent metal carbonate specie of the invention is as described herein. In some embodiments, the amorphous metal carbonate species of the invention or the amorphous multi metal carbonates are further stabilized with an additional stabilizer.

In some embodiments, the particle of the invention further comprises an additional metal atom (e.g., residual monovalent metal atom, such as Na, Li, K and Sr or higher valent metal, e.g., Cr (III), Mn (IV), Fe (III) Co (III) and Ni (IV). In some embodiments, the additional metal atom is a non-active ingredient. In some embodiments, the additional metal atom is present at trace amounts within the particle of the invention.

In some embodiments, the particle of the invention is substantially devoid of an additional metal (such as monovalent metal, divalent metal, trivalent metal, or any combination thereof). In some embodiments, the particle of the invention comprises trace amounts of monovalent metals including any salt thereof, such as sodium carbonate, sodium polyphosphates, and sodium phosphate.

As used herein, the term “divalent metal” refers to a metal in an oxidation state (II). As used herein, the term “monovalent metal” refers to a metal in an oxidation state (I). Higher valent metals can be “trivalent” and “tetravalent” metals. In some embodiments, the divalent metal of the invention consists essentially of metals in an oxidation state of II, wherein the metal is selected from the elements as described herein.

In some embodiments, at least 70%, 80%, at least 85%, at least 87%, at least 90%, at least 92%, at least 95%, at least 97%, at least 98%, at least 99% by weight of the metal content of the particle of the invention is composed of one or more divalent metal(s), wherein the one or more divalent metal(s) is as described herein.

In some embodiments, at least 70%, 80%, at least 85%, at least 87%, at least 90%, at least 92%, at least 95%, at least 97%, at least 98%, or at least 99% by weight of the metal content of composition of the invention is composed of one or more divalent metal(s), wherein the one or more divalent metal(s) is as described herein.

In some embodiments, the particle of the invention further comprises a stabilizer. The terms “stabilizing agent” and “stabilizer” are used herein interchangeably and refer to any substance (e.g., and organic or an inorganic molecule, and/or a salt thereof) that contributes to preserving the divalent metal carbonate within the particle in the amorphous state during production, formulating, storage and/or use. In certain embodiments, the stabilizing agent is a single agent. In other embodiments, use of several stabilizing agents is encompassed. In some embodiments the stabilizer is an organic or inorganic oligomer or polymer.

In some embodiments, the carbonate specie of the invention is in an amorphous state, stabilized by at least one stabilizing agent, wherein amorphous is as described hereinbelow. In some embodiments, the divalent metal carbonate specie of the invention is in an amorphous state, at least partially stabilized by the second divalent metal specie.

The stabilizer may comprise a molecule having one or more functional groups selected from, but not limited to, hydroxyl, carboxyl, ester, amine, phosphino, phosphono, phosphate, sulfonyl, sulfate or sulfino groups. The hydroxy bearing compounds, combined with the hydroxide, optionally also bear other functional groups like carboxyl, etc. but with the hydroxyl not being esterified.

According to some embodiments, the stabilizer has low toxicity or no toxicity to mammalian cells or organism, and in particular to a human being. According to some embodiments, the stabilizer is of food, nutraceutical or pharmaceutical grade. In some embodiments, the stabilizer is selected from the group consisting of organic acids, phosphorylated, phosphonated, sulfated or sulfonated organic compound, phosphoric or sulfuric esters of hydroxy carboxylic acids, glucose and its derivatives, polysaccharides, phosphorylated amino acids, bisphosphonate, polyphosphonates, organic polyphosphate, inorganic polyphosphates, hydroxyl bearing organic compounds and polyols, proteins, derivatives thereof, divalent metal atom (e.g., Mg, Mn, Zn, Fe), and any combination thereof.

In some embodiments, the stabilizer is selected from the group consisting of polyphosphate, phosphoserine, adenosine triphosphate, adenosine diphosphate, phytic acid, citric acid, a dicarboxylic acid, a tricarboxylic acid, etidronic acid, pyrophosphate, ethanol, and any combination thereof.

In some embodiments, the stabilizer is independently at each occurrence, an 10 organic acid, phosphorylated, phosphonated, sulfated or sulfonated organic compound, phosphoric or sulfuric ester of a hydroxyl carboxylic acid, an organoamine compound, an organic compound comprising a hydroxyl, an organophosphorus compound or a salt thereof, phosphorylated amino acids and derivatives thereof, a bisphosphonate compound, an organophosphate compound, an organophosphonate compound, an inorganic phosphorous acid, an organic compound having multiple functional groups as defined above, an inorganic phosphate and polyphosphate compound, an organic compound having a polyphosphate chain, an organic surfactant, a bio-essential inorganic ion, or any combination thereof.

In some embodiments, the stabilizer is an organic acid. In some embodiments, the organic acid is selected from ascorbic acid, citric acid, lactic acid, acetic acid, oxalic acid, malonic acid, glutaconic acid, succinic acid, maleic acid, lactic acid, aconitic acid, and optionally include compounds having at least two carboxylic groups and molecular weight not larger than 250 g/mol, such as citric acid, tartaric acid, malic acid, etc. In some embodiments, the stabilizer is citric acid.

In some embodiments, the phosphoric ester of hydroxyl carboxylic acids is a phosphoenolpyruvate. In some embodiments, the phosphoric or sulfuric esters of hydroxyl carboxylic acids comprise amino acids, e.g., phosphorylated amino acids. Examples of such esters are phosphoserine, phosphothreonine, sulfoserine, sulfothreonine and phosphocreatine.

In some embodiments, the hydroxyl bearing compounds combined with hydroxide comprise, for example, mono-, di- tri-, oligo-, and polysaccharides like sucrose or other polyols like glycerol. In some embodiments, the hydroxyl bearing compounds may further comprise hydroxy acids like citric acid, tartaric acid, malic acid, etc., or hydroxyl-bearing amino acids such as serine or threonine. Each possibility represents a separate embodiment, of the present invention.

Some specific unlimited examples of stabilizers include phytic acid, citric acid, sodium pyrophosphate dibasic, adenosine 5′-monophosphate (AMP) sodium salt, adenosine 5 5′-diphosphate (ADP) sodium salt and adenosine 5′-triphosphate (ATP) disodium salt hydrate, phosphoserine, phosphorylated amino acids, food grade surfactants, sodium stearoyl lactylate, and combinations thereof.

In some embodiments, the stabilizer comprises at least one component selected from phosphoric or sulfuric esters of hydroxyl carboxylic acids, such as phosphoenolpyruvate, phosphoserine, phosphorthreonine, sulfoserine or sulfothreonine and hydroxyl bearing organic compounds, selected from mono-, di-, tri-, oligo- and polysaccharides, for example, sucrose, mannose, glucose. The hydroxyl bearing compound may further comprise at least one alkali hydroxide, such as sodium hydroxide, potassium hydroxide and the like. The phosphorylated acids may be present in oligopeptides and polypeptides. In some embodiments, the stabilizer is an organic acid selected from monocarboxylic acid or multiple carboxylic acid, e.g., dicarboxylic acid or tricarboxylic acid. Each possibility represents a separate embodiment, of the invention. In some embodiments, the organic acid is as described above.

In some embodiments, the stabilizer is selected from phosphorylated amino acids, polyols and combinations thereof. In some embodiments, the divalent metal carbonate specie is stabilized by a phosphorylated compound, wherein the phosphorylation is performed on the hydroxyl group of an organic compound. In some embodiments, the divalent metal carbonate specie is stabilized by a compound selected from: citric acid, phosphoserine, phosphothreonine or any combinations thereof. The non-limiting examples of stabilizers containing phosphate, phosphite, phosphonate groups and salts or esters thereof include phytic acid, dimethyl phosphate, trimethyl phosphate, sodium pyrophosphate, tetraethyl pyrophosphate, ribulose bisphosphate, etidronic acid and other medical bisphosphonates, 3-phosphoglyceric acid salt, glyceraldehyde 3-phosphate, 1-deoxy-D-xylulose-5-phosphate sodium salt, diethylene triamine pentakis(methylphosphonic acid), 30 nitrilotri(methylphosphonic acid), 5-phospho-D-ribose 1-diphosphate pentasodium salt, adenosine 5′-diphosphate sodium salt, adenosine 5′-triphosphate disodium salt hydrate, α-D-galactosamine 1-phosphate, 2-phospho-L-ascorbic acid trisodium salt, α-D-galactose 1-phosphate dipotassium salt pentahydrate, α-D-galactosamine 1-phosphate, 0-phosphorylethanolamine, disodium salt hydrate, 2,3-diphospho-D-glyceric acid pentasodium salt, phospho(enol)pyruvic acid monosodium salt hydrate, D-glyceraldehyde 3-phosphate, sn-glycerol 3-phosphate lithium salt, D-(−)-3-phosphoglyceric acid disodium salt, D-glucose 6-phosphate sodium salt, phosphatidic acid, ibandronate sodium salt, 5 phosphonoacetic acid, DL-2-amino-3-phosphonopropionic acid or combinations thereof. The bio-essential inorganic ions may include, inter alia, Na, K, Mg, Zn, Fe, P, S, N, P or S in the phase of oxides, or N as ammonia or nitro groups.

In some embodiments, the stabilizer is a polyphosphate or a pharmaceutically acceptable salt thereof. In some embodiments, the polyphosphate is physiologically compatible, water-soluble polyphosphate salt selected from the group consisting of sodium, potassium, and any other essential salt of polyphosphate. In some embodiments, the polyphosphate is organic or inorganic polyphosphate. The term “polyphosphate” as used herein refers to polymeric esters of phosphate (PO₄³⁻). In some embodiments, the polyphosphate is physiologically compatible water-soluble polyphosphate salt selected from the group consisting of sodium and potassium polyphosphate. In some embodiments, the polyphosphate is an inorganic polyphosphate or pharmaceutically acceptable salts thereof.

Not-limiting examples of such salts are Na, K, and NH₄ cations. According to some embodiments, the inorganic phosphate comprises 2 to 10 phosphate groups, e.g., 2, 3, 4, 5, 6, 7, 8, 9, or 10 phosphate group. In some embodiments, the polyphosphate is selected from pyrophosphate, triphosphate, and hexametaphosphate. In some embodiments, the stabilizer is pyrophosphate or pharmaceutically acceptable salts thereof such as sodium pyrophosphate. In some embodiments, the stabilizer is triphosphate or pharmaceutically acceptable salts thereof such as sodium triphosphate. The term “triphosphate” and “tripolyphosphate” are used herein interchangeably. In some embodiments, the stabilizer is hexametaphosphate or a pharmaceutically acceptable salt thereof such sodium hexametaphosphate.

In some embodiments, the stabilizer is a bisphosphonate or pharmaceutically acceptable salts thereof. The not-limiting examples of such salts are Na, K, and NH₄ cations.

The term “bisphosphonate” as used herein refers to organic compounds having two phosphonate (PO(OH)₂) groups. The term further relates to compounds having a backbone of PO₃-organic-PO₃. Most typical is a series of bisphosphonates that are used as pharmaceuticals for treating osteoporosis. In some embodiments, the bisphosphonate is selected from the group consisting of etidronic acid, zoledronic acid, medronic acid, alendronic acid and a pharmaceutically acceptable salt thereof. In some embodiments, stabilizer is an etidronic acid or a pharmaceutically acceptable salt thereof. In some embodiments, stabilizer is a zoledronic acid or a pharmaceutically acceptable salt thereof. In some embodiments, stabilizer is a medronic acid or a pharmaceutically acceptable salt thereof. In some embodiments, stabilizer is alendronic acid or a pharmaceutically acceptable salt thereof.

In some embodiments, stabilizer is a phosphorylated amino acid. In some embodiments, phosphorylated amino acid is phosphoserine. In some embodiments, phosphorylated amino acid is phosphothreonine.

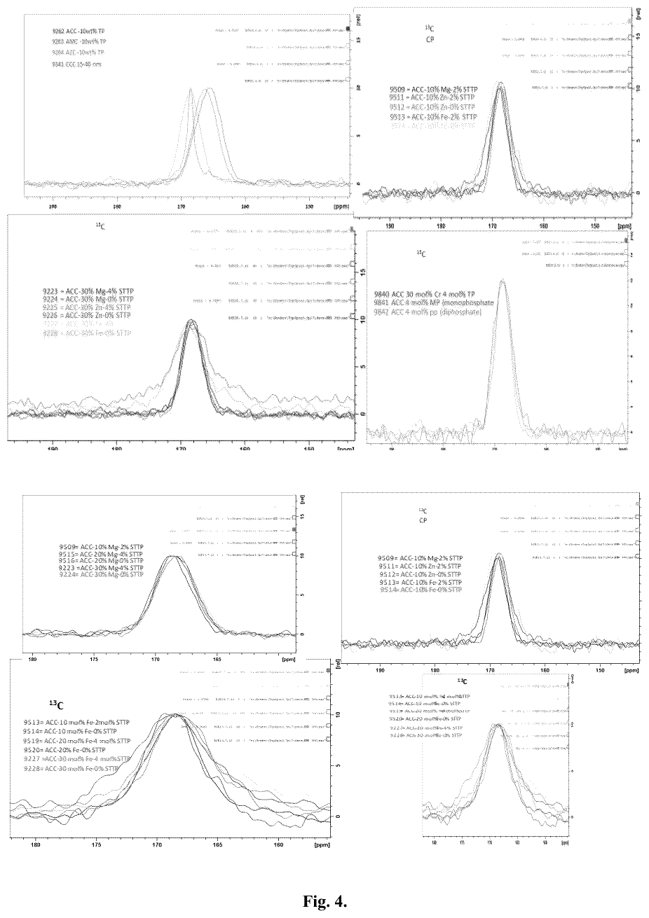

In some embodiments, the divalent metal carbonate specie of the invention is in an amorphous state (also used herein as “amorphous carbonate specie”), stabilized by more than one stabilizers, e.g., two or more stabilizers. In some embodiments, any of the particles of the invention and/or a composition comprising thereof comprises a single amorphous carbonate specie. In some embodiments, the amorphous carbonate species within the particle and/or composition of the invention are chemically identical (based on Solid State NMR data, as exemplified in FIG. 4).

In some embodiments, more than one stabilizers, e.g., 2, 3 or 4 chemically distinct stabilizer species are included. In some embodiments, the particle comprises a first stabilizer and a second stabilizer. In some embodiments, the first stabilizer and the second stabilizer are the same. In some embodiments, the first stabilizer and the second stabilizer are different stabilizers. In some embodiments, the first and the second stabilizers are each independently as described hereinabove. In some embodiments, the amorphous carbonate specie comprises more than two stabilizers, wherein one or more stabilizers are added thereto during the formation and precipitation of the particle of the invention.

In some embodiments, the amorphous carbonate specie is stabilized by a combination of phosphoserine and citric acid. In some embodiments, the amorphous carbonate specie is stabilized by a combination of triphosphate and citric acid.

In some embodiments, the amorphous carbonate specie is stabilized by triphosphate.

In some embodiments, at least a portion of the divalent metal carbonate specie (e.g., amorphous carbonate specie) is bound to the stabilizer. In some embodiments, the divalent metal carbonate specie and the stabilizer are bound via a covalent and/or a non-covalent bond, as described herein. In some embodiments, the stabilizer is incorporated into the divalent metal carbonate specie (e.g., amorphous carbonate specie). In some embodiments, the stabilizer is uniformly distributed within the divalent metal carbonate specie (e.g., amorphous carbonate specie).

In some embodiments, the stabilizer is a phosphate-based stabilizer (such as mono-, di- or triphosphate, polyphosphate, or a bisphosphonate as defined hereinabove), and the molar ratio between P atoms of the stabilizer and the divalent metal atom (e.g., first divalent metal) within the particle (P:M molar ratio) is between 1:100 to 50:100, between 1:100 to 3:100, between 3:100 to 5:100, between 5:100 to between 10:100 to 15:100, between 15:100 to 20:100, between 20:100 to between 30:100 to 40:100, including any range or value therebetween. Each possibility represents a separate embodiment of the invention.

In some embodiments, the P:M molar ratio is between 4:100 to 15:100, between 4:100 to 8:100, between 8:100 to 10:100, between 10:100 to 12:100, between 12:100 to 15:100, including any range or value therebetween. Each possibility represents a separate embodiment of the invention.

In some embodiments, a molar ratio between the stabilizer and divalent metal atom (e.g., first divalent metal) within the particle (e.g. a single metal carbonate-based particle) is between 1:100 to 15:100, between 1:100 to 3:100, between 3:100 to 5:100, between 5:100 to 8:100, between 8:100 to 10:100, between 10:100 to 12:100, between 12:100 to 15:100, between 15:100 to 20:100, between 20:100 to 25:100, between to 30:100 including any range or value therebetween. In some embodiments, a molar ratio between the stabilizer and the divalent metal atom is at least 0.5%, at least at least 1%, at least 1.1%, at least 1.5%, at least 2%, at least 4%, at least 8% including any range or value therebetween. In some embodiments, a weight ratio between the stabilizer and divalent metal atom is at least 5%, at least 8%, at least 10%, at least 20% including any range or value therebetween. Each possibility represents a separate embodiment of the invention. Without being limited to any particular theory, it is postulated that at least 8%, or at least 10% by weight of the stabilizer (relative to the metal) is required for obtaining a stable particle of the invention (e.g. a single-metal particle), wherein the term “stable” is as described herein (e.g. refers to the ability of the dry particles and/or the powderous composition comprising thereof to retain at least 80%, or at least 95% by total weight of the divalent metal carbonate (also including mixed metal carbonates) in an amorphous state for at least 1 month under appropriate conditions).

In some embodiments, the stabilizer is or comprises triphosphate including any salt thereof.

In some embodiments, the stabilizer is polyphosphate, such as pyrophosphate, triphosphate, hexametaphosphate or a pharmaceutically acceptable salt thereof, wherein the P:M molar ratio is as described herein (e.g., between 4:100 and 15:100). In some embodiments, the stabilizer is bisphosphonate, such as alendronic acid, etidronic acid, zoledronic acid or medronic acid, wherein the P:M molar ratio is as described herein (e.g., between 4:100 and 15:100).

In some embodiments, at least 80%, at least 85%, at least 87%, at least 90%, at least 92%, at least 95%, at least 97%, at least 98%, or at least 99% by weight of the stabilizer of the invention is as described hereinabove, or any value and range therebetween. Each possibility represents a separate embodiment of the invention.

In some embodiments, the particle or the nanoparticle of the invention (also used herein as “primary particle”) has a mean size (e.g., mean diameter) of: between 5 and 900 nm, between 5 and 900 nm, between 5 and 10 nm, between 10 and 50 nm, between 20 and 80 nm, between 20 and 150 nm, between 50 and 100 nm, between 100 and 200 nm, between 200 and 300 nm, between 300 and 400 nm, between 400 and 500 nm, between 500 and 700 nm, between 700 and 900 nm, including any range or value therebetween. In some embodiments, the particle, or the nanoparticle of the invention (also used herein as “primary particle”) has a mean size (e.g., mean diameter) of: between 5 and 200 nm, between 5 and 200 nm, between 5 and 10 nm, between 10 and 50 nm, between 20 and 80 nm, between 20 and 150 nm, between 50 and 100 nm, between 100 and 200 nm, including any range or value therebetween. Each possibility represents a separate embodiment of the invention. In some embodiments, the term “mean size” and the term “average particle size” are used herein interchangeably.

As used herein the term “primary” refers to basic particle formed during the synthesis, which can be further fused, agglomerated, and/or aggregated into larger particles and networks of particles (e.g., a secondary particle).

As used herein the terms “diameter” or “size” refer to the largest distance between one side to the other side of a given particle. In a round or close to round particle (e.g. spherical or oblong particle) the size and the diameter are mathematically equal, as described hereinbelow. In some embodiments, the term “particle size” refers to average cross section size of the nanoparticles. The term “dry diameter” is art-recognized and is used herein to refer to the physical diameter. The size of the particles may be evaluated using transmission electron microscopy (TEM) or scanning electron microscopy (SEM) imaging. In the absence of agglomerations, there are other techniques to define the particle sizes and distribution.

In some embodiments, at least 80%, at least 85%, at least 87%, at least 90%, at least 92%, at least 95%, at least 97%, at least 98%, or at least 99% of the particles of the invention have a particle size (e.g., an average size of the primary particles) of between 5 and 900 nm, between 5 and 900 nm, between 5 and 10 nm, between 10 and 50 nm, between 20 and 80 nm, between 20 and 150 nm, between 20 and 200 nm, between 10 and 200 nm, between 20 and 60 nm, between 20 and 40 nm, between 40 and 60 nm, between 50 and 100 nm, between 100 and 200 nm, between 200 and 300 nm, between 300 and 400 nm, between 400 and 500 nm, between 500 and 700 nm, or between 700 and 900 nm, including any range or value therebetween. In some embodiments, at least 80%, at least 85%, at least 87%, at least 90%, at least 92%, at least 95%, at least 97%, at least 98%, or at least 99% of the particles of the invention have a particle size (e.g., an average size of the primary particles) of between 5 and 200 nm, between 5 and 200 nm, between 5 and 10 nm, between 10 and 50 nm, between 20 and 80 nm, between 20 and 150 nm, between 50 and 100 nm, between 100 and 200 nm, including any range or value therebetween. Each possibility represents a separate embodiment of the invention.

In some embodiments, the term “average cross section size” may refer to either the average of at least e.g., 80%, 90%, or 95% of the particles, or in some embodiments, to the median size of the plurality of nanoparticles. In some embodiments, the term “average cross section size” refers to a number average of the plurality of nanoparticles. In some embodiments, the term “average cross section size” may refer to an average diameter of substantially spherical particles.

The term “particles” as used herein refers to a discrete submicron-particle or a nanoparticle of the invention, as well as to the aggregates or agglomerates thereof. In some embodiments, the term “particle” refers to a primary particle. In some embodiments, the primary particles are capable of aggregation, resulting in the formation of aggregates (or secondary particles). The particle of the invention may have any tri-dimensional geometrical shape, such as a sphere, an ellipsoid, oblong, or irregular shape, etc. In some embodiments, the particles of the invention have substantially the same geometrical shape or different shapes. In some embodiments, the particles of the invention are devoid of a uniform geometrical shape.

In some embodiments, the particles of the invention are in a solid state. In some embodiments, the composition comprising the particles of the invention is in a form of a powder (e.g., dry powder, having a water content as described herein).

In some embodiments, the divalent metal carbonate specie is or comprises calcium carbonate. In some embodiments, the divalent metal carbonate specie is manganese carbonate. In some embodiments, carbonate specie of the first divalent metal is zinc carbonate. In some embodiments the carbonate specie of the first divalent metal comprises a mixture of a first divalent metal and a second divalent metal, and optionally a third metal(s).

In some embodiments, the particle is substantially uniform. In some embodiments, the first divalent metal, the second divalent metal, and optionally the third metal are uniformly distributed within the particle of the invention. In some embodiments, the particle is substantially homogeneous throughout the entire particle volume, wherein the term “homogenous” refers to substantially uniform distribution of: (i) the stabilizer; and/or (ii) the metal (e.g., first divalent metal, second divalent metal and/or third metal(s)) within the entire volume of the particle and/or of the composition comprising thereof. In some embodiments, the stabilizer is located within the entire volume of the particle.

In some embodiments, the stabilizer and one or more divalent metal(s) are uniformly mixed within the entire particle. In some embodiments, the stabilizer is substantially uniformly distributed within a matrix formed by the amorphous metal(s) carbonate. In some embodiments, the stabilizer is chemically bound to the metal cation(s) via a covalent bond and/or via an ionic bond.

In some embodiments, the particle is substantially homogeneous throughout the entire particle volume and is devoid of a shell. In some embodiments, the particle is devoid of an external matrix. In some embodiments, the particle is substantially devoid of layers.

In some embodiments, the particle further comprises a shell, wherein the shell substantially comprises the stabilizer and/or the second amorphous metal carbonate (e.g. the carbonate of the second metal and/or of a carbonate of an additional metal). In some embodiments, the stabilizer and or the second metal is located at the outer portion of the particle (e.g. within the shell). In some embodiments, the stabilizer and/or the second metal is located within the entire volume of the particle and at the outer portion (e.g. shell) of the particle. Based on experimental evidence obtained by the inventors (see Example 6), it has been concluded that the stabilizer and/or the second metal is located in the interior and at the outer portion of the particle.

Furthermore, for an industrial scale synthesis it is of great importance to implement a sufficient amount of stabilizer (such as disclosed herein) during the manufacturing process, so as to facilitate a substantial incorporation of the stabilizer into the entire particle. As opposed, a particle having the stabilizer adsorbed to the outer portion (shell), is characterized by inferior stability, such as due to crystallization of the amorphous carbonate during the drying step.

In some embodiments, the stable particle of the invention substantially comprises the stabilizer located in the entire (or core) portion of the particle. In some embodiments, at least 50%, at least 60%, at least 70%, at least 80%, at least 90%, at least 95% by weight of the stabilizer is located in the entire portion (core) of the particle, including any range between.

In some embodiments, the particle is a core-shell particle, wherein the core is substantially homogenous and comprises the one or more amorphous divalent metal(s) carbonate, and the stabilizer, as described herein. In some embodiments, the shell substantially comprises the stabilizer, an amorphous metal carbonate, a metal oxide/metal hydroxide and optionally. In some embodiments, the core comprises the first divalent metal and the shell substantially comprises the second divalent metal and optionally the stabilizer.

In some embodiments, the ratios between the stabilizer, carbonate, and the divalent metal(s) within the core, are as described herein (in the “Particle” section). In some embodiments, the core-shell particle comprises an outer shell, wherein the shell substantially comprises the stabilizer and/or the second or more metal carbonates. It is presumed, that the core-shell particle is obtained as a result of two-step stabilizer and or second or more metal addition, as described in the method section. It is further postulated, that the outer shell provides a moisture barrier to the entire particle, thus inhibiting particle's dissolution and/or destabilization (e.g., by transformation of the amorphous metal carbonate into a crystalline state). Accordingly, it is presumed that the outer shell prolongs particle's stability upon prolonged storage and/or upon contact with an aqueous solution (e.g., in a suspension).

In some embodiments, a w/w ratio between the core and the shell is between 1000:1 and 10:1, between 1000:1 and 100:1, between 1000:1 and 500:1, between 500:1 and 100:1, between 100:1 and 50:1, between 50:1 and 10:1, between 50:1 and 30:1, between 30:1 and 10:1, including any range between.

In some embodiments, a w/w ratio between the stabilizer within the core and the stabilizer within the shell is between 20:1 and 1:1, between 20:1 and 10:1, between and 5:1, between 5:1 and 3:1, between 3:1 and 1:1, between 5:1 and 2:1, between 2:1 and 1:1, between 20:1 and 1:20 including any range between.

In some embodiments, at least 80%, at least 90%, at least 95%, at least 97%, at least 99% by weight of the shell is composed of the stabilizer and/or a salt thereof.

In some embodiments, the particle further comprises a metal salt. In some embodiments, the metal salt comprises a metal cation (e.g., divalent, mono-valent, and/or trivalent metal) and an anion (e.g., a counter anion). In some embodiments, the anion is selected from a halide (e.g., chloride, fluoride, bromide, iodide), acetate, sulphate, nitrate, citrate, or any combination thereof.

In some embodiments, the particle further comprises an anion. In some embodiments, the anion is selected from halide (e.g., chloride, fluoride, bromide, iodide), acetate, sulphate, nitrate, citrate, or any combination thereof.

In some embodiments, the particle further comprises: (i) an meta-oxygen-metal bonds (also referred to herein as, metal oxide) of the first divalent metal and/or of the second metal or one of the metals with the same type of metal, and optionally of the third metal; (ii) a hydroxyl (also referred to herein as, metal hydroxide) of the first divalent metal and/or of the second metal, and optionally of the third or more metals, or both (i) and (ii). In some embodiments, a w/w content of any of metal oxide and/or metal hydroxide within the particle and/or within the composition comprising thereof is between 1 to 30 mole % of the total metal quantity in the particle.

In some embodiments, at least 70%, at least 75%, at least 80%, at least 85%, at least 87%, at least 90%, at least 92%, at least 95%, at least 97%, at least 98%, at least 99% by weight of the divalent metal carbonate specie is amorphous, including any range or value therebetween. Each possibility represents a separate embodiment of the invention.

In some embodiments, at least 70%, at least 75%, at least 80%, at least 85%, at least 87%, or at least 90% by weight of the composition is amorphous, or any value and range therebetween. Each possibility represents a separate embodiment of the invention. In some embodiments, the crystallinity of the metal carbonate compositions, as disclosed herein, are determined by an X-Ray Powder Diffraction (XRD). In some embodiments, the term “amorphous” is referred to a non-crystalline composition (e.g., devoid of a crystal lattice). In some embodiments, the amorphous phase contains domains that are in the range of 1 to nm that have observed organized order like in a crystalline phase but there is no significant or completely no signature of crystalline X-ray or electron diffraction patterns.

In some embodiments, the composition of the invention comprising amorphous carbonate specie of the first divalent metal, is characterized by an XRD pattern being substantially devoid of a corresponding peak of a crystal lattice of the divalent metal carbonate. In some embodiments, the XRD pattern of the composition of the invention (e.g., powderous composition) is obtained as described hereinbelow (see Materials and Methods). In some embodiments the crystallized fraction of the composition represents XRD pattern with corresponding peaks at the right position that are wide, in their base, indicating the nanometric size of the crystalline grains.

By “substantially devoid of a corresponding peak” meant that an XRD pattern of the composition of the invention does not include at least one main corresponding peak of a crystalline phase that can be integrated by the XRD analyzer and is not integrated above 30% of an experimental curve established by mixing various ration of a fully amorphous phase and the corresponding crystalline phase of the material. In another embodiment, by “substantially devoid of” it is meant no more than 30, 20% or 10% of the crystalline divalent metal carbonate, by weight. In another embodiment, by “substantially devoid of” it is meant no more than 5% of the crystalline divalent metal carbonate, by weight. In another embodiment, by “substantially devoid of” it is meant no more than 1% of the crystalline divalent metal carbonate, by weight.

In some embodiments, the particle of the invention has a water content between 1 and 30%, between 1 and 5%, between 5 and 8%, between 8 and 10%, between 10 and 15%, between 15 and 18%, between 18 and 20%, between 20 and 30%, by weight of the particle including any range or value therebetween.

In some embodiments, the water content of the particle is at most 20%, at most 18%, at most 15%, at most 10%, at most 8%, by weight of the particle including any range or value therebetween.

As used herein, the term “water content” refers to any water molecules that are physically or chemically (e.g. forming a complex or an adduct of the water with metal atom, or bonded by hydrogen bonding) bound to the particle of the invention. In some embodiments, the water content comprises water molecules physically adsorbed to the external surface of the particle by electrostatic, hydrophilicity, or hydrogen bonding. Such water molecules can be vaporized in the temperature range between room temperature and 150 C. It can be water molecules that are trapped in nanometric pores within the particles or complexed (forming an adduct) to some of the metallic atoms of the carbonate molecular network or forming hydrogen bonding with hydroxyl or oxygen atoms. Such water is typically vaporized at 150 to 250 C. In some embodiments, the water content is derived from hydroxy groups bonded to either the metal, phosphorous or carbon atoms of the composition, which can be condensed and form M-O—C, M-O—P and M-O-M bonds while releasing water molecules, typically between 200 and 350 C.

In some embodiments, the particle of the invention comprises the amorphous metal carbonate, wherein the metal is bound to between 1 and 30 mol % of hydroxyl groups (also referred to herein as “water content”). In some embodiments, the hydroxyl groups are covalently bound to the metal, so as to form e.g., Mg—OH or Zn—OH, bonds. In some embodiments, the metal is bound to hydroxyl groups derived from organic hydroxy compounds, comprising functional groups derived from phosphorus (P—OH), sulfur (S—OH), carbon (C—OH), organic acid (C—OH), and/or carboxyl groups, or any combination thereof.

In some embodiments, the particle comprises chemical groups integrated in the particle's molecular network, consisting of hydroxyl, complexed water (“hydrated”, “adduct”, “ligand”) bonded to the metal atom), oxide or oxo groups and bonds. In some embodiments, the water content further comprises any water molecules adsorbed or embedded within the particle of the invention. Water and/or oxide content can be detected by thermal gravimetric analysis (TGA), including estimation of the exact content of each specie (such as adsorbed water, complexed water, hydroxylated metal content, etc.).

In some embodiments, the particles of the invention are substantially devoid of hollow particles. In some embodiments, the particles of the invention are substantially devoid of mesoporous particles. In some embodiments, the particles of the invention are substantially non-porous. In some embodiments, formed nanopores are in the form of closed porosity, i.e, they are not prone to adsorption or desorption of gases and liquids in ambient conditions. In some embodiments, the porosity of the particles is between 0.1 and 30 vol %, between 1 and 5 vol %, between 5 and 10 vol %, between and 20 vol %, including any range between.

In some embodiments, the particles and/or the composition comprising thereof is characterized by a BET surface area of at most 200 m²/g, at most 150 m²/g, at most 100 m²/g, at most 50 m²/g, including any range between.

In some embodiments, the particles and/or the composition comprising thereof is characterized by a BET surface area between 20 and 200 m²/g, between 30 and 150 m²/g, between 20 and 150 m²/g, between 20 and 110 m²/g, between 30 and 150 m 2/g, between 30 and 100 m²/g, between 100 and 110 m²/g, between 110 and 150 m²/g, between 150 and 200 m²/g, including any range between.

In some embodiments, the particles and/or the composition comprising thereof is characterized by an average pore size of between 0.5 and 10 nm, of between 1 and 5 nm, of between 2 and 10 nm, of between 2 and 3 nm, of between 1 and 3 nm, including any range between.

In some embodiments, the particles of the invention comprise a synthetic amorphous divalent metal carbonate. In some embodiments, the amorphous divalent metal carbonate disclosed herein is a synthetic divalent metal carbonate. In some embodiments, the amorphous divalent metal carbonate disclosed herein is manufactured according to any one of the methods disclosed herein. In some embodiments, the particles of the invention are substantially devoid of natural amorphous divalent metal carbonate.

In some embodiments, the particle of the invention (in a form of a powder is stable for at least 50 d, at least 100 d, at least 200 d, at least 300 d, at least 1 year (y), at least 2 y, at least 3 y, including any range or value therebetween.

In some embodiments, the particle of the invention (e.g., comprising at least or at least 1 mol % of the stabilizer relative to the metal) in a form of an aqueous formulation (such as a suspension) is stable for at least 24 h, at least 2 days (d), at least 7 d, at least 10 d, at least 20 d, at least 30 d, at least 40 d, at least 50 d, including any range or value therebetween.

In some embodiments, the particle of the invention refers to as “stable” in an aqueous formulation indicates that at least about 80% by total weight of the divalent metal carbonate remains amorphous upon storage of the aqueous formulation at a temperature less than 40° C., or less than 35° C., less than 30° C. or at an ambient temperature (between about 20 and about 25° C.), for a time period of at least 24 h, at least 2 days (d), at least 7 d, including any range or value therebetween.

The term “stable” (or the term stable particle), including any grammatical form thereof, as used herein indicates that the calcium carbonate is maintained in the amorphous form for a long period of time (under appropriate conditions, as described herein) in the solid form having less than or about 30%, about 20%, about 15%, about 10%, about 5% of crystalline divalent metal carbonate by total weight of the particle and/or of the composition comprising thereof. Specifically, the term “stable” (or the term stable particle), including any grammatical form thereof, as used herein indicates that at least 80%, or at least 95% by total weight of the divalent metal carbonate remains amorphous upon storage of the composition under appropriate conditions for at least 1 month, at least 100 d, at least 200 d, at least 300 d, at least 1 year (y), at least 2 y, at least 3 y, including any range or value therebetween.

In some embodiments, a stable particle is substantially devoid of disintegration, agglomeration, and/or crystallization of the divalent metal carbonate specie. In some embodiments, a stable particle substantially retains its physical and/or chemical properties. In some embodiments, a stable particle substantially retains its structure and/or geometrical shape. In some embodiments, a stable particle substantially retains its size or any other physical parameter. In some embodiments, a stable particle refers to chemical stability of the particle (e.g., being devoid of oxidation). In some embodiments, a stable particle is a chemically inert particle.

In some embodiments, the particle is stable upon storage under appropriate conditions, for a time period as described herein. In some embodiments, appropriate conditions comprise a temperature of at most 40° C. at most 30° C. at most 10° C. at most ° C., at most −10° C. including any range or value therebetween. In some embodiments, appropriate conditions comprise a temperature of between −80° C. and 50° C., including any range or value therebetween.

In some embodiments, the appropriate conditions comprise storage conditions. In some embodiments, the appropriate conditions comprise on the shelf conditions. In some embodiments, the appropriate conditions comprise the conditions wherein the end user stores or preserves the herein disclosed composition.

In some embodiments, appropriate conditions comprise conditions wherein at the activity of the herein disclosed composition is preserved or maintained. In some embodiments, appropriate conditions comprise conditions wherein the particle is kept in close and dry conditions (e.g., humidity content below 30%, below 20%, or below 10%, etc.), maintained using vacuum, a separated container of a water scavenger, e.g., silica gel, molecular sieves, or aerosol.

In some embodiments, appropriate conditions comprise a suspension with pH of at least 7.5, at least 8, at least 9, at least 10, including any range or value therebetween.

In some embodiments, the particle (e.g., the primary particle and/or the secondary particle) undergoes a slight dissolution at a pH below 9, 8, 7 or 6 until the above-mentioned pH levels are reached.

In some embodiments, appropriate conditions comprise a relative humidity ranging from 10 to 60% including any range or value therebetween.

In some embodiments, at least 10%, at least 20%, at least 30%, at least 50%, at least 60%, at least 70%, at least 80%, at least 90% of the particles are stable, wherein stable is as described hereinabove.

In some embodiments, the stable particle of the invention is characterized by a water content of less than 20%, less than 18%, less than 15%, less than 13%, less than 10% by weight of the particle, including any range or value therebetween, wherein stable is as described herein.

### Amorphous Single Divalent Metal Carbonate Particle

In one aspect of the invention, there is provided a plurality of particles of the invention (also referred to herein as the single divalent metal particles), wherein each particle comprises a divalent metal carbonate specie (e.g., with or without any additional types of bonding), and the stabilizer of the invention; wherein at least 70% by weight of the divalent metal carbonate (also referred to herein as a “carbonate specie”) is in an amorphous state, and the divalent metal is selected from the group consisting of Zn, Fe, Cu, Co, Ni, Mn, and Cr; and a molar ratio between the divalent metal and the stabilizer is between 100:1 and 4:1. In some embodiments, the single divalent metal particles comprise a single divalent metal specie selected from the group consisting of Zn, Cu, Co, Ni, Mn, and Cr. In some embodiments, the single divalent metal particles comprise the single divalent metal specie as described herein, and at least 5%, at least 8%, at least 10% by weight of the stabilizer, wherein the stabilizer is as described herein. In some embodiments, the divalent metal of the invention is or comprises a metal in an oxidation state of +2.

In another aspect of the invention, there is provided a plurality of single divalent metal particles of the invention, wherein each particle comprises a divalent metal carbonate specie (e.g., with or without any additional types of bonding), and the stabilizer of the invention; wherein at least 70% by weight of the divalent metal carbonate is in an amorphous state, and the divalent metal is selected from the group consisting of Mg, Zn, Cu, Co, Ni, Mn, and Cr; and a molar ratio between the divalent metal and the stabilizer is between 100:1 and 4:1; and wherein the plurality of particles is characterized by BET surface area of at most 500 m²/g, at most 200 m²/g, at most 100 m²/g, or at least 25 m²/g, at least 50 m²/g, at least 100 m²/g including any range between. In some embodiments, the single divalent metal particles comprise a single divalent metal specie selected from the group consisting of Mg, Zn, Cu, Co, Ni, Mn, and Cr, and are characterized by BET surface area of at most 500 m²/g, at most 200 m 2/g, at most 100 m²/g, or at least 25 m²/g, at least 50 m²/g, at least 100 m²/g, including any range between.

In some embodiments, the molar ratio between the divalent metal and the stabilizer within the single divalent metal particle is between 100:1 and 100:25, between 100:1 and 100:5, between 100:5 and 100:7, between 100:5 and 100:25, between 100:3 and 100:25, between 100:7 and 100:15, between 100:7 and 100:10, between 100:10 and 100:20, between 100:10 and 100:15, between 100:15 and 100:25, between 100:20 and 100:25, including any range between.

In some embodiments, the molar ratio between the stabilizer and the divalent metal is at least 1%, at least 3%, at least 4%, at least 5%, at least 7%, at least 10%, including any range between.

In some embodiments, the weight ratio between the total divalent metal content (e.g. comprising the first divalent metal, and one or more of the second divalent metal(s)), and the stabilizer is between 5 and 20%, between 5 and 8%, between 8 and 10%, between 8 and 20%, between 10 and 20%, between 20 and 30%, including any range between.

In some embodiments, the divalent metal carbonate comprises a carbonate specie and a divalent metal atom. In some embodiments, the divalent metal atom is a single divalent metal.

In some embodiments, the particle size is as described hereinabove.

In some embodiments, the particle comprises a stabilizer, wherein the stabilizer is as described herein. In some embodiments, the molar ratio between the first divalent metal and the stabilizer within the particle is as described hereinabove. In some embodiments, the molar ratio between the divalent metal and the stabilizer is as described hereinabove.

In some embodiments, the divalent metal is Zn and the molar ratio between the divalent metal and the stabilizer (e.g., triphosphate) is as described herein.

In some embodiments, a w/w ratio between the stabilizer and the divalent metal within the particle is as described herein.

In some embodiments, a water content of the particle is as described herein (e.g., between 1 and 30% by weight).

In some embodiments, a w/w concentration of the divalent metal within the particle is between 20 and 70%, between 20 and 30%, between 30 and 35%, between and 38%, between 38 and 40%, between 40 and 50%, between 50 and 60%, between and 70%, including any range or value therebetween, by total dry weight of the particle.

In some embodiments, a w/w concentration of carbonate within the single divalent metal particle is between 1 and 40%, between 1 and 10%, between 10 and 40%, between 10 and 20%, between 20 and 40%, between 20 and 30%, between 30 and 40%, including any range or value therebetween, by total dry weight of the particle.

In some embodiments, a molar ratio between carbonate and the divalent metal within the single divalent metal particle is between 10:1 and 1:1, between 10:1 and 8:1, between 10:1 and 5:1, between 8:1 and 5:1, between 5:1 and 3:1, between 3:1 and 1:1, including any range or value therebetween.

In some embodiments, a w/w ratio of the divalent metal relative to the total metal content of the particle is between 50 and 99%, between 50 and 60%, between 60 and 70%, between 70 and 80%, between 80 and 85%, between 85 and 90%, between and 92%, between 92 and 95%, between 95 and 97%, between 97 and 99%, including any range or value therebetween, by total dry weight of the particle.

In some embodiments, the particle is substantially devoid of a divalent metal oxide. In some embodiments, the content of the divalent metal oxide within the particle is up to 10%, up to 8%, up to 5%, up to 1%, up to 0.1%, including any range or value therebetween, relative to the weight content of the divalent metal.

In some embodiments, the single divalent metal particles are substantially stable for at least 1 month, at least 3 m, at least 6 m, at least 12 m in a dry state under appropriate conditions, as described herein.

In some embodiments, the single divalent metal particles are substantially stable for at least 1 day, at least 2 d, at least 3 d, at least 1 week, at least one month in an aqueous suspension under appropriate conditions, as described herein.

In some embodiments, the content (e.g., mol ratio, or w/w ratio) of the stabilizer within the single divalent metal particle sufficient for stabilizing the particle in the dry state and/or suspension is as described herein.

According to some embodiments, there is provided a method of manufacturing the single divalent metal particles, the method comprising contacting a first aqueous composition comprising divalent metal cations (e.g. in a form of a dissociated divalent metal salt, including a counter anion which is not carbonate, such as halide (e.g., chloride, fluoride, bromide, iodide), acetate, sulphate, nitrate, citrate, or any combination thereof) with a second aqueous composition comprising carbonate anions under suitable conditions, thereby obtaining the single divalent metal particles. In some embodiments, contacting results in the formation of a mixture. In some embodiments, the first aqueous composition and/or second aqueous composition further comprises the stabilizer dissolved or dispersed therewithin. In some embodiments, the method further comprising adding a stabilizer solution to the mixture subsequently or simultaneously with the contacting step.

In some embodiments, the method further comprising separating the particles from the aqueous solution, to obtain the single divalent metal particles. In some embodiments, the method further comprising drying the particles under conditions sufficient for obtaining dry single divalent metal particles of the invention, characterized by a water content as disclosed herein (up to 30% by weight).

In some embodiments, contacting is performed by mixing, stirring, shaking, or any other method known in the art. In some embodiments, suitable conditions comprise a temperature between 3 and 50° C., between 3 and 10° C., between 3 and 30° C., between 3 and 25° C., between 10 and 50° C., between 10 and 30° C., between 10 and 40° C., between 3 and 10° C., between 10 and 20° C., between 20 and 50° C., between 20 and 30° C., including any range between. In some embodiments, suitable conditions comprise a contacting time of at least 1 second, at least 1 minute, or more. In some embodiments, contacting time is sufficient for forming the particles of the invention. In some embodiments, contacting time is sufficient for reacting at least 50%, at least 60%, at least 80%, at least 90%, at least 95%, at least 99% including any range or value therebetween, or more of the initial divalent metal content within the first aqueous composition with the carbonate. In some embodiments, contacting time is sufficient for converting at least 50%, at least 60%, at least 80%, at least 90%, at least 95%, at least 99% including any range or value therebetween, or more of the initial divalent metal into the amorphous divalent metal carbonate (e.g. in the form of particles of the invention). Conversion can be monitored by any analytical technique known in the art (e.g. ICP, NMR, etc.). Furthermore, conversion can be monitored by monitoring the precipitation of the particles form the mixture. Thus, the end point of the particle formation step is indicated by a substantial arrest of the precipitation.

In some embodiments, the first aqueous composition and the second aqueous composition are aqueous solutions. In some embodiments, the first aqueous composition and the second aqueous composition comprise a sufficient amount of each the divalent metal and/or carbonate. In some embodiments, a concentration of the divalent metal cations within the first aqueous composition is between 1 mM and 10M, is between 1 mM and 10 mM, is between 10 mM and 100 mM, is between 100 mM and 500 mM, is between 100 mM and 300 mM, is between 300 mM and 500 mM, is between 500 mM and 1M, including any range or value therebetween. In some embodiments, a concentration of carbonate within the second aqueous composition is between 1 mM and 10M, is between 1 mM and 10 mM, is between 10 mM and 100 mM, is between 100 mM and 500 mM, is between 100 mM and 300 mM, is between 300 mM and 500 mM, is between 500 mM and 1M, including any range or value therebetween.

In some embodiments, a molar ratio of the stabilizer relative to the divalent metal within the mixture is between 0.5 and 30%, between 0.5 and 10%, between 0.5 and 1%, between 1 and 30%, between 1 and 5%, between 5 and 10%, between 10 and 30%, at least 0.5%, at least 0.8%, at least 1%, at least 1.1%, at least 1.5%, at least 2%, at least 4%, at least 8%, including any range or value therebetween.

In some embodiments, a molar ratio of the carbonate relative to the divalent metal within the mixture is between about 0.5:1 and about 2:1, between about 0.5:1 and about 0.7:1, between about 0.7:1 and about 1:1, between about 1:1 and about 1:1.5, between about 1:1.5 and about 1:2, including any range or value therebetween.

### Mixed Amorphous Metal Carbonate Particles

In another aspect of the invention, the particle of the invention comprises a mixture of a first divalent metal carbonate and a second divalent metal carbonate, wherein the first divalent metal and the second divalent metal are each independently selected from: Ca, Zn, Fe, Cu, Co, Ni, Mn, or Cr, wherein at least 70%, at least 80%, at least 90%, at least 95% by weight of carbonate (or metal carbonate) within the particle and/or the composition is amorphous. In another aspect of the invention, the particle of the invention comprises a mixture of a first divalent metal, a second divalent metal and a carbonate specie, wherein the first divalent metal and the second divalent metal are each independently selected from: Ca, Zn, Fe, Cu, Co, Ni, Mn, or Cr, wherein at least 70%, at least 80%, at least 90%, at least 95% by weight of the carbonate specie within the particle is amorphous, wherein the first divalent metal and the second divalent metal are chemically distinct metal species.

In some embodiments, the particle of the invention comprising a mixture of a first divalent metal carbonate and a second divalent metal carbonate, is referred to herein as a mixed particle. In some embodiments, the mixed particle is an amorphous metal carbonate particle. In some embodiments, the mixed particle is an amorphous metal carbonate nanoparticle. In some embodiments, the first divalent metal is selected from the group comprising Zn(II), Fe(II), Cu(II), Co(II), Ni(II), Mn(II), and Cr(II). In some embodiments, the second divalent metal is selected from the group comprising Zn(II), Fe(II), Cu(II), Co(II), Ni(II), Mn(II), and Cr(II), or any combination thereof. In some embodiments, the second divalent metal and the first divalent metal of the invention are metals in an oxidation state of +2.

In some embodiments, the content (e.g. mol ratio, or w/w ratio) of the second divalent metal within the mixed particle sufficient for stabilizing the particle in the dry state and/or suspension is as described herein.

In some embodiments, the mixed particle of the invention is devoid of the stabilizer, and wherein a molar ratio between the first divalent metal and the second divalent metal within the particle is between 10:0.3 and 10:2, between 10:0.3 and between 10:0.4 and 10:0.5, between 10:0.5 and 10:0.7, between 10:0.7 and between 10:0.8 and 10:1, between 10:1 and 10:2, including any range or value therebetween.

In some embodiments, a mol ratio between the second divalent metal and the first divalent metal within the mixed particle is at least 0.5%, at least 1%, at least 5%, at least 10%, at least 15%, or more, including any range or value therebetween. In some embodiments, a weight ratio between the second divalent metal and the first divalent metal within the mixed particle is between 0.5 and 40%, between 1 and 30%, between and 20%, between 10 and 40%, between 0.5 and 30%, between 10 and 20%, between and 25%, between 5 and 20%, between 20 and 40%, between 20 and 30%, between and 40%, including any range or value therebetween.

In some embodiments, a weight ratio between the second divalent metal and the first divalent metal within the mixed particle is at least 6%, at least 8%, at least 10%, or more, including any range or value therebetween. In some embodiments, a weight ratio between the second divalent metal and the first divalent metal within the mixed particle is between 6 and 40%, between 6 and 30%, between 6 and 10%, between 8 and 40%, between 8 and 40%, between 8 and 10%, between 10 and 30%, between 10 and 20%, between 10 and 25%, between 8 and 20%, between 20 and 40%, between 20 and 30%, between 30 and 40%, including any range or value therebetween.

In some embodiments, the mixed particle of the invention is as described herein, wherein a weight ratio of the second divalent metal relative to the first divalent metal within the particle is at least 6%, at least 8%, at least 9%, at least 10%, including any range or value therebetween. Exemplary mixed particles with 10-20% weight content of the second divalent metal (relative to the first divalent metal) have been successfully synthesized and their stability in the dry solid state has been confirmed. The inventors presume that a weight ratio of the second metal below 6% would result in a particle with an inferior stability and/or would be irrelevant for industrial scale application.

In some embodiments, the first divalent metal is Ca, and the second divalent metal is selected from: Zn, Fe, Cu, Co, Ni, Mn, or Cr. In some embodiments, the first divalent metal is devoid of Ca, and the second divalent metal is selected from: Mg, Zn, Fe, Cu, Co, Ni, Mn, or Cr.

According to another embodiment, the particle of the invention (also referred to herein as “mixed stabilized particle”) comprises a carbonate specie comprising a first metal and a second metal, and further comprises a stabilizer; wherein at least 70%, or at least 99% by weight (including any range between) of the carbonate specie within the composition is amorphous; and wherein any one of the first divalent metal and the second divalent metal is selected from: Ca, Zn, Fe, Cu, Co, Ni, Mn, Mg, and Cr, or any combination thereof. In some embodiments, there is provided a composition (e.g. a liquid or a powderous composition) comprising a plurality of mixed stabilized particles. In some embodiments, the first metal is selected from: Ca, Zn, Fe, Cu, Co, Ni, Mn, and Cr, and the second metal is Mg, and a molar ratio between the stabilizer and the first divalent metal within the mixed stabilized particles is between 1:100 and 25:100, including any range between.

In some embodiments, a weight ratio between the second divalent metal and the first divalent metal within the particle (e.g., the stabilized mixed particle) is at least at least 1%, at least 1.5%, at least 2%, at least 3%, at least 5%, including any range or value therebetween.

In some embodiments, a weight ratio between the divalent metal and the first divalent metal within the stabilized mixed particle is between 1 and 40%, between 1 and 30%, between 1 and 10%, between 10 and 30%, between 1 and 20%, between 2 and 10%, between 2 and 30%, between 2 and 20%, between 10 and 25%, between 8 and 20%, between 20 and 40%, between 20 and 30%, between 30 and 40%, including any range or value therebetween.

In some embodiments, the stabilized mixed particle further comprises a stabilizer, wherein the stabilizer is as described herein. In some embodiments, the molar ratio between the first divalent metal and the stabilizer within the stabilized mixed particle is as described hereinabove. It is postulated, that the stabilizer substantially enhances particle's stability in an aqueous formulation (e.g., suspension or dispersion) and/or highly moist environment. It is postulated, that the stabilizer substantially enhances particle's stability during large scale manufacturing, especially during the drying step of the scaled product.

In some embodiments, a w/w ratio between the stabilizer and the first divalent metal within the mixed stabilized particle is at least 4%, at least 5%, at least 6%, at least 7%, at least 8%, at least 9%, at least 10%, including any range between.

In some embodiments, a molar ratio between the stabilizer and the total metal content (the first metal and the second metal) within the mixed stabilized particles is at least 5%, at least 6%, at least 7%, at least 10%, including any range between.

In some embodiments, a weight ratio between the stabilizer and the total metal content (the first metal and the second metal) within the mixed stabilized particles is at least 8%, at least 10%, at least 15%, at least 10%, including any range between.

In some embodiments, the first divalent metal is Ca and the molar ratio between Ca and the stabilizer (e.g., triphosphate) is between 100:1 and 100:30, between 100:1 and 100:20, between 100:0.5 and 100:10, between 100:1 and 100:10, between 100:2 and 100:10, including any range or value therebetween.

In some embodiments, a w/w ratio between the stabilizer and the first divalent metal within the mixed stabilized particle is between 0.1:10 and 2.5:10, between 0.1:10 and 0.7:10, between 0.7:10 and 0.9:10, between 0.9:10 and 1.1:10, between 1.1:10 and 1.5:10, between 1.5:10 and 2:10, between 2:10 and 2.5:10, including any range or value therebetween.

In some embodiments, a w/w ratio between the stabilizer and the first divalent metal within the particle (e.g., the mixed stabilized particle) is at least 5%, at least 7%, at least 8%, at least 9%, at least 10%, at least 20%, at least 25%, including any range or value therebetween.

In some embodiments, a molar ratio between the stabilizer and the first divalent metal within the particle (e.g., the mixed stabilized particle) is at least 0.5%, at least 0.7%, at least 0.8%, at least 0.9%, at least 1%, including any range or value therebetween.

In some embodiments, the particle of the invention (e.g., the mixed stabilized particle) comprises a mixture of a first divalent amorphous metal carbonate and a second or more amorphous metal carbonates, wherein the first divalent metal is Ca, and the second divalent metal is Mg; and wherein the particle further comprises a stabilizer of the invention; wherein the particle is characterized by a BET surface area as described hereinabove; and wherein the particle is an amorphous metal carbonate particle. In some embodiments, the first divalent metal is Ca, and the second divalent metal is Mg, wherein a molar ratio between Mg and Ca is at least 1 mol %, and wherein the particle is characterized by a BET surface area as described hereinabove.

In some embodiments, the first divalent metal is Ca, and the second divalent metal is Mg, wherein the particle (e.g., the mixed stabilized particle or the mixed particle) is characterized by a BET surface area as described hereinabove, and wherein a molar ratio between Ca and Mg is between 5:1 and 2:1, between 5:1 and 3:1, between 5:1 and 4:1, between 4:1 and 2:1, between 4:1 and 3:1, between 3:1 and 2:1, including any range between.

In some embodiments, the first divalent metal is Ca, and the second divalent metal is Mg, wherein the particles (e.g., the mixed stabilized particles or the mixed particles) are characterized by a BET surface area as described hereinabove, wherein a molar ratio between Ca and Mg is between 5:1 and 2:1; wherein the particle is stable for at least 1 m, at least 1 y, including any range between upon storage in a powderous form under appropriate conditions (e.g., a temperature between −15 and 40° C., or between 0 and 25° C., or between 5 and 40° C., or between 0 and 30° C., including any range between).

In some embodiments, the first divalent metal is Ca, and the second divalent metal is selected from: Zn, Fe, Cu, Co, Ni, Mn, or Cr. In some embodiments, the first divalent metal is devoid of Ca, and the second divalent metal is selected from: Mg, Zn, Fe, Cu, Co, Ni, Mn, or Cr.

In some embodiments, the first divalent metal and the second divalent metal are as describe hereinabove. In some embodiments, the first divalent metal and the second divalent metal are independently selected from: Ca, Zn, Fe, Cu, Co, Ni, Mn, or Cr; wherein the first divalent metal and the second divalent metal are different metals. In some embodiments, the first divalent metal and the second divalent metal are independently selected from: Mg, Zn, Fe, Cu, Co, Ni, Mn, or Cr; wherein the first divalent metal and the second divalent metal are different metals.

In some embodiments, the first divalent metal is Ca. In some embodiments, the first divalent metal is Ca, and the second divalent metal is Zn and/or Fe. In some embodiments, the first divalent metal is Ca, and the second divalent metal is selected form Zn, Fe, Cr and Mg. In some embodiments, the first divalent metal is Mg, and the second divalent metal is Zn and/or Fe. In some embodiments, the first divalent metal is Zn, and the second divalent metal is Ca, Mg and/or Fe.

In some embodiments, the first divalent metal is or comprises a single divalent metal. In some embodiments, the particle (e.g., the mixed particle) further comprises a third metal. In some embodiments, the third metal comprises any of Ca, Zn, Fe, Cu, Co, Ni, Mn, Mg and Cr, or a combination thereof.

In some embodiments, the divalent metal carbonate specie of the first divalent metal and of the second metal are bound within the particle, wherein bound is via a covalent and/or ionic bonds. In some embodiments, the first divalent metal and the second metal are uniformly distributed within the particle. In some embodiments, the particle is in a form of a composite consisting of regions rich with one or more divalent metals or one or more stabilizers. In some embodiments, the particle is in a form of a composite, wherein the composite structure is in the form of a core-shell structure. In some embodiments, the first divalent metal and the second divalent metal, and the carbonate specie are bound together so as to form a composite. In some embodiments, the composite is in a form of a plurality of particles of the invention.

In some embodiments the presence of the second metal affects the overall aqueous solubility (or dispersibility) of the particle and/or composition comprising thereof, compared to each individually prepared metal carbonate. In some embodiments, the second metal (e.g. second divalent metal) is present within the particle in an amount sufficient for stabilizing the particle, wherein the sufficient amount is optionally predetermined by the chemical composition of the second metal. In some embodiments, the sufficient amount may vary, depending on the specific metal atom and/or an oxidation state thereof. In some embodiments, the sufficient amount is as described herein (e.g. at least 0.5 mol %, or at least 1 mol % relative to the first metal content within the particle or composition).

In some embodiments, the composition of the invention comprises a plurality of mixed particles of the invention comprising a carbonate specie of the first divalent metal and carbonate specie of the second divalent metal wherein at least a portion of the composition is amorphous. In some embodiments, amorphous is as described herein. In some embodiments, the composition and/or particle of the invention comprises a mixed divalent metal carbonate specie comprising the first divalent metal and the second or more divalent metal, wherein at least a portion of the mixed divalent metal carbonate specie is amorphous.

In some embodiments, the carbonate specie of the first divalent metal forms a matrix (or molecular network) within the particle of the invention. In some embodiments, the second divalent metal (or the carbonate specie of the second divalent metal) is enclosed by or embedded within the matrix of the particle. In some embodiments, the second divalent metal is incorporated within the matrix. In some embodiments, the second divalent metal is uniformly distributed within the matrix. In some embodiments, the matrix is dopped with the second divalent metal atom. In some embodiments, the particle with the first divalent metal is coated with the second divalent metal atom to form a core-shell particle.

In some embodiments, the composition of the invention comprises a chemically identical particles, or chemically distinct particles.

In some embodiments, a molar ratio between the first divalent metal and the second divalent metal within the particle (e.g., the mixed particle and/or stabilized mixed particle) is between 10:0.1 and 10:3, between 10:0.1 and 10:0.2, between 10:0.2 and 10:0.5, between 10:0.5 and 10:0.7, between 10:0.7 and 10:0.9, between 10:0.9 and between 10:1 and 10:1.5, between 10:1.5 and 10:2, between 10:2 and 10:3, between 10:0.7 and 10:5, between 10:0.7 and 10:4, between 10:0.7 and 10:3, including any range or value therebetween. In some embodiments, a molar ratio between the second divalent metal and the first divalent metal within the particle (e.g., the mixed particle and/or stabilized mixed particle) is at least 6%, at least 7%, at least 8%, at least 10%, including any range or value therebetween.

In some embodiments, a molar ratio between the second divalent metal and the first divalent metal within the particle (e.g., the mixed particle and/or stabilized mixed particle) is between 8 and 33%, between 6 and 50%, between 6 and 40%, between 8 and 40%, between 8 and 35%, between 8 and 33%, between 8 and 30%, between 10 and 40%, between 8 and 10%, between 10 and 20%, between 10 and 30%, including any range or value therebetween.

In some embodiments, a stable mixed particle and/or stabilized mixed particle comprises a first divalent metal carbonate and a second divalent metal carbonate, wherein a molar ratio between the second divalent metal and the first divalent metal within the particle is between 6 and 50%, between 8 and 50%, between 8 and 40%, between 8 and 35%, including any range or value therebetween, wherein “stable” refers to the stability of the powderous composition, as disclosed herein.

In some embodiments, a w/w ratio between the first divalent metal and the second divalent metal within the particle is between 10:0.1 and 10:2, between 10:0.1 and 10:0.2, between 10:0.2 and 10:0.5, between 10:0.5 and 10:0.7, between 10:0.7 and between 10:0.9 and 10:1, between 10:1 and 10:1.5, between 10:1.5 and 10:2, including any range or value therebetween. In some embodiments, a w/w ratio between the first divalent metal and the second divalent metal within the particle is between and 10:1.5, including any range or value therebetween.

In some embodiments, the particle size of the mixed stabilized particles or mixed particles is as described hereinabove (in the “Particle” section).

In some embodiments, the plurality of particles (e.g. mixed stabilized particles or mixed particles) is characterized by BET surface area of at most 500 m²/g, or at most 200 m²/g, or at most 100 m²/g.

In some embodiments, the particle of the invention (e.g., the mixed particle) comprises a mixture of a first divalent amorphous metal carbonate and a second amorphous divalent metal carbonate, wherein the first divalent metal is Ca, and the second divalent metal is Mg; wherein the particle is characterized by a BET surface area as described hereinabove; and wherein the particle is an amorphous metal carbonate particle. In some embodiments, the first divalent metal is Ca, and the second divalent metal is Mg, wherein a molar ratio between Mg and Ca is at least 1 mol %, and wherein the particle is characterized by a BET surface area as described hereinabove.

In some embodiments, a w/w concentration of carbonate within the mixed particle (e.g. mixed particle or stabilized mixed particle) is between 1 and 40%, between 1 and 10%, between 10 and 40%, between 10 and 20%, between 20 and 40%, between and 30%, between 30 and 40%, including any range or value therebetween, by total dry weight of the particle.

In some embodiments, a molar ratio between carbonate and the first divalent metal within the mixed particle (e.g., mixed particle or stabilized mixed particle) is between 10:1 and 1:1, between 10:1 and 8:1, between 10:1 and 5:1, between 8:1 and between 5:1 and 3:1, between 3:1 and 1:1, including any range or value therebetween.

In some embodiments, any one of the particles of the invention (e.g., mixed particle, and/or a mixed stabilized particle) is stable in form of powderous composition, wherein stable is as described herein. In some embodiments, any one of the particles of the invention (e.g. mixed particles and/or a mixed stabilized particle) is stable in form of an aqueous formulation for a time period ranging between 1 minute (min) and 24 h, between 1 min and 1 h, between 1 min and 10 min, between 1 min and 30 min, between 1 min and 48 h, between 1 min and 2 h, between 1 min and 10 h, between 1 min and between 1 and 20 h, between 10 and 24 h, between 10 and 48 h, including any range or value therebetween.

In some embodiments, the mixed particle and/or the mixed stabilized particle further comprises a third metal. In some embodiments, a molar ratio between the first divalent metal and the third metal within the particle is between 10:0.1 and 10:1, between 10:0.3 and 10:0.4, between 10:0.4 and 10:0.5, between 10:0.5 and 10:0.7, between 10:0.7 and 10:0.8, between 10:0.8 and 10:1, including any range or value therebetween.

In some embodiments, there is provided a stable dispersion of stabilized mixed particles comprises (i) a first divalent metal carbonate and a second divalent metal carbonate and optionally a third or more metals, wherein a molar ratio between the second divalent metal and the first divalent metal within the particle is between 1 and 50%, between 5 and 50%, between 5 and 40%, between 5 and 35%, including any range or value therebetween; and further comprising the stabilizer at a molar ratio between the stabilizer and the first metal of at least 0.5%, at least 0.7%, at least 0.8%, at least 0.9%, at least 1%, at least 5% including any range or value therebetween. In some embodiments, a dispersion stability of mixed particle refers to the stability of the aqueous dispersion, as disclosed herein (e.g. retention of at least about 80% of the amorphous metal carbonate for a time period of at least 7 days).

In some embodiments, a water content of the dry particles is as described herein (e.g., between 1 and 30% by weight).

In some embodiments, a w/w concentration of the first divalent metal within the particle is between 20 and 70%, between 20 and 30%, between 30 and 35%, between and 38%, between 38 and 40%, between 40 and 50%, between 50 and 60%, between and 70%, including any range or value therebetween, by total dry weight of the particle.

In some embodiments, a w/w ratio of the first divalent metal relative to the total metal content of the particle is between 50 and 99%, between 50 and 60%, between and 70%, between 70 and 80%, between 80 and 85%, between 85 and 90%, between and 92%, between 92 and 95%, between 95 and 97%, between 97 and 99%, including any range or value therebetween, by total dry weight of the particle.

In some embodiments, a w/w concentration of the second divalent metal within the particle is between 0.1 and 40%, between 0.1 and 30%, between 0.5 and 20%, between 1 and 10%, between 3 and 5%, between 5 and 10%, between 10 and 40%, including any range or value therebetween, by total dry weight of the particle.

In some embodiments, the particle is substantially devoid of a metal oxide or metal-oxo bonding. In some embodiments, the content of the metal oxide bonding within the particle is up to 10%, up to 8%, up to 5%, up to 1%, up to 0.1%, including any range or value therebetween, relative to the weight content of the divalent metal.

In some embodiments, the dry particles (mixed particles and mixed stabilized particles) are stable for at least 1 month, at least 6 m, at least 12 m, in a dry state when stored under appropriate conditions as described hereinabove.

In some embodiments, the composition of the invention is in a form of a kit comprising a first compartment comprising a divalent metal carbonate precursor (e.g. a divalent metal salt comprising a counterion which is not carbonate); and a second compartment comprising a mono-valent carbonate salt (e.g. sodium carbonate, or any alkali metal carbonate). In some embodiments, a w/w ratio between the divalent metal of the divalent metal carbonate precursor and the mono-valent carbonate salt within the kit is between about 0.5:1 and about 2:1, between about 0.5:1 and about 0.7:1, between about 0.7:1 and about 1:1, between about 1:1 and about 1:1.5, between about 1:1.5 and about 1:2, including any range or value therebetween.

In some embodiments, the first compartment and/or the second compartment further comprises the stabilizer. In some embodiments, a molar ratio between the divalent metal of the divalent metal carbonate precursor and the stabilizer within the kit is between 0.5 and 30%, between 0.5 and 10%, between 0.5 and 1%, between 1 and 30%, between 1 and 5%, between 5 and 10%, between 10 and 30%, at least 0.5%, at least 0.8%, at least 1%, at least 1.1%, at least 1.5%, at least 2%, at least 4%, at least 8%, including any range or value therebetween.

In some embodiments, the divalent metal carbonate precursor comprises a single divalent metal specie. In some embodiments, the divalent metal carbonate precursor comprises a plurality of divalent metal species (e.g. the first divalent metal and the second divalent metal as disclosed herein).

In some embodiments, a molar ratio between the first and the second divalent metal within the kit is as described herein (e.g. between 10:0.05 and 1:1 including any range between).

According to some embodiments, there is provided a method of manufacturing the mixed particles of the invention, the method comprising contacting a first aqueous composition comprising cations of the first divalent metal and of the second divalent metal (e.g. in a form of a dissociated divalent metal salt, including a counter anion which is not carbonate, such halide (e.g., chloride, fluoride, bromide, iodide), acetate, sulphate, nitrate, citrate, or any combination thereof) with a second aqueous composition comprising carbonate anions under suitable conditions, thereby obtaining the mixed particles of the invention.

In some embodiments, there is provided a method of manufacturing the mixed stabilized particles of the invention, the method comprising contacting a first aqueous composition comprising cations of the first divalent metal and of the second divalent metal (e.g. in a form of a dissociated divalent metal salt, including a counter anion which is not carbonate, such halide (e.g., chloride, fluoride, bromide, iodide), acetate, sulphate, nitrate, citrate, or any combination thereof) with a second aqueous composition comprising carbonate anions under suitable conditions, thereby obtaining the mixed stabilized particles of the invention, wherein the first aqueous composition and/or second aqueous composition further comprises the stabilizer dissolved or dispersed therewithin. In some embodiments, the first aqueous composition and/or second aqueous composition is/are substantially devoid of the stabilizer, and the method further comprising adding a stabilizer solution to the mixture subsequently or simultaneously with the contacting step.

In some embodiments, the method further comprising separating the particles from the aqueous solution, to obtain the mixed particles (and/or stabilized mixed particles) of the invention. In some embodiments, the method further comprising drying the particles under conditions sufficient for obtaining dry mixed particles (and/or stabilized mixed particles) of the invention, characterized by a water content as disclosed herein (up to 30% by weight).

In some embodiments, contacting is performed by mixing, stirring, shaking, or any other method known in the art. In some embodiments, suitable conditions comprise a temperature between 3 and 50° C., between 3 and 10° C., between 3 and 30° C., between 3 and 25° C., between 10 and 50° C., between 10 and 30° C., between 10 and 40° C., between 3 and 10° C., between 10 and 20° C., between 20 and 50° C., between 20 and 30° C., including any range between. In some embodiments, suitable conditions comprise a contacting time of at least 1 second, at least 1 minute, or more. In some embodiments, contacting time is sufficient for forming the particles of the invention. In some embodiments, contacting time is sufficient for reacting at least 50%, at least 60%, at least 80%, at least 90%, at least 95%, at least 99% including any range or value therebetween, or more of the initial divalent metal content within the first aqueous composition with the carbonate. In some embodiments, contacting time is sufficient for converting at least 50%, at least 60%, at least 80%, at least 90%, at least 95%, at least 99% including any range or value therebetween, or more of the initial divalent metal into the amorphous divalent metal carbonate (e.g. in the form of particles of the invention). In some embodiments, the initial divalent metal refers to the initial weight content of the first divalent metal and of the second divalent metal within the first aqueous composition.

Conversion can be monitored by any analytical technique known in the art (e.g. ICP, NMR, etc.). Furthermore, conversion can be monitored by monitoring the precipitation of the particles form the mixture. Thus, the end point of the particle formation step is indicated by a substantial arrest of the precipitation.

In some embodiments, the first aqueous composition and the second aqueous composition are aqueous solutions. In some embodiments, the first aqueous composition and the second aqueous composition comprise a sufficient amount of each the divalent metal and/or carbonate. In some embodiments, a concentration of the divalent metal cations (the first and the second divalent metals disclosed herein) within the first aqueous composition each independently is between 1 mM and 10M, is between 1 mM and 10 mM, is between 10 mM and 100 mM, is between 100 mM and 500 mM, is between 100 mM and 300 mM, is between 300 mM and 500 mM, is between 500 mM and 1M, including any range or value therebetween. In some embodiments, a concentration of carbonate within the second aqueous composition is between 1 mM and 10M, is between 1 mM and 10 mM, is between 10 mM and 100 mM, is between 100 mM and 500 mM, is between 100 mM and 300 mM, is between 300 mM and 500 mM, is between 500 mM and 1M, including any range or value therebetween.

In some embodiments, a molar ratio of the stabilizer relative to the divalent metal within the mixture is between 0.5 and 30%, between 0.5 and 10%, between 0.5 and 1%, between 1 and 30%, between 1 and 5%, between 5 and 10%, between 10 and 30%, at least 0.5%, at least 0.8%, at least 1%, at least 1.1%, at least 1.5%, at least 2%, at least 4%, at least 8%, including any range or value therebetween.

In some embodiments, a molar ratio of the carbonate relative to the divalent metal within the mixture is between about 0.5:1 and about 2:1, between about 0.5:1 and about 0.7:1, between about 0.7:1 and about 1:1, between about 1:1 and about 1:1.5, between about 1:1.5 and about 1:2, including any range or value therebetween.

In some embodiments, a molar ratio between the first second divalent metal and the second divalent metal within the first aqueous composition is between 10:0.05 and 1:1, between 10:0.05 and 1:0.1, between 10:0.1 and 1:0.2, between 10:0.2 and 1:1, between 10:0.2 and 1:0.1, between 10:0.2 and 1:0.5, between 10:0.2 and 10:0.3, between 10:0.3 and 10:2, between 10:0.3 and 10:0.4, between 10:0.4 and 10:0.5, between 10:0.5 and 10:0.7, between 10:0.7 and 10:0.8, between 10:0.8 and 10:1, between 10:1 and 10:2, including any range or value therebetween.

### Composition

In some embodiments, there is provided a composition comprising a plurality of particles of the invention (e.g. a single divalent metal particle, a mixed particle, a mixed stabilized particle, including any combinations thereof). In some embodiments, the composition substantially comprises a single particle specie or a plurality of chemically distinct particles. In some embodiments, the composition is a pharmaceutical composition comprising a therapeutically effective amount of the particles of the invention. In some embodiments, the composition is a pharmaceutical composition comprising a therapeutically effective amount of the amorphous divalent metal carbonate. In some embodiments, the composition is a nutraceutical composition comprising a nutraceutical effective amount of the particles of the invention. In some embodiments, the composition is a nutraceutical composition comprising a nutraceutical effective amount of the amorphous divalent metal carbonate.

In some embodiments, the composition of the present invention comprises the plurality of particles of the invention. In some embodiments, the plurality of particles of the invention are in a form of an agglomerate within the composition (e.g., powderous composition). In some embodiments, the plurality of particles of the invention are in a form of a particle cluster or agglomerate, also referred to herein, as a secondary particle.

In some embodiments, the secondary particle is stable, wherein stable is as described herein. In some embodiments, the secondary particle is in a form of a solid.

In some embodiments, the secondary particle is characterized by a particle size ranging from 0.1 to 500 μm, from 0.5 to 1 μm, from 1 to 2 μm, from 2 to 5 μm, from 0.1 to 10 μm, from 1 to 100 μm including any range between. In some embodiments, the powderous composition comprises a plurality of secondary particles.

In some embodiments, the powderous composition further comprises a non-bioactive or a non-therapeutic or nutritional additive or excipient (e.g., anti-caking agent, a desiccant, an antioxidant, a preservative etc.). In some embodiments, the powderous composition is a dry formulation. In some embodiments, the powderous composition is encapsulated (e.g., inside a capsule or a gel) for administration. In some embodiments, the powderous composition is pressed into tablets for administration. In some embodiments, the powderous composition is formulated in a suspension, a liquid or semiliquid for various administration including internal and topical uses. In some embodiments, the powderous composition is formulated in cell culturing media.

In some embodiments, a w/w concentration of the additives within the composition is between 0.01 and 20% including any range between.

In some embodiments, the water content of the powderous composition is less than 30, 20 or 10% w/w. In some embodiments, the water content of the powderous composition is less than 5% w/w. In some embodiments, the water content of the powderous composition is less than 2% w/w. In some embodiments, the water content of the powderous composition is less than 1% w/w.

In some embodiments, the composition of the invention comprises a plurality of particles of the invention, and an acceptable carrier. In some embodiments, the acceptable carrier is a pharmaceutically acceptable carrier. In some embodiments, the acceptable carrier is a nutraceutical acceptable carrier. In some embodiments, the acceptable carrier is a cell culturing acceptable carrier. In some embodiments, the acceptable carrier is a physiologically acceptable liquid. In some embodiments, the acceptable carrier is saline. In some embodiments, the acceptable carrier is serum.

In some embodiments, the w/w concentration of the carrier within the composition is between 1 and 99% including any range between.

In some embodiments, the composition is a formulation. In some embodiments, the composition is a suspension. The words “suspension” and “dispersion” are hereby interchangeable. In some embodiments, the particle (e.g., the primary particle and/or the secondary particle) is suspended in an aqueous solution. In some embodiments, the composition is a stable suspension. In some embodiments, the particle forms a stable dispersion in an aqueous solvent. In some embodiments, the stable dispersion is substantially devoid of a substantial agglomeration of the particles. In some embodiments, the stable dispersion is substantially devoid of precipitation of the particles for at least 10 min.

In some embodiments, the composition is an aqueous formulation, comprising the mixed stabilized particle of the invention. In some embodiments, the aqueous formulation comprising the mixed stabilized particles is stable for at least 48 h, at least 7 d, at least 1 month, at least 2 m, at least 3 m, at least 6 m, when stored at a temperature of less than 40° C., less than 30° C., less than 25° C., including any range between.

In some embodiments, the composition is an aqueous formulation, comprising the mixed particle of the invention. In some embodiments, the aqueous formulation comprising the stabilized particles is stable for at least 24 h, at least 48 h, at least 72 h, when stored at a temperature of less than 40° C., less than 30° C., less than 25° C., including any range between.

In some embodiments, the composition is an aqueous formulation, comprising a therapeutically effective amount of particles of the invention dispersed therewithin.

In some embodiments, the composition further comprises a polar solvent. In some embodiments, the polar solvent is a water miscible solvent.

In some embodiments, the acceptable carrier comprises water. In some embodiments, the acceptable carrier comprises an aqueous buffered solution.

In some embodiments, the composition is an aqueous composition (e.g., dispersion), wherein the aqueous composition is stable at a pH value of at least 7, at least 7.5, at least 8, including any range between.

In some embodiments, the aqueous composition comprises at least 70% w/w water. In some embodiments, the aqueous composition comprises at least 75% w/w water. In some embodiments, the aqueous composition comprises at least 85% w/w water. In some embodiments, the aqueous composition comprises at least 90% w/w water. In some embodiments, the aqueous composition comprises at least 95% w/w water. In some embodiments, the aqueous composition comprises at least 99% w/w water.

In some embodiments, the carrier is a physiologically acceptable carrier. In one embodiment, the phrases “physiologically acceptable carrier” and “pharmaceutically acceptable carrier” which be interchangeably used refer to a carrier or a diluent that does not cause significant irritation to an organism and does not abrogate the biological activity and properties of the administered compound. An adjuvant is included under these phrases. In one embodiment, “excipient” refers to an inert substance added to a pharmaceutical composition to further facilitate administration of an active ingredient. In one embodiment, excipients include calcium carbonate, calcium phosphate, various sugars and types of starch, cellulose derivatives, gelatin, vegetable oils and polyethylene glycols.

As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

In some embodiments, the composition is a nutraceutical composition. In some embodiments, the nutraceutical composition further comprises a food additive. Non-limiting examples of food additives include but are not limited to: flavonoids, carnitine, choline, vitamins, hydrophobic vitamins, polyunsaturated fatty acids, coenzyme Q, creatine, dithiolthiones, phytosterols, polysaccharides, nutraceuticals, antioxidants, phytoestrogens, glucosinolates, polyphenols, anthocyanins, or any combination thereof.

According to some embodiments, there is provided a method of supplementing a subject with an active agent, the method comprises administering to the subject a nutraceutically effective amount of the composition of the invention. In some embodiments, the active agent comprises the first divalent metal, the second divalent metal, or both. In some embodiments, the active agent comprises the carbonate specie. In some embodiments, the active agent comprises a first divalent metal carbonate, a second divalent metal carbonate, or both.

According to some embodiments, there is provided a method comprising modulating or affecting Cathepsin activity in a cell. In some embodiments, the method comprises contacting the cell with an effective amount of the particle disclosed herein, or a composition comprising same, as disclosed herein.

In some embodiments, the cell is a cell of a subject. In some embodiments, the cell is obtained or derived from a subject. In some embodiments, the cell is in the subject.

In some embodiments, the cell is in the subject and the contacting comprises administering to the subject a therapeutically effective amount of the particle disclosed herein, or a pharmaceutical composition comprising same, as disclosed herein.

In some embodiments, there is provided a method comprising treating, preventing, ameliorating, or any combination thereof, a Cathepsin-related or associated disease or disorder in subject in need thereof.

According to some embodiments, here is provided a pharmaceutical composition comprising the particles disclosed herein, for use in the treatment of a disease or a disorder.

In some embodiments, the disease or disorder is a Cathepsin-related or associated disease or disorder in subject in need thereof.

In some embodiments, a Cathepsin-related or associated disease or disorder comprises a cell proliferation related disease.

In some embodiments, a cell proliferation related disease comprises cancer.

In some embodiments, cancer comprises lung cancer.

In some embodiments, lung cancer comprises lung carcinoma.

As used herein, the term “Cathepsin-related or associated disease or disorder” encompasses any disease or disorder wherein Cathepsin expression and/or activity is involved, propagates, increases, results from, induces, participates, any combination thereof, or any equivalent thereof, in the pathogenesis and/or pathophysiology of the disease or disorder.

In some embodiments, expression comprises mRNA transcription, protein translation, or both.

In some embodiments, modulating or affecting comprises inhibiting, reducing, blocking, lowering, decreasing, any equivalent thereof, or any combination thereof, the activity of a Cathepsin. In some embodiments, Cathepsin comprises and acidophilic Cathepsin. In some embodiments, Cathepsin comprises a plurality of Cathepsins. According to some embodiment, the present invention provides a method of reducing activity of acidophilic Cathepsins in a subject in need thereof.

In some embodiments, the Cathepsin is selected from: B, K, A, G, C, F, H, L, V, W, X, D, E, or any combination thereof.

In some embodiments, the Cathepsin comprises or is Cathepsin B, Cathepsin K, or both.

In some embodiments, treating comprises reducing activity of the acidophilic Cathepsin in a subject, or in a cell obtained or derived therefrom.

As used herein, the term “Cathepsin” refers to a protein belonging to a group of lysosomal proteases that have a key role in cellular protein turnover. As used herein, the term “Cathepsin” encompasses serine proteases, aspartic proteases, and cysteine proteases. Based on their catalytic mechanism, Cathepsins are subdivided into serine (A and G), cysteine (B, C, F, H, K, L, O, S, V, W and X) and aspartic proteases (D and E). Most of the Cathepsins are acidophilic Cathepsins, e.g., are most active in a slightly acidic to acidic environment. An exception is Cathepsin S which is a non-acidophilic Cathepsin being active under physiological conditions and even under slightly alkaline conditions. According to some embodiments, the Cathepsins are human Cathepsins.

According to some embodiments, the method comprises reducing activity of Cathepsin B. According to other embodiment, the method comprises reducing activity of Cathepsin K. According to another embodiments, the method comprises reducing activity of Cathepsin B and K.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, provide improvement to a patient or subject's quality of life, or reduce the chances of infectivity of the patient or from the patient to others.

As used herein, the term “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described compositions or composition prior to the induction or onset of the disease/disorder process. The term “suppression” is used to describe a condition wherein the disease/disorder process has already begun but obvious symptoms of the condition have yet to be realized. Thus, the cells of an individual may have the disease/disorder, but no outside signs of the disease/disorder have yet been clinically recognized. In either case, the term prophylaxis can be applied to encompass both prevention and suppression. Conversely, the term “treatment” refers to the clinical application of active agents to combat an already existing condition whose clinical presentation has already been realized in a patient.

As used herein, “treating” comprises ameliorating and/or preventing.

In some embodiments, ameliorating comprises alleviating at least one symptom associated with a disease as described herein.

As used herein, the terms “administering”, “administration” and like terms refer to any method which, in sound medical practice, delivers a composition containing an active agent to a subject in such a manner as to provide a therapeutic effect.

As used herein, the terms “subject” or “individual” or “animal” or “patient” or “mammal” refers to any subject, particularly a mammalian subject, for whom therapy is desired, for example, a human.

In some embodiments, a therapeutically effective dose of the composition of the invention is administered. The term “therapeutically effective amount” refers to an amount of a drug effective to treat a disease or disorder in a mammal. The term “a therapeutically effective amount” refers to an amount effective, at dosages and for periods of time necessary, to achieve the desired therapeutic or prophylactic result. The exact dosage form and regimen would be determined by the physician according to the patient's condition.

In some embodiments, a nutraceutically effective dose of the composition of the invention is administered. The term “nutraceutically effective amount” refers to an amount of the active agent effective for supplementing the subject with the active agent. The term “a therapeutically effective amount” refers to an amount effective, at dosages and for periods of time necessary, to enhance the concentration, optionally so as to achieve a predetermined concentration of the active agent within the subject's body. The exact dosage may vary dependent of the desired predetermined concentration of the active agent and on the initial amount of the active agent within the organism.

The dosage administered will be dependent upon the age, health, and weight of the recipient, kind of concurrent treatment, if any, frequency of treatment, and the nature of the effect desired. The route of administration of the pharmaceutical compositions will depend on the disease or condition to be treated. Suitable routes of administration include, but are not limited to, parenteral injections, e.g., intradermal, intravenous, intramuscular, intralesional, subcutaneous, intrathecal, and any other mode of injection as known in the art. Although the bioavailability of peptides administered by other routes can be lower than when administered via parenteral injection, by using appropriate compositions it is envisaged that it will be possible to administer the compositions of the invention via transdermal, oral, rectal, vaginal, topical, nasal, inhalation and ocular modes of treatment. In addition, it may be desirable to introduce the pharmaceutical compositions of the invention by any suitable route, including intraventricular and intrathecal injection; intraventricular injection may be facilitated by an intraventricular catheter, for example, attached to a reservoir.

In some embodiments, the composition is delivered orally. In some embodiments, the composition is an oral composition. In some embodiments, the composition further comprises orally acceptable carrier, excipient, or a diluent. In some embodiments, the composition is formulated for oral delivery.

### General

As used herein the term “about” refers to ±10%.

The terms “comprises”, “comprising”, “includes”, “including”, “having” and their conjugates mean “including but not limited to”. The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

As used herein, the term “substantially” refers to at least 80%, at least 85%, at least 90%, at least 92%, at least 95%, at least 96%, at least 97%, at least 98%, at least 99%, including any range or value therebetween.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein, the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Other terms as used herein are meant to be defined by their well-known meanings in the art.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

### Materials and Methods

Preparation of Amorphous Zinc Carbonate (AZC); 1% Zn Suspension Optionally Stabilized with 10% TP

In a typical procedure, a Zinc chloride solution (100 ml of water, 5.21 g of zinc chloride, 38.2 mmol) a sodium carbonate solution (100 ml of water, 4.05 g of sodium carbonate, 38.2 mmol) and a sodium tripolyphosphate (STPP) solution (50 ml of water, g of sodium tripolyphosphate, 1.4 mmol) were mixed at room temperature using a stirrer in the aim to precipitate amorphous zinc carbonate (AZC) stabilized with tripolyphosphate. The mixing of the solutions was performed in the following order: at first the STPP solution (described above in Example 1) was divided into two equal parts of (25 ml of water, 0.27 g of Sodium tripolyphosphate) each. One part was added to a zinc chloride solution with stirring, so as to form AZC nanoparticles; whilst the second part of STPP solution was added after the formation of the AZC nanoparticles, resulting in a AZC-STPP stabilized milky suspension. The AZC was filtered using a Buchner, washed with ethanol in abundant quantities, and dried, by oven with a ventilation valve at 100° C. for not more than 30 minutes to obtain a powder in 2.7 g yield.

AZC with varied percentages of STTP were synthesized according to the same procedure in order to identify optimal levels of the stabilizers. The AZCs were filtered using a Buchner, washed with ethanol in abundant quantities, and dried, by oven, with a ventilation valve at 100° C. for not more than 30 minutes to obtain a powder in 4.4 g yield. The ethanol was used as an efficient means to rapidly dry the evolved amorphous phases in this series of examples. However, other modes of drying such products for achieving storable powders at large scale are feasible.

The powders obtained according to this procedure were analyzed using X-Ray diffraction, ICP, TGA, DSC, SEM. Both, suspensions and selected dry powders were analyzed for their short-term (suspensions) and long-term (powders) stability. The suspension stability is also a good approach to screen and assess the evolved amorphous phases within a short duration. Suspensions were also tested for their native pH level and the stability of the suspension (i.e., the time that takes the particulates to settle at the bottom of the container and if there is a fraction of particles that remain suspended over time.

Alternatively, amorphous zinc carbonate was prepared according to the method described hereinabove without the addition of a stabilizer.

Exemplary methods for preparation of mixed amorphous metal carbonates and metal inclusion in ACC, stabilized by sodium tripolyphosphate (STPP)

A: Incorporation of a Second Metal and a Stabilizer During and after the Synthesis of ACC

In a typical procedure consists of mixing (1) calcium chloride dihydrate solution (80 ml of water, 7.35 g of calcium chloride dihydrate; 50.0 mmol); (2) sodium carbonate solution (80 ml of water, 5.3 g of sodium carbonate; 50.0 mmol); and optionally (3) a typical Stabilizer, which is a sodium tripolyphosphate (STPP) solution (0.74 g of sodium tripolyphosphate; 2.0 mmol; 10% w/w of the CaCl₂.2H₂O); (4) a second metal (M) solution, which was added at different concentrations at various weight ratios of M/Ca reagents. Most metals were added as hydrate chloride compounds except Fe that was added as FeSO₄. For example, ACC containing Zn, Mg and Fe in Ca carbonate were synthesized with various reagents' weight fractions of M/Ca by w/w and other ratios are given by w/w compared to the calcium reagent content.

One way of mixing the reaction solutions was as follows. The stabilizer was added as a mixture with the second metal, and the final volume of the mixed solution was divided into two equal parts. The first part was added to the calcium chloride dihydrate solution before mixing with the sodium carbonate solution, and the second part was added after adding the sodium carbonate solution to the stirred calcium chloride dihydrate solution and after the precipitation of the nanoparticle agglomerates. The final volume of mixed solutions was adjusted to attain a 0.45%, 1% or other concentrations of calcium in the suspension, calculated by w/v. For low fractions of the second metal (up to 10 wt %) the carbonate ions were present in equimolar quantity compared to the calcium ions.

The Stabilized M-ACC's were filtered using a Buchner, washed with ethanol in abundant quantities, and dried, by oven with a ventilation valve at 100° C. for no longer than 30 minutes to obtain a powder in the range of 4 to 5 g yield. For larger scaled up production quantities, the ethanol washing is eliminated. However, other modes of drying such products for achieving storable powders at large scale are feasible.

The powders obtained according to this procedure were analyzed using X-Ray diffraction, ICP, TGA, DSC, SEM. Both, suspensions and selected dry powders were analyzed for their short-term (suspensions) and long-term (powders) stability. The suspension stability is also a good approach to screen and assess the evolved amorphous phases within a short duration. Suspensions were also tested for their native pH level and the stability of the suspension (i.e., the time that takes the particulates to settle at the bottom of the container and if there is a fraction of particles that remain suspended over time.

### B: Incorporation of a Second Metal During the Formation of the ACC Nanoparticles

The same solutions as in A were prepared. However, in this case the second metal solution was completely added to the calcium chloride solution before any addition of other reagent solutions. Then, the sodium carbonate solution was rapidly added to the metal solution, followed by immediate addition of the stabilizer (e.g., STPP) as the last solution to be added to the reaction solution. The isolation, drying and analyses of the obtained metal carbonate powders were performed as in A.

C: Incorporation of a Second Metal after the Formation of ACC Nanoparticles

The same solutions as in A were prepared. However, in this case the stabilizer solution was added first to the calcium chloride solution. Then, the sodium carbonate solution was rapidly added to the first solution, followed by immediate addition of the second metal solution as the last solution to be added to the reaction solution. The isolation, drying and analyses of the obtained metal carbonate powders were performed as in A.

D: Incorporation of a Second Metal Before the Formation of ACC and a Stabilizer being Added During and after the Formation

The same solutions as in A were prepared. However, in this case the second metal solution was completely added to the calcium chloride solution, before adding any other reagent solutions. Then, half of the stabilizer was added to the mixed metal solution. In the next step, the sodium carbonate solution was rapidly added to the metal solution so as to result in the formation of mixed particles (containing ACC and the second metal), followed by immediate addition of the second half of the stabilizer solution (e.g., STPP). The isolation, drying and analyses of the obtained metal carbonate powders were performed as in A.

Furthermore, non-stabilized mixed metal carbonates were prepared according to the method described hereinabove, without the addition of a stabilizer.

It should be apparent, that for the synthesis of the amorphous particles of the invention calcium chloride, disclosed in A (or any other metal chloride), can be replaced by any appropriate water-soluble metal salt such as metal sulphate and/or metal nitrate. Additionally, any carbonate source can be utilized, such as Na-, K-, and/or NH₄-carbonates and organo-carbonates such as dimethyl carbonate, etc.

### Example 1

### Analysis of Main Elements of Synthesized ACC, AMC, AZC, and M-ACC

Table 1 summarizes the Ca:M:P mole ratio based on the as-introduced quantities of the reagents used in the materials and methods described above and compare these ratios to the actual ratios analyzed in the final dry amorphous powder, using inductively Coupled Plasma (ICP) analysis. Most of the obtained amorphous materials are stabilized with triphosphate as the stabilizing agent, in addition to the somewhat stabilization provided by inclusions of other metals into ACC framework.

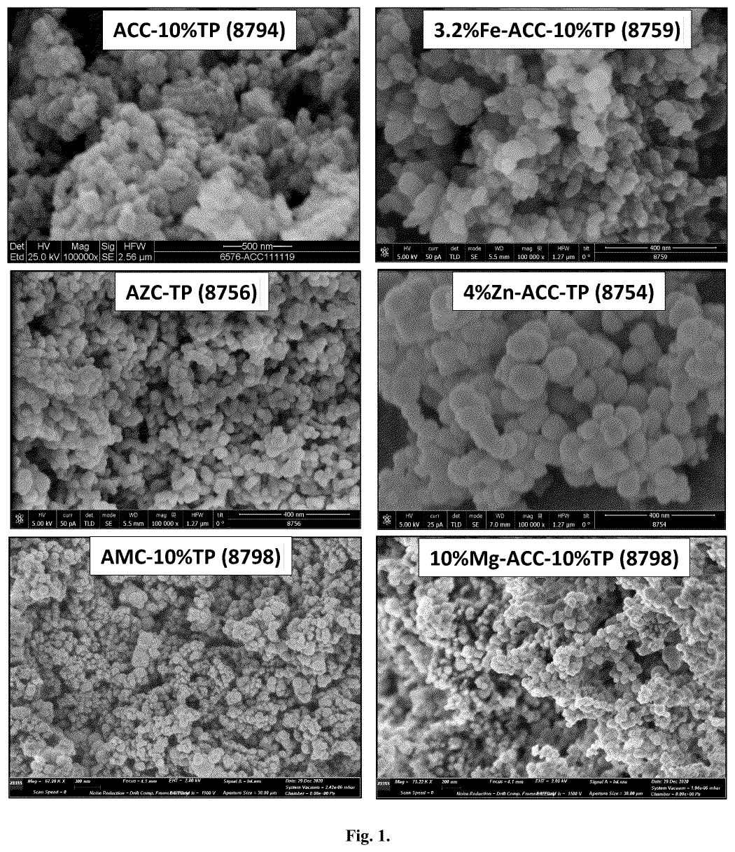

FIG. 1 presents scanning electron microscopy (SEM) images of amorphous metal carbonate nanoparticle clusters, obtained according to any of the procedures described hereinabove. The overall pattern consists of primary nanoparticles, aggregated/agglomerated into large cluster particles, with various degrees of necking between the nanoparticles (secondary particles). The nanoparticle size range is between to 100 nm.

TABLE 1
Elemental analysis versus calculated values of amorphous metal and mixed metal
carbonates stabilized and non-stabilized. The way to decode the synthesis
name: “1% Ca-6% w/w Zn(int)/Ca-10% TP(ext)” means 1% total calcium
in the suspension; the metal (zinc) fraction compared to calcium by weight
is 6%; the stabilizer fraction (TP) compared to calcium by weight is 10%;
the Zn solution was added in the initial reaction and the TP was added after
the precipitation. If int. or ext. are not mentioned, then the metal solution
was added to the calcium solution first and the stabilizer was added half
during the formation of the carbonate and half after the initial precipitation.

| | | Wt % | Wt % | | Ca:M:P |
|---|---|---|---|---|---|
| | | Ca | metal | Wt % P | mole ratio |
| Exp. # | Synthesis name* | (analysis) | (analysis) | (analysis) | (analysis) |
| 8794 | ACC-TP: 0.45% Ca- | 29.76 | 0 | 2.98 | 1:0.00:0.12 |
| | 10% TP | | | | |
| 8751 | Fe-ACC: 1% Ca-2% | 33.10 | Fe | 3.21 | 1:0.017:0.13 |
| | w/w Fe/Ca-10% TP | | 0.78 | | |
| 8759 | Fe-ACC: 1% Ca-4% | 31.20 | Fe | 3.13 | 1:0.033:0.13 |
| | w/w Fe/Ca-10% TP | | 1.42 | | |
| 8803 | Fe-ACC: 0.45% Ca-5% | 30.70 | Fe | 2.77 | 1:0.039:0.12 |
| | w/w Fe/Ca-10% TP | | 1.58 | | |
| 8804 | Fe-ACC: 0.45% Ca-10% | 29.20 | Fe | 2.81 | 1:0.075:0.12 |
| | w/w Fe/Ca -10% TP | | 3.07 | | |
| 8805 | Fe-ACC: 0.45% Ca-10% | 32.3 | Fe | 0 | 1:0.079:0 |
| | w/w Fe/Ca-0% TP | | 3.56 | | |
| 8756 | AZC: 1% Zn -10% TP | N/A | Zn | 3.20 | 0:1:0.0.10 |
| | | | 54.80 | | |
| 8752 | Zn-ACC: 1% Ca-2% | 34.40 | Zn | 3.30 | 1:0.016:0.12 |
| | w/w Zn(int)/Ca- | | 0.92 | | |
| | 10% TP(ext) | | | | |
| 8757 | Zn-ACC: 1% Ca-2% | 31.10 | Zn | 3.12 | 1:0.020:0.13 |
| | w/w Zn(ext)/Ca- | | 1.00 | | |
| | 10% TP(int) | | | | |
| 8754 | Zn-ACC: 1% Ca-6% | 30.50 | Zn | 2.99 | 1:0.040:0.13 |
| | w/w Zn(int)/Ca- | | 2.03 | | |
| | 10% TP(ext) | | | | |
| 8755 | ACC-Synthesis- | 30.70 | Zn | 3.04 | 1:0.040:0.13 |
| | 251120-1% Ca-6% w/w | | 2.00 | | |
| | Zn/Ca-TP10% | | | | |
| 8758 | ACC-Synthesis- | 29.56 | Zn | 2.99 | 1:0.042:0.13 |
| | 291120-1% Ca-6% w/w | | 2.00 | | |
| | Zn/Ca-TP10% | | | | |
| 8799 | Zn-ACC: 0.45% Ca- | 30.7 | Zn | 2.64 | 1:0.029:0.11 |
| | 5% w/w Zn/Ca-10% TP | | 1.42 | | |
| 8800 | Zn-ACC: 0.45% Ca- | 30.0 | Zn | 2.74 | 1:0.047:0.09 |
| | 10% w/w Zn/Ca-10% TP | | 3.06 | | |
| 8801 | Zn-ACC: 0.45% Ca- | 31.95 | Zn | 0 | 1:0.068:0 |
| | 10% w/w Zn/Ca -0% TP | | 3.57 | | |
| 8795 | AMC: 1% Mg-10% TP | 0 | Mg | 2.88 | 0:1:0.09 |
| | | | 24.7 | | |
| 8796 | Mg-ACC: 0.45% Ca- | 30.9 | Mg | 2.3 | 1:0.027:0.10 |
| | 5% w/w Mg/Ca-10% TP | | 0.52 | | |
| 8797 | Mg-ACC: 0.45% Ca- | 29.9 | Mg | 2.38 | 1:0.064:0.11 |
| | 10% w/w Mg/Ca- | | 1.18 | | |
| | 10% TP | | | | |
| 8798 | Mg-ACC: 0.45% Ca- | 33.1 | Mg | 0 | 1:0.043:0 |
| | 10% w/w Mg/Ca-0% TP | | 0.87 | | |

*Nomenclature of experiments: ACC = amorphous calcium carbonate, AMC = amorphous magnesium carbonate, AZC = amorphous zinc carbonate; stabilizers: TP = triphosphate, PS = phosphoserine, CA = citric acid; int = addition of reagent during the initial mixing of the calcium and carbonate solutions, ext = addition of the reagent after the initial ACC precipitation; w/w = weight-to-weight ratio; X % TP or another stabilizer means the weight % of sodium tripolyphosphate compared to the metal reagent weight.

Furthermore, the long-term stability of the synthesized amorphous metal carbonates (in a form of a dry powder) has been evaluated, which were stored under dry conditions and at 25° C. (except cases in which they were incubated at 40° C.). The results are summarized in Table 1A below.

| | Amorphous metal carbonate | No of months | Stability by |
|---|---|---|---|
| # | Composition | from Synthesis | XRD |
| 1 | ACC-Zn 10% TP-10% w/w Zn/Ca | 6 M | 100 |
| 2 | ACC 0% TP-10% w/w Zn/Ca | 6 M | 100 |
| 3 | AZC- Zn carbonate 10% TP | 6 M | 100 |
| 4 | ACC-Mg 10% TP-10% w/w Mg/Ca | 7 M | 100 |
| 5 | ACC-Mg 10% TP-10% w/w Mg/Ca- | 7 M | 99 |
| 6 | ACC-Fe 10% TP-2% w/w Fe/Ca | 3 M | 100 |
| 7 | ACC-Zn10% TP(Ext)-2% w/w Zn/Ca | 3 M | 60 |
| 8 | ACC-Zn 10% TP(Ext)-6% w/w Zn/Ca | 3 M | 100 |
| 9 | ACC-Zn(Ext) 10% TP(Int)-6% w/w Zn/Ca | 3 M | 88 |
| 10 | ACC-Zn(Ext) 10% TP(Int)-2% w/w Zn/Ca | 3 M | 100 |
| 11 | ACC- Zn 10% TP-6% w/w Zn/Ca | 3 M | 100 |
| 12 | ACC-Fe(II) 10% TP-2% w/w Fe/Ca | 6 M | 94 |
| 13 | ACC- Zn(Int) 10% TP(Ext)-2% w/w Zn/Ca | 6 M | 33 |
| 14 | ACC- Zn(Int) 10% TP(Ext)-6% w/w Zn/Ca | 6 M | 100 |
| 15 | ACC- Zn(Ext) 10% TP(Int)-6% w/w Zn/Ca | 6 M | 84 |
| 16 | ACC- Zn(Ext) 10% TP(Int)-2% w/w Zn/Ca | 6 M | 98 |
| 17 | ACC- Zn 10% TP-6% w/w Zn/Ca | 6 M | 95 |
| 18 | ACC-Fe(II) 10% TP-4% w/w Fe/Ca | 6 M | 95 |
| 19 | AMC Mg carbonate % TP (25C) | 3 M | 99 |
| 20 | AMC Mg carbonate % TP (40C) | 3 M | 99 |

Legend: ACC = amorphous calcium carbonate; AMC = amorphous magnesium, carbonate; AZC = amorphous zinc carbonate; TP = polytriphosphate; int = incorporation of the metal or the stabilizer during the initial synthesis step; ext = adding the second or more metal or the stabilizer after the initial precipitation of the ACC.

The long-term stability of additional amorphous metal carbonates (i.e. single divalent particles of the invention) and mixed metal carbonates (i.e. mixed particles and mixed stabilized particles) that were stored in the form of “dry” powders (typically, containing 8 to 15 wt % water) at room temperature for periods of months has been evaluated based on XRD analysis.

To this end, the inventors demonstrated successful synthesis and long term stability (at least 3 month, and up to 12 months or more) of (i) single divalent particles of the invention: AZC, AMC with about 10% w/w of stabilizer (relative to the metal); and (ii) of mixed and stabilized mixed divalent particles of the invention: Ca/Zn [2-10% w/w Zn relative to Ca] with stabilizer, Zn located in the interior or at the exterior of the particle; Ca/Zn [about 10-20% w/w Zn] without stabilizer; Ca/Mg (10-20% w/w Mg relative to Ca) without stabilizer. Ca/Mg (5-10% w/w Mg relative to Ca) with about 10% w/w of stabilizer; Ca/Fe(II) with stabilizer (about 2-4% w/w Fe relative to Ca); Ca/Fe(II) without stabilizer (10-20% w/w Fe relative to Ca).

### Example 2

### Compositions of Exemplary Amorphous Metal and Mixed-Metal Carbonates

Table 2 summarizes the compositions of amorphous metal and mixed metal carbonates obtained in the materials and methods section. The approximate analysis of the materials' compositions is based on the combined ICP analyses and thermal gravimetric analyses (TGA). Typical TGAs of these materials indicate ranges of temperatures in which weight loss is associated with (a) lightly adsorbed water; (b) tightly absorbed water (complexed to the molecular structure and/or trapped in nanometric pores); (c) water evolved during high temperature condensations of dangling hydroxyl groups (M-O—H, P—OH and C—O—H) in which 2 groups of —OH condense to form M-O-M, M-O—P or M-O—C bonding and a molecule of water is released; and (d) release of CO₂ by the high temperature decomposition of the carbonate to metal oxide and released CO₂.

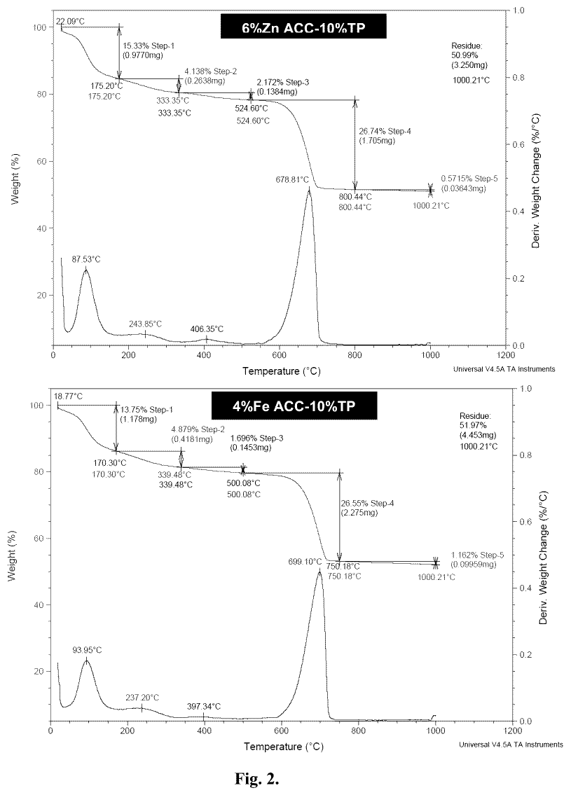

FIG. 2 represents 2 examples of such typical TGA's. It should be noted that since any of the above ranges are wide, they are overlapping in their chemical activities. Hence, the assigned weigh losses in a certain range. AS represented in FIG. 2, TGA of M-ACC-TP reveals 4 significant regions of weight loss at the following maximum rate temperature: ˜90° C. (loss of adsorbed water); ˜240° C. (loss of complexed and trapped water); ˜400° C. (water due to condensation of —OH groups); ˜68° C. (CO₂ due to carbonate decomposition).

TABLE 2
Compositions of amorphous metal and mixed metal carbonates obtained in
the materials and methods based on ICP and TGA analyses.

| | | | | | | | | Wt % | |
|---|---|---|---|---|---|---|---|---|---|
| | | | | | | | | Carbonate | |
| | | | | | | | | (TGA; | |
| | | | | | | | | Calculated | Approximate |
| | | | | | | | Wt % | from | Composition |
| | | | | Wt % P | | | OH | CO₂ | CaM_{b}(CO₃)_{c} |
| | | Wt % | Wt % | (ICP) | | Complexed | (TGA; | release | (P₃O₁₀)_{d} |
| | | Ca | M | Calculated | Wt % | and | calculated | and | (OH)ₑ (OH |
| | | (ICP) | (ICP) | Phosphate | Adsorbed | trapped | based or | conversion | can be M—OH, |
| | Synthesis | Mol | Mol | Mol | Water | water | condensation | to | P—O, C—OH) |
| Exp# | Name | fraction | fraction | fraction | (TGA) | (TGA) | reaction | CO₃) | (H²O)^{f} |
| 8794 | ACC-TP: | Ca | N/A | P 2.98 | 13.3 | 3.8 | 1.6 | 29.0 | Ca_{1.00}(CO₃)_{0.88} |
| | 0.45% | 29.76 | | P → | | Mol | H₂O → | CO₂ → | (P₃O₁₀)_{0.043} |
| | Ca- | Mol | | (P₃O₁₀)/3 | | fraction | 2OH | CO₃ | Overall OH |
| | 10% | fraction | | 2.98 × 253/ | | 3.8/18 = | 1.6 × 34/ | 29.0 × | groups |
| | TP | 29.8/ | | 31/3 = 8.11 | | 0.21 | 18 = 3.0 | 60/44 = | (OH)_{0.24} |
| | | 40 = 0.75 | | Mol | | | Mol | 39.5 | Adduct and |
| | | | | fraction | | | fraction | Mol | trapped |
| | | | | 8.11/253 = | | | 3.0/17 = | fraction | internal |
| | | | | 0.032 | | | 0.18 | 39.5/60 = | water |
| | | | | | | | | 0.66 | (H₂O)_{0.28} |
| 8751 | Fe- | Ca | Fe 0.80 | P 3.21 | 7.7 | 4.9 | 1.5 | 29.1 | Ca_{1.00}Fe_{0.017} |
| | ACC: | 33.1 | Mol | P → | | Mol | H₂O | CO₂ → | (CO₃)_{0.80} |
| | 1% Ca- | Mol | fraction | (P₃O₁₀)/ | | fraction | 2OH | CO₃ | (P₃O₁₀)_{0.035} |
| | 2% | fraction | 0.8/ | 33.21 × | | 4.9/18 = | 1.5 × 34/ | 29.1 × 60/ | Overall OH |
| | w/w | 33.1/ | 55.9 = | 253/31/ | | 0.27 | 18 = 2.83 | 44 = 39.5 | groups |
| | Fe/Ca- | 40 = 0.82 | 0.014 | 3 = 8.73 | | | Mol | Mol | (OH)_{0.21} |
| | 10% | | | Mol | | | fraction | fraction | Adduct and |
| | TP | | | fraction | | | 2.83/17 = | 39.5/60 = | trapped |
| | | | | 8.73/253 = | | | 0.17 | 0.66 | internal |
| | | | | 0.035 | | | | | water |
| | | | | | | | | | (H₂O)_{0.33} |
| 8759 | Fe- | Ca | Fe 1.42 | P 3.13 | 13.8 | 4.9 | 1.7 | 26.6 | Ca_{1.00}Fe_{0.032} |
| | ACC: | 31.2 | Mol | P → | | Mol | H₂O → | CO₂ → | (CO₃)_{0.78} |
| | 1% Ca- | Mol | fraction | (P₃O₁₀)/ | | fraction | 2OH | CO₃ | (P₃O₁₀)_{0.043} |
| | 4% | fraction | 1.42/55 | 33.13 × | | 4.9/18 = | 1.7 × 34/ | 26.6 × 60/ | Overall OH |
| | w/w | 31.2/ | .9 = | 253/31/3 = | | 0.27 | 18 = 3.21 | 44 = 36.3 | groups |
| | Fe/Ca- | 40 = 0.78 | 0.025 | 8.51 | | | Mol | Mol | (OH)_{0.23} |
| | 10% | | | Mol | | | fraction | fraction | Adduct and |
| | TP | | | fraction | | | 3.21/17 = | 36.3/60 = | trapped |
| | | | | 8.51/253 = | | | 0.19 | 0.60 | internal |
| | | | | 0.034 | | | | | water |
| | | | | | | | | | (H₂O)_{0.33} |
| 8803 | Fe- | Ca | Fe 1.58 | P 2.77 | 8.33 | 4.41 | 1.48 | 27.2 | Ca_{1.00}Fe_{0.039} |
| | ACC: | 30.7 | Mol | P → | | Mol | H₂O → | CO₂ → | (CO₃)_{0.79} |
| | 0.45% | Mol | fraction | (P₃O₁₀)/ | | fraction | 2OH | CO₃ | (P₃O₁₀)_{0.043} |
| | Ca-5% | fraction | 1.58/ | 32.77 × | | 4.4/18 = | 1.48 × 34/ | 27.2 × | Overall OH |
| | w/w | 30.7/ | 55.9 = | 253/31/3 = | | 0.24 | 18 = 2.80 | 60/44 = | groups |
| | Fe/Ca- | 40 = 0.77 | 0.028 | 7.54 | | | Mol | 37.1 | (OH)_{0.21} |
| | 10% | | | Mol | | | fraction | Mol | Adduct and |
| | TP | | | fraction | | | 3.21/17 = | fraction | trapped |
| | | | | 7.64/253 = | | | 0.16 | 36.3/ | internal |
| | | | | 0.030 | | | | 60 = | water |
| | | | | | | | | 0.62 | (H₂O)_{0.31} |
| 8804 | Fe- | Ca | Fe 3.07 | P 2.81 | 9.1 | 4.22 | 1.48 | 25.8 | Ca_{1.00}Fe_{0.075} |
| | ACC: | 29.2 | Mol | P | | Mol | H₂O → | CO₂ → | (CO₃)_{0.81} |
| | 0.45% | Mol | fraction | (P₃O₁₀)/ | | fraction | 2OH | CO₃ | (P₃O₁₀)_{0.041} |
| | Ca-10% | fraction | 3.07/ | 32.81 × | | 4.22/18 = | 1.48 × 34/ | 25.8 × | Overall OH |
| | w/w | 29.2/ | 55.9 = | 253/31/3 = | | 0.23 | 18 = 2.80 | 60/44 = | groups |
| | Fe/Ca- | 40 = 0.73 | 0.055 | 7.64 | | | Mol | 35.1 | (OH)_{0.22} |
| | 10% | | | Mol | | | fraction | Mol | Adduct and |
| | TP | | | fraction | | | 3.21/17 = | fraction | trapped |
| | | | | 7.64/253 = | | | 0.16 | 36.3/ | internal |
| | | | | 0.030 | | | | 60 = | water |
| | | | | | | | | 0.59 | (H₂O)_{0.32} |
| 8805 | Fe- | Ca | Fe 3.56 | P 0% | 5.24 | 4.22 | 0.5 | 33.6 | Ca_{1.00}Fe_{0.079} |
| | ACC: | 32.2 | Mol | | | Mol | H₂O → | CO₂ → | (CO₃)_{0.94} |
| | 0.45% | Mol | fraction | | | fraction | 2OH | CO₃ | Overall OH |
| | Ca-10% | fraction | 3.56/ | | | 4.13/18 = | 0.5 × 34/ | 33.6 × | groups |
| | w/w | 32.3/ | 55.9 = | | | 0.23 | 18 = 2.80 | 60/44 = | (OH)_{0.07} |
| | Fe/Ca- | 40 = 0.81 | 0.064 | | | | Mol | 45.8 | Adduct and |
| | 0% | | | | | | fraction | Mol | trapped |
| | TP | | | | | | 0.94/17 = | fraction | internal |
| | | | | | | | 0.06 | 36.3/60 = | water |
| | | | | | | | | 0.76 | (H₂O)_{0.28} |
| 8756 = | AZC: | Zn | N/A | P 3.20 | 4.5 | 4.5 | 9.0 | 11.6 | Zn_{1.00}(CO₃)_{0.26} |
| 8802 | 1% Zn- | 54.8 | | P → | | Mol | H₂O → | CO₂ → | (P₃O₁₀)_{0.034} |
| | 10% | Mol | | (P₃O₁₀)/ | | fraction | 2OH | CO₃ | Overall OH |
| | TP | fraction | | 33.2 × | | 4.5/18 = | 9.0 × 34/ | 11.6 × | groups |
| | | 54.8/ | | 253/31/3 = | | 0.25 | 18 = 17 | 60/44 = | (OH)_{1.22} |
| | | 65.4 = | | 8.71 | | | Mol | 15.8 | Adduct and |
| | | 0.84 | | Mol | | | fraction | Mol | trapped |
| | | | | fraction | | | 17/17 = | fraction | internal |
| | | | | 8.71/253 = | | | 1.00 | 15.8/60 = | water |
| | | | | 0.034 | | | | 0.26 | (H₂O)_{0.30} |
| | | | | | | | | | Possibly, |
| | | | | | | | | | Some of the |
| | | | | | | | | | OH weight |
| | | | | | | | | | loss is |
| | | | | | | | | | attributed to |
| | | | | | | | | | the loss of |
| | | | | | | | | | CO₂ via the |
| | | | | | | | | | direct |
| | | | | | | | | | reaction with |
| | | | | | | | | | OH |
| 8752 | Zn- | Ca | Zn 0.92 | P 3.30 | 9.5 | 2.0 | 2.0 | 29.9 | Ca_{1.00}Zn_{0.014} |
| | ACC: | 34.4 | Mol | P → | | Mol | H₂O → | CO₂ → | (CO₃)_{0.79} |
| | 1% Ca- | Mol | fraction | (P₃O₁₀)/ | | fraction | 2OH | CO₃ | (P₃O₁₀)_{0.035} |
| | 2% | fraction | 0.92/ | 33.30 × | | 2.0/18 = | 2.0 × 34/ | 29.9 × | Overall OH |
| | w/w | 34.4/ | 65.4 = | 253/31/3 = | | 0.11 | 18 = 3.77 | 60/44 = | groups |
| | Zn | 40 = 0.86 | 0.014 | 8.98 | | | Mol | 40.8 | (OH)_{0.26} |
| | (int)/Ca- | | | Mol | | | fraction | Mol | Adduct and |
| | 10% | | | fraction | | | 3.77/17 = | fraction | trapped |
| | TP | | | 8.98/253 = | | | 0.22 | 40.8/60 = | internal |
| | (ext) | | | 0.035 | | | | 0.68 | water |
| | | | | | | | | | (H₂O)_{0.13} |
| 8757 | Zn- | Ca | Zn 0.99 | P 3.12 | 15.34 | 4.7 | 1.5 | 26.7 | Ca_{1.00}Zn_{0.019} |
| | ACC: | 31.1 | Mol | P → | | Mol | H₂O → | CO₂ → | (CO₃)_{0.78} |
| | 1% Ca- | Mol | fraction | (P₃O₁₀)/ | | fraction | 2OH | CO₃ | (P₃O₁₀)_{0.044} |
| | 2% | fraction | 0.99/ | 33.12 × | | 4.7/18 = | 1.5 × 34/ | 26.7 × | Overall OH |
| | w/w | 31.1/ | 65.4 = | 253/31/3 = | | 0.26 | 18 = 2.83 | 60/44 = | groups |
| | Zn | 40 = 0.78 | 0.015 | 8.49 | | | Mol | 36.4 | (OH)_{0.22} |
| | (ext)/Ca- | | | Mol | | | fraction | Mol | Adduct and |
| | 10% | | | fraction | | | 2.83/17 = | fraction | trapped |
| | TP | | | 8.49/253 = | | | 0.17 | 36.4/60 = | internal |
| | (int) | | | 0.034 | | | | 0.61 | water |
| | | | | | | | | | (H₂O)_{0.33} |
| 8754 | Zn- | Ca | Zn 2.03 | P 2.99 | 15.3 | 4.1 | 2.2 | 26.7 | Ca_{1.00}Zn_{0.04} |
| | ACC: | 30.5 | Mol | P → | | Mol | H₂O → | CO₂ → | (CO₃)_{0.80} |
| | 1% Ca- | Mol | fraction | (P₃O₁₀)/ | | fraction | 2OH | CO₃ | (P₃O₁₀)_{0.042} |
| | 6% | fraction | 2.03/ | 32.99 × | | 4.10/18 = | 2.2 × 34/ | 26.7 × | Overall OH |
| | w/w | 30.5/ | 65.4= | 253/31/3 = | | 0.23 | 18 = 4.16 | 60/44 = | groups |
| | Zn | 40 = 0.76 | 0.031 | 8.13 | | | Mol | 36.4 | (OH)_{0.32} |
| | (int)/Ca- | | | Mol | | | fraction | Mol | Adduct and |
| | 10% | | | fraction | | | 4.16/17 = | fraction | trapped |
| | TP | | | 8.13/253 = | | | 0.24 | 36.4/60 = | internal |
| | (ext) | | | 0.032 | | | | 0.61 | water |
| | | | | | | | | | (H₂O)_{0.30} |
| 8755 | Zn- | Ca | Zn 2.0 | P 3.04 | 13.62 | 4.14 | 2.00 | 26.9 | Ca_{1.00}Zn_{0.04} |
| | ACC: | 30.7 | Mol | P → | | Mol | H₂O → | CO₂ → | (CO₃)_{0.80} |
| | 1% Ca- | Mol | fraction | (P₃O₁₀)/ | | fraction | 2OH | CO₃ | (P₃O₁₀)_{0.042} |
| | 6% | fraction | 2.0/ | 33.04 × | | 4.14/18 = | 2.0 × 34/ | 26.9 × | Overall OH |
| | w/w | 30.7/ | 65.4 = | 253/31/3 = | | 0.23 | 18 = 3.78 | 60/44 = | groups |
| | Zn | 40 = 0.77 | 0.031 | 8.27 | | | Mol | 36.7 | (OH)_{0.29} |
| | (ext)/Ca- | | | Mol | | | fraction | Mol | Adduct and |
| | 10% | | | fraction | | | 3.78/17 = | fraction | trapped |
| | TP | | | 8.27/253 = | | | 0.22 | 36.7/60 = | internal |
| | (int) | | | 0.033 | | | | 0.61 | water |
| 8758 | Zn- | Ca | Zn 2.00 | P 2.99 | 15.8 | 5.2 | 1.8 | 26.3 | Ca_{1.00}Zn_{0.041} |
| | ACC: | 29.6 | Mol | P → | | Mol | H₂O → | CO₂ → | (CO₃)_{0.81} |
| | 1% Ca- | Mol | fraction | (P₃O₁₀)/ | | fraction | 2OH | CO₃ | (P₃O₁₀)_{0.043} |
| | 6% | fraction | 2.00/ | 32.99 × | | 5.2/18 = | 1.8 × 34/ | 26.3 × | Overall OH |
| | w/w | 29.6/ | 65.4 = | 253/31/3= | | 0.29 | 18 = 3.4 | 60/44 = | groups |
| | Zn | 40 = 0.74 | 0.031 | 8.13 | | | Mol | 35.9 | (OH)_{0.27} |
| | (int/ext)/ | | | Mol | | | fraction | Mol | Adduct and |
| | Ca- | | | fraction | | | 3.4/17 = | fraction | trapped |
| | 10% | | | 8.13/253 = | | | 0.20 | 35.9/60 = | internal |
| | TP (int/ | | | 0.032 | | | | 0.60 | water |
| | ext) | | | | | | | | (H₂O)_{0.39} |
| 8799 | Zn- | Ca | Zn 1.42 | P 2.64 | 11.40 | 4.14 | 1.32 | 27.1 | Ca_{1.00}Zn_{0.029} |
| | ACC: | 30.7 | Mol | P → | | Mol | H₂O → | 27.1 × | (CO₃)_{0.81} |
| | 0.45% | Mol | fraction | (P₃O₁₀)/ | | fraction | 2OH | 60/44 = | (P₃O₁₀)_{0.036} |
| | Ca- | fraction | 1.42/ | 32.64 × | | 4.14/18 = | 1.32 × 34/ | 37.0 | Overall OH |
| | 5% | 30.7/ | 65.4 = | 253/31/3 = | | 0.23 | 18 = 2.49 | Mol | groups |
| | w/w | 40 = 0.77 | 0.022 | 7.18 | | | Mol | fraction | (OH)_{0.19} |
| | Zn/Ca- | | | Mol | | | fraction | 37.0/60 = | Adduct and |
| | 10% | | | fraction | | | 2.49/17 = | 0.62 | trapped |
| | TP | | | 7.18/253 = | | | 0.15 | | internal |
| | | | | 0.028 | | | | | water |
| | | | | | | | | | (H₂O)_{0.30} |
| 8800 | Zn- | Ca | Zn 3.06 | P 2.74 | 9.30 | 3.50 | 2.33 | 26.1 | Ca_{1.00}Zn_{0.047} |
| | ACC: | 30.0 | Mol | P → | | Mol | H₂O → | 26.1 × | (CO₃)_{0.59} |
| | 0.45% | Mol | fraction | (P₃O₁₀)/ | | fraction | 2OH | 60/44 = | (P₃O₁₀)_{0.029} |
| | Ca- | fraction | 3.06/ | 32.74 × | | 3.50/18 = | 2.33 × 34/ | 35.6 | Overall OH |
| | 10% | 30.0/40 | 65.4 = | 253/31/3 = | | 0.19 | 18 = 4.40 | Mol | groups |
| | w/w | 40 = 0.75 | 0.047 | 7.45 | | | Mol | fraction | (OH)_{0.19} |
| | Zn/Ca- | | | Mol | | | fraction | 35.6/60 = | Adduct and |
| | 10% | | | fraction | | | 4.40/17 = | 0.59 | trapped |
| | TP | | | 7.45/253 = | | | 0.26 | | internal |
| | | | | 0.029 | | | | | water |
| | | | | | | | | | (H₂O)_{0.30} |
| 8801 | Zn- | Ca | Zn 3.57 | P 0 | 8.65 | 3.38 | 1.09 | 33.6 | Ca_{1.00}Zn_{0.068} |
| | ACC: | 31.95 | Mol | | | Mol | H₂O → | 33.6 × | (CO₃)_{0.76} |
| | 0.45% | Mol | fraction | | | fraction | 2OH | 60/44 = | Overall OH |
| | Ca- | fraction | 3.57/ | | | 3.38/18 = | 1.09 × 34/ | 45.8 | groups |
| | 10% | 32.0/ | 65.4 = | | | 0.19 | 18 = 2.06 | Mol | (OH)_{0.12} |
| | w/w | 40 = 0.80 | 0.055 | | | | Mol | fraction | Adduct and |
| | Zn/Ca | | | | | | fraction | 45.8/60 = | trapped |
| | 0% | | | | | | 2.06/17 = | 0.76 | internal |
| | TP | | | | | | 0.12 | | water |
| | | | | | | | | | (H₂O)_{0.19} |
| 9998795 | AMC: | N/A | Mg | P 2.88 | 16.5 | 3.1 | 2.8 | 30.0 | Mg_{1.00}(CO₃)_{0.67} |
| | 1% | | 24.7 | P → | | Mol | H₂O → | CO₂ → | (P₃O₁₀)_{0.030} |
| | Mg- | | Mol | (P₃O₁₀)/ | | fraction | 2OH | CO₃ | Overall OH |
| | 10% | | fraction | 32.88 | | 3.1/18 = | 2.8 × 34/ | 30.0 × | groups |
| | TP | | 24.7/ | 253/31/3 = | | 0.17 | 18 = 5.3 | 60/44 = | (OH)_{0.31} |
| | | | 24.3 = | 7.83 | | | Mol | 40.9 | Adduct and |
| | | | 1.02 | Mol | | | fraction | Mol | trapped |
| | | | | fraction | | | 5.3/17 = | fraction | internal |
| | | | | 7.83/253 = | | | 0.31 | 39.5/60 = | water |
| | | | | 0.031 | | | | 0.68 | (H₂O)_{0.17} |
| 8796 | Mg- | Ca | Mg | P 2.3 | 17 | 3.4 | 2.3 | 27.9 | Ca_{1.00}Mg_{0.027} |
| | ACC: | 30.9 | 0.52 | P → | | Mol | H₂O → | CO₂ → | (CO₃)_{0.82} |
| | 0.45% | Mol | Mol | (P₃O₁₀)/ | | fraction | 2OH | CO₃ | (P₃O₁₀)_{0.032} |
| | Ca- | fraction | fraction | 32.3 × 253/ | | 3.4/18 = | 2.3 × 34/ | 27.9 × | Overall OH |
| | 5% | 30.9/ | 0.52/ | 31/3 = 6.26 | | 0.19 | 18 = 4.3 | 60/44 = | groups |
| | w/w | 40 = 0.77 | 24.3 = | Mol | | | Mol | 38.0 | (OH)_{0.33} |
| | Mg/Ca- | | 0.021 | fraction | | | fraction | Mol | Adduct and |
| | 10% | | | 6.26/253 = | | | 4.3/17 = | fraction | trapped |
| | TP | | | 0.025 | | | 0.26 | 38.0/60 = | internal |
| | | | | | | | | 0.63 | water |
| | | | | | | | | | (H₂O)_{0.25} |
| 8797 | Mg- | Ca | Mg | P 2.38 | 13.4 | 3.2 | 2.1 | 26.4 | Ca_{1.00}Mg_{0.064} |
| | ACC: | 29.9 | 1.18 | P → | | Mol | H₂O → | CO₂ → | (CO₃)_{0.80} |
| | 0.45% | Mol | Mol | (P₃O₁₀)/ | | fraction | 2OH | CO₃ | (P₃O₁₀)_{0.035} |
| | Ca- | fraction | fraction | 32.38 × | | 3.2/18 = | 2.1 × 34/ | 26.4 × | Overall OH |
| | 10% | 29.9/ | 1.18/ | 253/31/3 = | | 0.18 | 18 = 4.0 | 60/44 = | groups |
| | w/w | 40 = 0.75 | 24.3 = | 6.47 | | | Mol | 36.0 | (OH)_{0.31} |
| | Mg/Ca- | | 0.048 | Mol | | | fraction | Mol | Adduct and |
| | 10% | | | fraction | | | 4.0/17 = | fraction | trapped |
| | TP | | | 6.47/253 = | | | 0.24 | 36.0/60 = | internal |
| | | | | 0.026 | | | | 0.60 | water |
| | | | | | | | | | (H₂O)_{0.24} |
| 8798 | Mg- | Ca | Mg | P 0% | 7.83 | 3.71 | 0.5 | 35.5 | Ca_{1.00}Mg_{0.043} |
| | ACC: | 33.1 | 0.87 | | | Mol | H₂O → | 35.5 × | (CO₃)_{0.98} |
| | 0.45% | Mol | Mol | | | fraction | 2OH | 60/44 = | Overall OH |
| | Ca- | fraction | fraction | | | 3.71/18 = | 0.5 × 34/ | 48.4 | groups |
| | 10% | 33.1/ | 0.87/ | | | 0.21 | 18 = 0.94 | Mol | (OH)_{0.068} |
| | w/w | 40 = 0.83 | 24.3 = | | | | Mol | fraction | Adduct and |
| | Mg/Ca- | | 0.036 | | | | fraction | 48.4/60 = | trapped |
| | 0% TP | | | | | | 0.94/17 = | 0.81 | internal |
| | | | | | | | 0.056 | | water |
| | | | | | | | | | (H₂O)_{0.25} |
| | | | | | | | | | Crystallized |

Based on experimental data the inventors postulated that the particle further comprises metal oxide(s), such as in an amount ranging between 1 and 30 mol %, relative to the first metal content of the particle. Without being bound to any particular theory, it is resumed that at least some of metal hydroxides (Table 2) may be in a form of metal oxides, or in a form of mixed metal oxide/metal hydroxides.

### Example 3

### Stability of Suspensions of Stabilized Amorphous Metal Carbonates as-Prepared in their Reaction Suspension

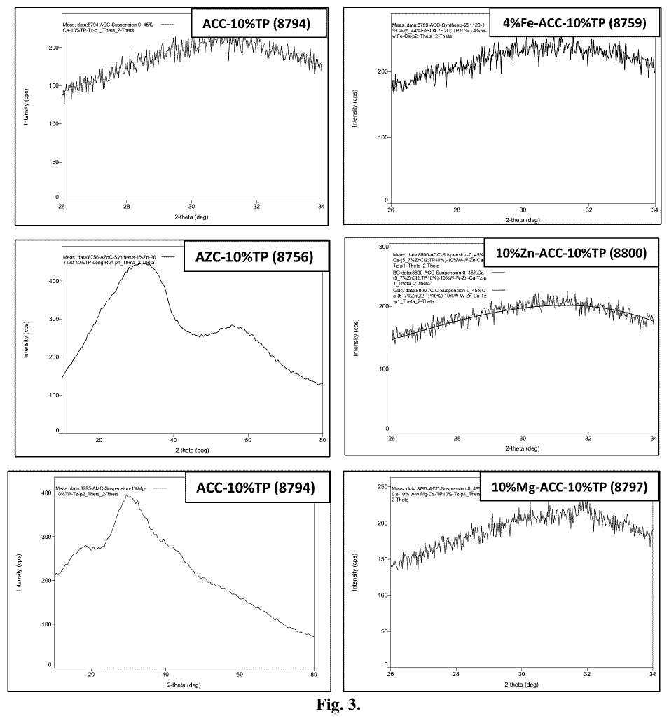

The stability of the amorphous metal carbonates in water-based liquids is very limited. It is critical to extend the duration of the amorphous metal carbonate stabilities in suspensions. Nevertheless, the shorter periods of suspension stabilities, in comparison to the long-term stabilities obtained with dry powders, provide a good method for rapidly screening the materials' stability. Table 3 documents a series of stability tests of suspensions of various amorphous metal carbonates, as prepared in their reaction solutions. Typical XRDs of amorphous metal carbonates are presented in FIG. 3.

amorphous metal carbonates have been synthesized as disclosed hereinabove. According to a general non-limiting procedure, the synthesis has been performed by adding 1 equivalent of sodium carbonate into a solution containing a total of one mol equivalent of a metal chloride and/or metal sulphate. A solution of sodium polytriphosphate as a stabilizer is added in most cases and mainly in a mode where half of the stabilizer is added with the sodium carbonate and half is added shortly after the precipitation.

In some cases, zinc chloride solution was added to a mostly ACC synthesis (a) together with the calcium chloride (int), after the ACC precipitation (ext.) or (c) in a combined mode (int/ext). In some cases a stabilizer was not added. The % number before the sign of the metal indicates the overall wt % of this metal in the suspension.

TABLE 3
Stability Studies of suspensions as, monitored by XRD analysis.

| | | Days in | |
|---|---|---|---|
| Lab Nb. | Sample Name | suspension | % ACC |
| 8794 | ACC-0.45% Ca-10% TP | 0 | 100 |
| 8794 | ACC-0.45% Ca-10% TP | 7 | 95 |
| 8794 | ACC-0.45% Ca-10% TP | 13 | 88 |
| 8794 | ACC-0.45% Ca-10% TP | 19 | 83 |
| 8794 | ACC-0.45% Ca-10% TP | 25 | 84 |
| 8794 | ACC-0.45% Ca-10% TP | 33 | 80 |
| 8794 | ACC-0.45% Ca-10% TP | 42 | 85 |
| 8795 | AMC-1% Mg-10% TP | 0 | 100% AMC |
| 8795 | AMC-1% Mg-10% TP | 1 | Mixture of |
| | | | Nesquehonite + AMC |
| 8795 | AMC-1% Mg-10% TP | 2 | Mixture of |
| | | | Nesquehonite + AMC |
| 8795 | AMC-1% Mg-10% TP | 13 | 100% Nesquehonite |
| 8796 | ACC-0.45% Ca-10% TP-5% w/w Mg/Ca | 0 | 100 |
| 8796 | ACC-0.45% Ca-10% TP-5% w/w Mg/Ca | 1 | 100 |
| 8796 | ACC-0.45% Ca-10% TP-5% w/w Mg/Ca | 2 | 99 |
| 8796 | ACC-0.45% Ca-10% TP-5% w/w Mg/Ca | 7 | 78 |
| 8796 | ACC-0.45% Ca-10% TP-5% w/w Mg/Ca | 13 | 76 |
| 8797 | ACC-0.45% Ca-10% TP-10% w/w Mg/Ca | 0 | 100 |
| 8797 | ACC-0.45% Ca-10% TP-10% w/w Mg/Ca | 6 | 98 |
| 8797 | ACC-0.45% Ca-10% TP-10% w/w Mg/Ca | 12 | 83 |
| 8797 | ACC-0.45% Ca-10% TP-10% w/w Mg/Ca | 18 | 78 |
| 8797 | ACC-0.45% Ca-10% TP-10% w/w Mg/Ca | 24 | 73 |
| 8797 | ACC-0.45% Ca-10% TP-10% w/w Mg/Ca | 32 | 72 |
| 8798 | ACC-0.45% Ca-0% TP-10% w/w Mg/Ca | 0 | 100 |
| 8798 | ACC-0.45% Ca-0% TP-10% w/w Mg/Ca | 1 | 20% ACC + 80% |
| | | | CCC |
| 8799 | ACC-0.45% Ca-10% TP-5% w/w Zn/Ca | 0 | 100 |
| 8799 | ACC-0.45% Ca-10% TP-5% w/w Zn/Ca | 1 | 100 |
| 8799 | ACC-0.45% Ca-10% TP-5% w/w Zn/Ca | 6 | 99 |
| 8799 | ACC-0.45% Ca-10% TP-5% w/w Zn/Ca | 12 | 97 |
| 8799 | ACC-0.45% Ca-10% TP-5% w/w Zn/Ca | 18 | 91 |
| 8799 | ACC-0.45% Ca-10% TP-5% w/w Zn/Ca | 24 | 91 |
| 8799 | ACC-0.45% Ca-10% TP-5% w/w Zn/Ca | 32 | 94 |
| 8799 | ACC-0.45% Ca-10% TP-5% w/w Zn/Ca | 41 | 91 |

Legend: ACC = amorphous calcium carbonate; AMC = amorphous magnesium, carbonate; AZC = amorphous zinc carbonate; TP = polytriphosphate; int = incorporation of the metal or the stabilizer during the initial synthesis step; ext = adding the second or more metal or the stabilizer after the initial precipitation of the ACC. The X % before the sign of the metal indicates its total wt % in the suspension. Wt % or mol % of a metal is given in the format of X % w/w or X % mol/mol of one metal compared to the other.

Furthermore, the inventors successfully confirmed suspension stability (for at least 7 days) of various amorphous metal carbonate particles of the invention, such as Ca/Mg (about 5-10% w/w Mg) with about 10% stabilizer; Ca/Mg (about 10-30 mol % Mg) with or without 4 mol % stabilizer; Ca/Zn (about 5-10% w/w Zn) with or without stabilizer; Ca/Zn (about 10-30 mol % Zn) with 4 mol % stabilizer; AZC (with about 10% stabilizer); Ca/Fe(II) (about 5-10% w/w Fe) with about 10% stabilizer; Ca/Fe(II) (about 30 mol % w/w Fe) with about 4 mol % stabilizer; Ca/Cr(II) (about 5-10% w/w Cr) with about 10% stabilizer; Ca/Mn (about 5-10% w/w Mn) with about 10% stabilizer.

Furthermore, the inventors successfully confirmed suspension stability (for at least 7 days) of a triple divalent metal particle: Ca/Mg/Zn amorphous carbonate comprising 85% Ca: 10% Mg: 5% Zn, and 10% TP.

Additional single divalent and mixed/mixed stabilized particles of the invention have been synthesized as described hereinbelow. Subsequently, the aqueous suspension stability of the synthesized particles have been examined. The results of the long-term stability studies are summarized in Table 3A.

General Method for Preparation of Stabilized Mixed Particles Comprising Ca as the First Metal and Further Comprising a Metal (M) (M=Zn, Fe, Cr, Mg, Mn) and a Stabilizer (Sodium Tripolyphosphate (STPP or TPP) or Phosphoserine (PS)

In a typical procedure, a metal salt or mixed metal salts (preferably, chloride, nitrate or sulphate) solution (80 ml water with 0.05 mol of the metal ions) is mixed with a sodium carbonate solution (80 ml of water, 5.3 g of sodium carbonate, 0.05 mol).

In some cases, a sodium tripolyphosphate (STPP) solution (typically 0.74 g of Sodium tripolyphosphate; typically added as 10% w/w of the weight metal reagent or mol % thereof), is added as a stabilizer during, after, or both during and after the mixing the metal solutions with the carbonate solution. In other cases, phosphoserine (PS), is added in a solution form (typically 0.925 g of Phosphoserine, 0.005 mol PS, 10% mol/mol of combined metals).

In many cases a metal (M) was added at different concentrations at the following ratios M/Ca (for example Zn in Ca, Fe in Ca, Mg in Ca or Cr in Ca) at various wt % or mol % of the primary metal mol at the following range of concentrations: 1 to 30% mol/mol of the primary metal (e.g., calcium).

Mixed stabilizers were sometimes added as either a mixture of stabilizers 1, 2 or more, of which their final volume is divided into two equal parts, the first part was added consecutively to one of the reaction solutions before mixing the metal and the carbonate reagents. Then the second part was added immediately after the addition of the sodium carbonate solution to the metal salt solution (including the first part of the mixed stabilizers). This process is defined as “4Pack” reaction or Internal/External. In a typical reaction, the final volume of the mixed stabilizer solution should reach a volume of 40 ml, thus that the final reaction volume does not exceed 200 ml in purpose to attain around 1 to 2% w/v metal concentration.

Another method comprises adding the stabilizer(s) to one of the reactant solutions before the formation of the metal carbonate. In this case the stabilizer is defined as “Internal”. Alternatively, the stabilizer solution can be added after the initial reaction and the precipitation of the vast majority of the metal carbonate. Then the stabilizer is defined as “External”.

The total of the metal ions used as reagents is in an equimolar quantity compared to the carbonate ions. The final product of amorphous metal carbonate was vacuum filtered using a Buchner, washed with ethanol in abundant quantities, and dried, by oven with a ventilation valve at 100° C. for not more than 30 minutes. In an equivalent industrial process at large quantities can be dried by a variety of industrial dryers and ovens, without the need to wash with ethanol for safety, environmental and cost purposes. The powder obtained above was analyzed using X-Ray diffraction, ICP, TGA, DSC, SEM, BET and in several cases by TEM.

TABLE 3A
stability of suspensions of various amorphous
metal carbonates, stabilized by phosphoserine

| Batch | | % Amorphous |
|---|---|---|
| No. | Sample Name | Phase |
| 58 | ACC-Suspension-0.45% Ca-5% mol PS-30% mol Zn-4PACKS-Tz | 100 |
| 58 | ACC-Suspension-0.45% Ca-5% mol PS-30% mol Zn-4PACKS-DAY 1 | 100 |
| 58 | ACC-Suspension-0.45% Ca-5% mol PS-30% mol Zn-4PACKS-DAY 2 | 100 |
| 58 | ACC-Suspension-0.45% Ca-5% mol PS-30% mol Zn-4PACKS-DAY 8 | 100 |
| 58 | ACC-Suspension-0.45% Ca-5% mol PS-30% mol Zn-4PACKS-DAY 15 | 100 |
| 58 | ACC-Suspension-0.45% Ca-5% mol PS-30% mol Zn-4PACKS-DAY 28 | 100 |
| 59 | AZC-Suspension-0.45% Zn-5% mol PS-4PACKS-Tz | 100 |
| 59 | AZC-Suspension-0.45% Zn-5% mol PS-4PACKS-DAY 1 | 100 |
| 59 | AZC-Suspension-0.45% Zn-5% mol PS-4PACKS-DAY 2 | 100 |
| 59 | AZC-Suspension-0.45% Zn-5% mol PS-4PACKS-DAY 8 | 100 |
| 59 | AZC-Suspension-0.45% Zn-5% mol PS-4PACKS-DAY 15 | 100 |
| 59 | AZC-Suspension-0.45% Zn-5% mol PS-4PACKS-DAY 28 | AZC + ZC |
| 60 | AMC-Suspension-0.45% Mg-5% mol PS-4PACKS-Tz | 100 |
| 60 | AMC-Suspension-0.45% Mg-5% mol PS-4PACKS-DAY 1 | 100 |
| 60 | AMC-Suspension-0.45% Mg-5% mol PS-4PACKS-DAY 8 | AMC + |
| | | Nesquehonite |

Furthermore, suspension stability of Ca/Mg particles with 5% mol Mg (relative to Ca) and stabilized with 30 mol % PS has been examined. The resulting suspensions were substantially stable for at least about 24 h.

Table 3A summarizes the stability of selected amorphous metal carbonates, stabilized with 5 mol % of phosphoserine, compared to the amount of calcium and carbonate reagents added to a typical “4Pack” synthesis, in which the stabilizer is divided to 2 equal portions (2.5 mol % each). Then one portion is added to either the calcium chloride solution or the sodium hydroxide solution before the reaction begins. Half of the stabilizer (2.5 mol %) was added to the sodium carbonate solution prior to the mixing with the calcium chloride and half was added after the completion of the calcium and carbonate reagents, within 2 minutes.

Nomenclature: “ACC-Suspension-0.45% Ca-5% mol PS-30% mol Mg-4PACKS-Tz” means: ACC suspension containing 0.45% calcium, stabilized by 5 mol % of phosphoserine, in which 30 mol % of compared to the calcium is present. The stabilizer was added in a 4pack process (as described above). Tz=time zero; Dayl, 2, etc., means the numbers of days of the stored suspension before the XRD analysis. ACC=amorphous zinc carbonate; AZC=amorphous zinc carbonate; AMC=amorphous magnesium carbonate.

As represented by Table 3A phosphoserine and polytriphosphate are both capable of stabilizing aqueous suspensions of the particles of the invention. Especially in combination with Zn, PS results in very stable amorphous carbonate particles. The suspension stability was analyzed by filtering and drying powder from the suspension and measuring the amorphous status by XRD.

Without being bound to any particular theory, based on the experimental data, the inventors postulated that amorphous aqueous formulations without an additional stabilizing compound have only limited stability when stored in a form of an aqueous suspension. It has been observed, that aqueous suspensions of amorphous metal carbonates are in most cases stable for a period of about 24 h.

Without being bound to any particular theory, based on the experimental data, the inventors postulated that about 10% wt of the stabilizer relative to the weight of the metal reagent (or at least 1 mol % relative to the first metal) is sufficient to stabilize the amorphous phase of the tested metal carbonates in an aqueous suspension. Most of the synthesized amorphous metal carbonates exhibited substantial suspension stability, retaining at least 95% of the metal carbonate in an amorphous state for a time period greater than 50 days.

Amorphous phase stability of mixed (and/or mixed stabilized) particles having Ca as the first metal and further comprising an additional divalent metal reveal similar morphologies provided by the basic synthesis approach. Compositions containing between 1 to 30 mol % of the second divalent metal relative to Ca were efficiently synthesized. It is further postulated, that less than 0.5 mol %, or less than 1 mol % of the second divalent metal relative to the first divalent metal would result in unstable particle or wouldn't be applicable in an industrial process (e.g., resulting in crystallization of the metal carbonate).

### Example 4

### Stability of as-Prepared Suspensions of Stabilized Amorphous Zn-ACC with Various Fractions of Zn and Triphosphate

A series of Zn-ACC-TP compositions were synthesized by incorporating the Zn reagent and/or the triphosphate either during the initial precipitation reaction or after it occurs. The stability results are presented in Table 4. The reaction suspensions demonstrate adequate stability in all cases, in-spite of the lower ratio of TP to Ca, than the usual 10% TP, used in the previous examples. Hence, it is concluded that the Zn demonstrates a stabilizing effect by itself. Indeed, higher stability is obtained when the Zn fraction is doubled.

TABLE 4
Stability of suspensions comprising ZN-ACC stabilized by TP

| | | % Amorphous |
|---|---|---|
| Batch No. | | Phase |
| 7906 | 0.45% Ca-10% TP-0% w/w Zn/Ca | 100 |
| | Day 2 | |
| 7906 | Day 5 | 95 |
| 7906 | Day 7 | 91 |
| 7906 | Day 9 | 88 |
| 7906 | Day 12 | 80 |
| 7906 | Day 15 | 79 |
| 7907 | 0.45% Ca-(2% ZnCl₂)/(6% TP)- | 100 |
| | 3.52% w/w Zn/Ca Day 2 | |
| 7907 | Day 5 | 97 |
| 7907 | Day 7 | 94 |
| 7907 | Day 9 | 91 |
| 7907 | Day 12 | 94 |
| 7907 | Day 15 | 84 |
| 7908 | 0.45% Ca-(6% TP)/(2% ZnCl₂)- | 100 |
| | 3.52% w/w Zn/Ca Day2 | |
| 7908 | Day 5 | 97 |
| 7908 | Day 7 | 93 |
| 7908 | Day 9 | 89 |
| 7908 | Day 12 | 88 |
| 7908 | Day 15 | 79 |
| 7909 | 0.45% Ca-(4% ZnCl₂, 6% TP)- | 100 |
| | 7.04% w/w Zn/Ca Day2 | |
| 7909 | Day 5 | 100 |
| 7909 | Day 7 | 98 |
| 7909 | Day 9 | 97 |
| 7909 | Day 12 | 97 |
| 7909 | Day 15 | 96 |

Accordingly, based on the experimental data present in Table 4, it is postulated that the minimum weight ratio of the stabilizer to the first metal within the mixed stabilized particle of the invention is about 6%, or less.

### Example 5

### Effects of Various ACC and CCC Suspensions on Acidified Serum in Medium

The inventors evaluated the ability of ACC, AMC, AZC and various M-ACCs with and without stabilizers to formulated with different stabilizers, to affect the pH of medium supplemented with 10% (v/v) serum. The example demonstrates the rapid pH response when the serum is acidified at levels found around tumors and inflammations. It also demonstrates that the level of the final pH control is feasible by the compositions of the formulae, where the type and amount of the metals and stabilizers affect their pH modulation capability. The example also records how the various formulations (a) maintain their stability in fresh as made suspensions, (b) affect the initial pH of the suspensions, and (c) maintain the stability of the amorphous phases of the various formulations.

To 18 ml of medium DMEM/F12 (Biological Industries, Beit Haemek, Israel), 2 ml of fetal bovine serum (FBS, Biological Industries, Beit Haemek, Israel) was added. The solution was placed inside a sterile tissue-sample cup, and a hole was made at the top of the cup to which a pH probe was inserted. The cup was placed on a magnetic stirrer (JB-10 stirrer, Inesa, China) and the solution was constantly stirred during the measurement. A pH meter (MesuLab, PXSJ-216F ion meter, MRC, Israel) was connected to a PC the data logging of the pH measurements was performed using the software REXDC2.0.

After setting the system and starting the pH measurement and data logging, an amount of 20.5 μl of lactic acid was added to the solution in order to reduce the pH to a slightly acidic pH. After a few seconds and the stabilization of the pH, an amount of 3 ml of freshly prepared ACC suspension was added to the solution.

Table 5 below summarizes the immediate and long-time changes of the pH curves as a function of the suspension composition. The table also indicates the amorphous phase stability of the various formulations as a function of time.

TABLE 5
Time dependent pH of the serum upon addition of various particles of the
invention

| | | | Initial | | | XRD |
|---|---|---|---|---|---|---|
| | | | pH of | pH 20 | pH 400 | after |
| | | PH | acidified | sec after | sec after | Synthesis |
| | | Carbonate | Serum | carbonate | carbonate | & |
| Exp# | Synthesis Name* | Suspension | solution | addition | addition | Drying |
| 8849 | ACC-PS-CA: | | 7.04 | 8.15 | 8.28 | |
| | 1.57% Ca-1% PS- | | | | | |
| | 5% CA | | | | | |
| 8751 | Fe-ACC: 1% Ca-2% | 9.03 | | | | 100 |
| | w/w Fe/Ca-10% TP | | | | | |
| 8752 | Zn-ACC: 1% Ca-2% | 8.96 | | | | 98 |
| | w/w Zn(int)/Ca- | | | | | |
| | 10% TP(ext) | | | | | |
| 8754 | Zn-ACC: 1% Ca-6% | 8.80 | | | | 100 |
| | w/w Zn(int)/Ca- | | | | | |
| | 10% TP(ext) | | | | | |
| 8755 | Zn-ACC: 1% Ca-6% | 8.62 | | | | 99 |
| | w/w Zn(ext)/Ca- | | | | | |
| | 10% TP(int) | | | | | |
| 8756 | AZC: 1% Zn- | 6.63 | 6.31 | 6.92 | 7.12 | 100 |
| (8851) | 10% TP | | | | | |
| 8757 | Zn-ACC: 1% Ca-2% | | | | | 100 |
| | w/w Zn(ext)/Ca- | | | | | |
| | 10% TP(int) | | | | | |
| 8758 | Zn-ACC: 1% Ca-6% | 8.70 | | | | 100 |
| | w/w Zn(int/ext)/Ca- | | | | | |
| | 10% TP(int/ext) | | | | | |
| 8759 | Fe-ACC: 1% Ca-4% | 8.78 | | | | 100 |
| | w/w Fe/Ca-10% TP | | | | | |
| 8794 | ACC-TP: 0.45% Ca- | 10.25 | 7.06 | 7.98 | 8.20 | 100 |
| (8848) | 10% TP | | | | | |
| 8795 | AMC: 1% Mg- | 9.22 | | | | 100 |
| | 10% TP | | | | | |
| 8796 | Mg-ACC: 0.45% Ca- | 9.95 | 7.04 | 8.12 | 8.15 | 100 |
| (8850) | 5% w/w Mg/Ca-10% | | | | | |
| | TP | | | | | |
| 8797 | Mg-ACC: 0.45% Ca- | 9.54 | | | | 100 |
| | 10% w/w Mg/Ca- | | | | | |
| | 10% TP | | | | | |
| 8798 | Mg-ACC: 0.45% Ca- | 9.50 | | | | 100 |
| | 10% w/w Mg/Ca- | | | | | |
| | 0% TP | | | | | |
| 8799 | Zn-ACC: 0.45% Ca- | 9.24 | 6.21 | 7.55 | 7.64 | 100 |
| (8852) | 5% w/w Zn/Ca- | | | | | |
| | 10% TP | | | | | |
| 8800 | Zn-ACC: 0.45% Ca- | 9.08 | | | | 100 |
| | 10% w/w Zn/Ca- | | | | | |
| | 10% TP | | | | | |
| 8801 | Zn-ACC: 0.45% Ca- | 8.70 | | | | 100 |
| | 10% w/w Zn/Ca- | | | | | |
| | 0% TP | | | | | |
| 8803 | Fe-ACC: 0.45% Ca- | 9.20 | 6.55 | 7.88 | 7.99 | 100 |
| (8854) | 5% w/w Fe/Ca- | | | | | |
| | 10% TP | | | | | |
| 8804 | Fe-ACC: 0.45% Ca- | 8.80 | 6.4 | 7.66 | 7.71 | 100 |
| (8855) | 10% w/w Fe/Ca- | | | | | |
| | 10% TP | | | | | |
| 8805 | Fe-ACC: 0.45% Ca- | 8.49 | | | | 100 |
| | 10% w/w Fe/Ca- | | | | | |
| | 0% TP | | | | | |

*Nomenclature of experiments: ACC = amorphous calcium carbonate, AMC = amorphous magnesium carbonate, AZC = amorphous zinc carbonate; stabilizers: TP = triphosphate, PS = phosphoserine, CA = citric acid; int = addition of reagent during the initial mixing of the calcium and carbonate solutions, ext = addition of the reagent after the initial ACC precipitation.
The way to decode the synthesis name: “1% Ca-6% w/w Zn(int)/Ca-10% TP(ext)” means 1% total calcium in the suspension; the metal (zinc) fraction compared to calcium by weight is 6%; the stabilizer fraction (TP) compared to calcium by weight is 10%; the Zn solution was added in the initial reaction and the TP was added after the precipitation. If int. or ext. are not mentioned, then the metal solution was added to the calcium solution first and the stabilizer was added half during the formation of the carbonate and half after the initial precipitation.

### Example 6

The inventors demonstrated that the stabilizers are incorporated into the bulk of the ACC particle.

In a typical synthesis of ACC, wherein polytriphosphate (“TP” with a chain of only 3 phosphates or “STPP”, standing for sodium tripolyphosphate) is added during and after the formation of the ACC, the amount of the added TP was varied: 5, 10 and 15 mol % compared to the amount of the calcium reagent that was added to the synthesis. These amounts of TP were added as follow: (a) one half (0.50) was introduced to the reaction solution simultaneously with the carbonate solution; (b) one half (0.50) was added immediately after the completion of the very rapid formation of the suspension. Based on the low solubility of ACC, it is assumed that the ACC formation is completed within seconds, since the suspension and its color intensity is achieved within a few seconds of mixing the calcium and the carbonate clear solution.

Powders obtained in the above suspensions were thoroughly washed and dried and checked for being 100% amorphous. By the washing procedure any traces of the reagents including any STPP that is not interacting chemically with the ACC.

Sample were submitted for ICP (for determining Ca, P, Na and Cl quantitative levels), TGA (for quantitative determination of the carbonate by the measurement of released of CO2), solid state NMR (see FIG. 4), SEM (see FIG. 1) and TEM.

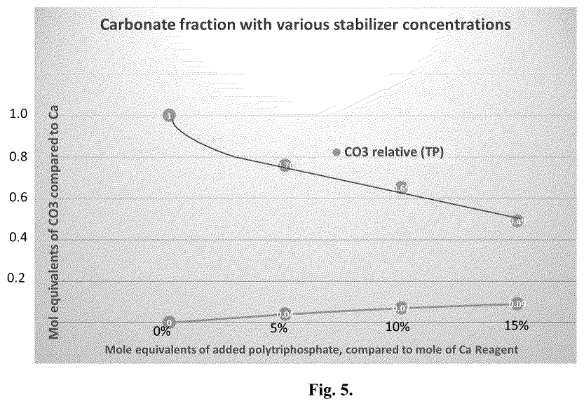

The results of the combined analysis of ICP and TGA have been summarized in FIG. 5. Without being limited to any particular theory, and solely based on the graph represented in FIG. 5, the inventors concluded that:
(1) the incorporation of TP is quantitative and almost linear with slight rate reduction as its concentration increases. This is a typical situation in cases where there is competition between reagents (in this case the carbonate ions) on reacting with another reagent.
(2) although the incorporation of the TP is quantitative, it is not sum up to the amounts of STPP, introduced into the reaction in both steps. It is assumed that most of the residual soluble TP which is not interacting with the ACC is coming from the second step and is washed away during the isolation of the ACC as powder.
(3) Simultaneously, the amount of carbonate is reduced quantitively, also, in a linear relation with the STPP concentration. However, the reduction of carbonate equivalents is about 5 to 6 times over the equivalents of incorporated TP. It means that each phosphate unit of the TP molecule occupies 1.5 to 2 calcium ions, on average, out of capacity of 2.5 Ca:TP capability. It is anticipated that TP adsorbed at the surface, will have lesser binding to calcium ions.
(4) At low concentration of STPP, the reduction of the carbonate is slightly curved, suggesting that at the beginning the chemically bonded adsorption of the TP to the ACC particle surface, is more dominant until saturation of the surface with a monolayer of TP is achieved.

The above observations give a strong evidence for the incorporation of the stabilizer inside the molecular network of the amorphous metal carbonate particle of the invention. These conclusions are also supported with the following observations: (i) both the SEM analyses and TEM analyses (FIGS. 1 and 5) reveal similar homogeneous morphologies of spherical primary particles that are agglomerated; (ii) mapping performed with TEM, reveals even distribution of P within the particles like the distribution of the Ca. There is no higher intensity of P observed at the surface of the particles, indicating that TP is substantially uniformly distributed within the entire particle and at the particle's surface.

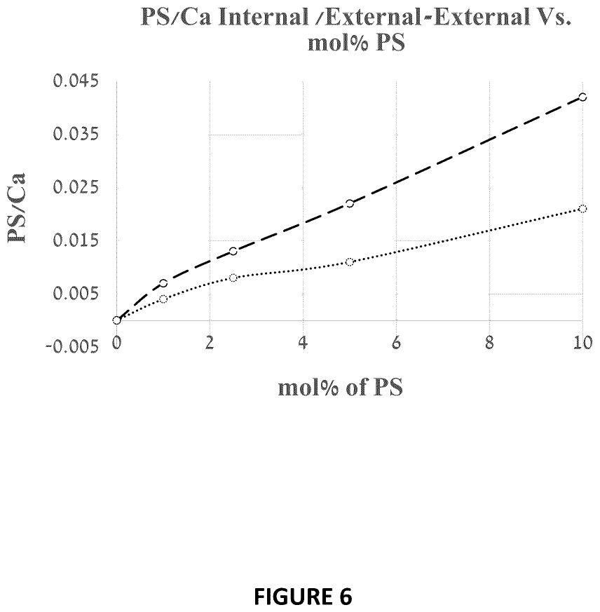

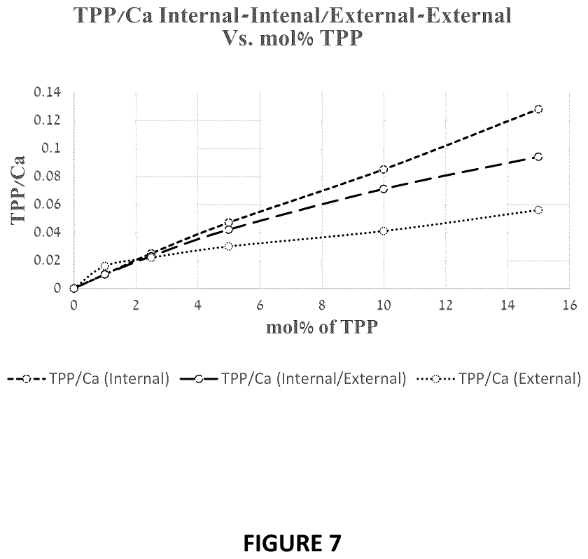

FIGS. 6 and 7 illustrate the levels of incorporating phosphoserine and tripolyphosphate stabilizers into the entire particle of the invention and/or at its surface as a function of (a) type of stabilizer, (b) amount of added stabilizer, and (c) the mode the stabilizer is added (as a part of the synthesis, after the synthesis or both). In parallel, based on the experimental findings, the inventors postulated that there is a reduction of the CO3 content. Although both PS and TPP show slightly different behavior, it is clear that the stabilizer can saturate the surface area relatively fast and is incorporated into the structure by replacing carbonate anions. The more the stabilizer was added as a part of the synthesis the more it was incorporated into the particles. Without being bound to any particular theory, it is postulated that TPP is overall incorporated in significantly higher yields compared to PS and also displaces much more carbonate units.

Furthermore, it has been observed (as represented by FIGS. 6 and 7) that the location of the stabilizer is predefined inter alia by the ratio of stabilizer to the amorphous metal carbonate. In particular, it is postulated that by implementing more than 3 mol % of the stabilizer during the synthesis of the particle of the invention, the stabilizer is predominantly located in the interior of the particle. It is further postulated that the location of the stabilizer is predetermined inter alia by (i) the manufacturing process (e.g. whether the stabilizer has been added concomitantly or subsequently to the metal carbonate particle formation); (ii) the concertation of the stabilizer in the manufacturing process and (iii) by the chemical composition of the stabilizer.

### Example 7

### BET Measurements

Numerous amorphous metal carbonate powders have been analyzed to determine porosity and BET surface area thereof. Table 6 summarizes the surface areas, measured by BET.

TABLE 6
BET surface are of the exemplary powderous compositions of the invention

| | Surface Area |
|---|---|
| Sample | (m²/g) |
| ACC-6 wt % STTP | 34.0, 53.3, 69.8 |
| | (different batches) |
| ACC-0.45% Ca-30% mol/mol Mg/Ca-4% mol/mol STPP/Ca | 45.5 |
| ACC- 0.45% Ca-30% mol/mol Mg/Ca-0% mol/mol STPP/Ca | 42.2 |
| ACC- 0.45% Ca-30% mol/mol Zn/Ca-4% mol/mol STPP/Ca | 48.3 |
| ACC- 0.45% Ca-30% mol/mol Zn/Ca-0% mol/mol STPP/Ca | 45.3 |
| ACC- 0.45% Ca-30% mol/mol Fe/Ca-4% mol/mol STPP/Ca | 105.8 |
| ACC- 0.45% Ca-30% mol/mol Fe/Ca-0% mol/mol STPP/Ca | 74.4 |
| 4892-AMC-Synthesis-280519-1.13% Mg-0% TP | 34.0 |
| 4893-AMC-Synthesis-280519-1.13% Mg-6.4% TP | 53.4 |
| 4894-AMC-Synthesis-280519-1.13% Mg-15.1% TP | 69.8 |
| 9223-ACC-Suspension-070421-0.45% Ca-30% mol/mol | 45.5 |
| Mg/Ca-4% mol/mol STPP/Ca | |
| 9224-ACC-Suspension-070421-0.45% Ca-30% mol/mol | 42.2 |
| Mg/Ca-0% mol/mol STPP/Ca | |
| 9225-ACC-Suspension-070421-0.45% Ca-30% mol/mol | 48.3 |
| Zn/Ca-4% mol/mol STPP/Ca | |
| 9226-ACC-Suspension-070421-0.45% Ca-30% mol/mol | 45.3 |
| Zn/Ca-0% mol/mol STPP/Ca | |
| 9227-ACC-Suspension-070421-0.45% Ca-30% mol/mol | 105.8 |
| Fe(II)/Ca-4% mol/mol STPP/Ca | |
| 9228-ACC-Suspension-070421-0.45% Ca-30% mol/mol | 74.4 |
| Fe(II)/Ca-0% mol/mol STPP/Ca | |
| 9509-ACC-Suspension-0.45% Ca-10% mol/mol Mg/Ca-2% | 34.7 |
| mol/mol STPP/Ca | |
| 9511-ACC-Suspension-0.45% Ca-10% mol/mol Zn/Ca-2% | 48.7 |
| mol/mol STPP/Ca | |
| 9512-ACC-Suspension-0.45% Ca-10% mol/mol Zn/Ca-0% | .378 |
| mol/mol STPP/Ca | |
| 9513-ACC-Suspension-0.45% Ca-10% mol/mol Fe(II)/Ca-2% | 62.1 |
| mol/mol STPP/Ca | |
| 9514-ACC-Suspension-0.45% Ca-10% mol/mol Fe(II)/Ca-0% | .383 |
| mol/mol STPP/Ca | |
| 9515-ACC-Suspension-0.45% Ca-20% mol/mol Mg/Ca-4% | 40.9 |
| mol/mol STPP/Ca | |
| 9516-ACC-Suspension-0.45% Ca-20% mol/mol Mg/Ca-0% | 34.5 |
| mol/mol STPP/Ca | |
| 9517-ACC-Suspension-0.45% Ca-20% mol/mol Zn/Ca-4% | .333 |
| mol/mol STPP/Ca | |
| 9518-ACC-Suspension-0.45% Ca-20% mol/mol Zn/Ca-0% | 38.1 |
| mol/mol STPP/Ca | |
| 9519-ACC-Suspension-0.45% Ca-20% mol/mol Fe(II)/Ca-4% | .791 |
| mol/mol STPP/Ca | |
| 9520-ACC-Suspension-0.45% Ca-20% mol/mol Fe(II)/Ca-0% | .447 |
| mol/mol STPP/Ca | |

The range of the surface area for all the various pure and mixed metal carbonates is in the range of 34 to 106 m²/g. Overall, the surface area is in the range of 30 to 70 m²/g for all tested various amorphous carbonate particles of the invention which do not contain Fe(II) (e.g. single divalent metal particle and mixed/mixed stabilized metal particles), indicating nanoparticles with average particle sizes in the range of 20 to 60 nm, as indeed determined by SEM and TEM analyses. Exemplary particles of the invention containing Fe(II) provide surface area of 70 to 105 m²/g, indicating nanoparticles with an average particle sizes below 20 nm.

BJH pore size and pore volume analysis of ACC 6 wt % STPP reveals that these nanoparticles have very low open porosity at the level of 0.06 to 0.10 cc/g with pore size diameter of around 1 to 2 nm.

### Example 8

### Solid State NMR and TEM Analysis of Amorphous Metal Carbonates

Solid State ¹³C NMR analyses of exemplary single 100% amorphous metal carbonate particles and mixed particles of the invention (a second metal within ACC), with polytriphosphate as a stabilizer or without a stabilizer were performed. The accumulated and overalled A single peak of carbonate is observed for all the materials as shown in FIG. 4. ACC and all the metals incorporated within ACC show a broad peak of carbonate with a maximum height in the range of 168.5+/−1.0 ppm. This is the same place the calcite's calcite carbonate peak appears. However, the latter one is much sharper than the amorphous carbonates.

Carbonate peaks of ACC stabilized by mono- and diphosphate (pyrophosphate) were at the same ppm range and width. It should be noted that carbonates of ACC, stabilized with phosphoserine also has a peak maximum at 168.6 ppm. Hence, the carbonate peak position is not significantly affected by the type of stabilizer and the presence of additional metals within the ACC matrix.

The carbonate peaks of additional exemplary single metal carbonate particles of the invention containing metals such as Mg and Zn, have also a single broad carbonate peak, which is slightly shifted. The peak maxima in the cases of amorphous Mg and Zn carbonates are at 166.1 and 165.3, respectively.

High Resolution transmission electron microscopy (HR-TEM) analyses of various synthesized metal carbonate were performed, including energy dispersive X-ray spectroscopy (EDS) mapping of Ca and P of the exemplary compositors of the invention.

Based on the TEM analyses the inventors concluded that some particles are characterized by minimal porosity, most likely close porosity, in the range of 2 to 10 nm. Such close porosity is expected to capture water beyond simple adsorption as indicated by TGA analyses of exemplary compositions of the invention.

Additionally, based on the TEM analyses the inventors concluded that exemplary particles of the invention are substantially devoid of porosity (such as amorphous magnesium carbonate (AMC), or amorphous zinc AZC stabilized by 10% tripolyphosphate). The EDS mapping of Ca (red) and P (blue) elements indicate even distribution of P throughout the particles. Such even distribution is another evidence for the homogeneous incorporation of the phosphate stabilizer within the internal molecular structure of the particle.

### Example 9

### The Influence of Stabilized Amorphous Carbonate Metals on Cathepsins Activity

### Materials and Method

### Culture Medium

All materials were purchased from Sartorius—Biological Industries, Beit Haemek, Israel, unless specified differently. The complete culture medium was composed of 90% Dulbecco's modified Eagle medium-nutrient mixture F-12 (DMEM-F12) calcium depleted, 10% (v/v) fetal bovine serum (FBS), 2 mM glutamine, Penicillin G Sodium Salt: 10,000 units/mL, Streptomycin Sulfate: 10 mg/mL (Pen/Strep). Calcium chloride in 1 mM final concentration was added to the media for bank proliferation or for other metal carbonate testes.

### Thawing and Culturing

All procedures were performed in a laminar flow hood, under aseptic conditions. One frozen ampule of A549 cancer cells was thawed in a 37° C. water bath and seeded into two T-25 flasks (Corning Inc, NY, USA) filled with 5 ml of complete medium and 1 mM calcium chloride. The next day the medium was replaced. Then, medium was replaced twice a week. After the cells reached confluency, the cells were sub-culture in a spilt ratio of 1:10 into new T-25 flasks. The cells were split into 9 groups.

Tested Groups: (1) Complete medium (calcium depleted), with addition of 1 mM calcium chloride (CaCl₂)) (Herein Bank); (2) Complete medium (calcium depleted) medium with 2 mM Ca⁺² and Carbonate originated from Crystalline Calcium Carbonate=CCC (Herein CCC Test Group). 40 μl suspension into 1 ml media; (3) Complete medium (calcium depleted) medium with addition of 1 mM calcium chloride (CaCl₂)), oriented from CaCl₂) and 1.2 mM Mg⁺² and Carbonate oriented from AMC (Herein AMC Test Group). 24 μlsuspension into 1 ml media; (4) Complete medium (calcium depleted) medium with addition of 1 mM calcium chloride (CaCl₂)), oriented from CaCl₂) and 10 μM Zn⁺² and Carbonate oriented from AZC (Herein AZC Test Group). 0.2 μlsuspension into 1 ml media; (5) Complete medium (calcium depleted) medium with addition of 1.8 mM calcium chloride (CaCl₂)), oriented from ACC and 0.2 mM Mg⁺² and Carbonate oriented from ACC-AMC (ACMC) (Herein ACMC Test Group). 40 μl suspension into 1 ml media; (6) Complete medium (calcium depleted) medium with addition of 1.99 mM calcium chloride (CaCl₂)), oriented from ACC and 10 μM Zn′ and Carbonate oriented from ACC-AZC (ACZC) (Herein ACZC Test Group). 40 μl suspension into 1 ml media; (7) Complete medium (calcium depleted) medium with addition of 1.79 mM calcium chloride (CaCl₂)), oriented from ACC and 0.2 mM Mg+2 and 10 μM Zn⁺² and Carbonate oriented from ACC-AMC-AZC (ACMZC) (Herein ACMZC Test Group). 40 μl suspension into 1 ml media.

Each group included two replicates (duplicate). The culture continues for 3-5 passages and then part of the cells (from each group) were spanned to create a frozen bank and the other cells were taken for the Cathepsin B activity test. Media were replaced twice a week with a fresh medium, containing the treatment. All treatments solutions: ACC, CCC, AMC, AZC, ACMC, ACZC, ACMZC, MgCl₂, ZnCl₂ and CaCl₂) were prepared fresh once a week. Once the cells reached confluency, cells were subcultured into new flasks in a dilution ratio 1:10, twice a week in a fresh medium, containing the same individual treatment. All treatments were subcultured together.

After 3-5 passages cells were lysed in Cathepsin B Lysis buffer, and protein amount/concentration for each sample was measured and determined with Bradford reagent in spectrophotometer at 595 nm. Cathepsin B activity of all groups was measured. For the assay, 20 microliters were taken, from each group, and the normalization of the results, according to the protein amount was performed.

Measurement were performed using Cathepsin B Activity Assay Kit (Fluorometric) (ab65300) by Abcam (Cambridge, UK). Lysates were treated per Kit instructions that is, incubated with Cathepsin B substrate for 1 hour at 37° C. to identify the activity of the lysosomal protein. The calcium, magnesium, zinc and carbonate supplement sources and concentrations are detailed in the tested groups above.

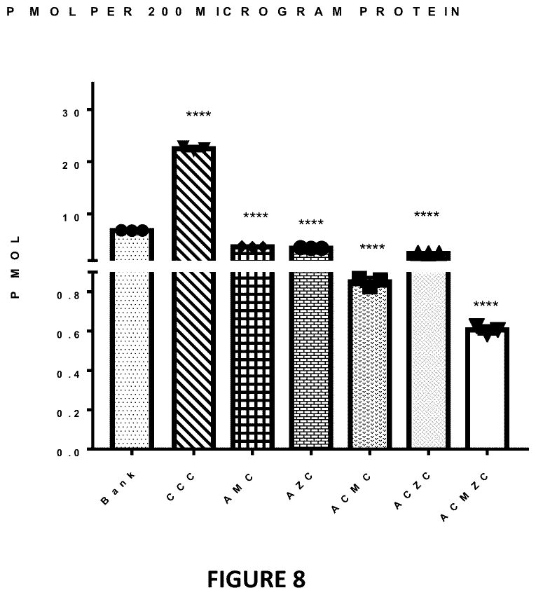

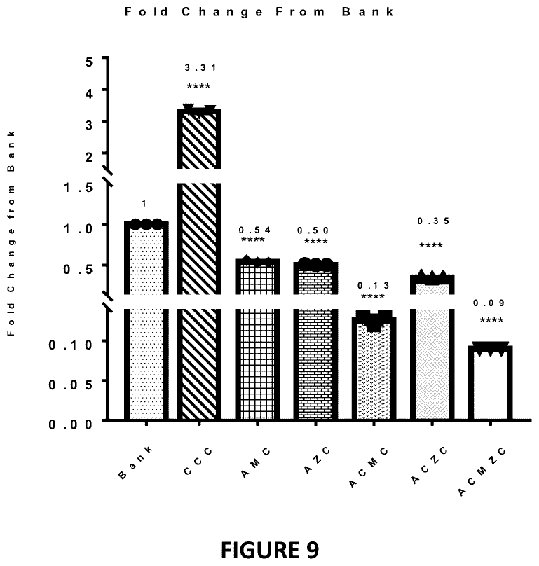

The effects of amorphous metal carbonates on Cathepsin B activity in A549 human lung carcinoma cancer cells were compared to CCC and no treatment. The results were plotted in a graph comparing couples of subcultures with the same additional metals and concentrations with and without the carbonate loads. Data is presented as the mean±SEM (FIGS. 8-9).

### Stock Solutions Preparation

Calcium Chloride Dihydrate—CaCl₂.2H₂O—Volume 1 L. In 0.9 L double distilled water (DDW), weigh 18.35 g CaCl₂) 2H₂O mix well. Complete with DDW to a volume of 1 L.

Sodium Carbonate—Na₂CO₃—Volume 1 L. In 0.9 L DDW, weigh 13.23 g Na₂CO₃ mix well. Complete with DDW to a volume of 1 L.

Penta sodium Triphosphate (TP)—Volume 0.5 L. In 0.4 L DDW, weigh 1.836 g TP mix well. Complete with DDW to a volume of 0.5 L.

Zinc Chloride (ZnCl₂)—Volume 1 L. In 0.9 L DDW, weigh 17.04 g ZnCl₂ mix well. Complete with DDW to a volume of 1 L.

Magnesium Chloride (MgCl₂)—Volume 1 L. In 0.9 L DDW, weigh 25.41 g MgCl₂ mix well. Complete with DDW to a volume of 1 L.

### Suspension and Solution Preparations

The suspensions were prepared from the stock solutions according to the following steps seen in tables 1 to 10 below, in a clean vessel using a sterile magnet stirrer.

TABLE 7
CCC solution preparation from stock solutions

| Step # | Solution | Volume (ml) |
|---|---|---|
| 1 | Calcium Chloride Dehydrate | 4 |
| 2 | DDW | 1 |
| 3 | Sodium Carbonate | 4 |
| 4 | DDW | 1 |

Mixing well until uniform colloidal milky solution is obtained for at least 30 second.

TABLE 8
AMC solution preparation from stock solutions

| Step # | Solution | Volume (ml) |
|---|---|---|
| 1 | Magnesium Chloride Hexahydrate | 4 |
| 2 | TP | 1 |
| 3 | Sodium Carbonate | 4 |
| 4 | TP | 1 |

Mixing well until uniform colloidal milky solution is obtained for at least 30 second.

TABLE 9
AZC solution preparation from stock solutions

| Step # | Solution | Volume (ml) |
|---|---|---|
| 1 | Zinc Chloride | 4 |
| 2 | TP | 1 |
| 3 | Sodium Carbonate | 4 |
| 4 | TP | 1 |

Mixing well until uniform colloidal milky solution is obtained for at least 30 second.

TABLE 10
ACC- AMC (ACMC) solution preparation from stock solutions

| Step # | Solution | Volume (ml) |
|---|---|---|
| 1 | Calcium Chloride Dehydrate | 3.6 |
| 2 | Magnesium Chloride Hexahydrate | 0.4 |
| 3 | TP | 1 |
| 4 | Sodium Carbonate | 4 |
| 5 | TP | 1 |

Mixing well until uniform colloidal milky solution is obtained for at least 30 second.

TABLE 11
ACC - AZC (ACZC) solution preparation from stock solutions

| Step # | Solution | Volume (ml) |
|---|---|---|
| 1 | Calcium Chloride Dehydrate | 3.98 |
| 2 | Zinc Chloride | 0.02 |
| 3 | TP | 1 |
| 4 | Sodium Carbonate | 4 |
| 5 | TP | 1 |

Mixing well until uniform colloidal milky solution is obtained for at least 30 second.

TABLE 12
ACC - AMC -AZC (ACMZC) solution
preparation from stock solutions

| Step # | Solution | Volume (ml) |
|---|---|---|
| 1 | Calcium Chloride Dehydrate | 3.58 |
| 2 | Magnesium Chloride Hexahydrate | 0.4 |
| 3 | Zinc Chloride | 0.02 |
| 4 | TP | 1 |
| | Sodium Carbonate | 4 |
| | TP | 1 |

Mixing well until uniform colloidal milky solution is obtained for at least 30 second.

### Preparation of Nanometric CCC Suspension Formulated as Cell Culture Medium Supplement

Thirty-six (36) ml of 2.0688% Calcium chloride dehydrate solution are mixed with 24 ml of sterile water. Then, 40 ml of 1.299% Sodium carbonate solution are added to precipitate CCC. The obtained CCC suspension, (1 hour after the preparation), is used as a supplement to the culture medium. The suspension is added to the final concentration of 2 mM of calcium ions (40 μl ACC suspension into 1 ml medium).

The results show that amorphous metal carbonate significantly reduced Cathepsin amount and/or activity compared to negative control and CCC. In particular, combinations of 2 or 3 metal carbonates, such as ACMC, ACZC, and ACMZC were shown to be high potency to this extent (FIGS. 8-9).

The inventors are currently performing experimental studies based on commonly known simulated digestion protocol. In brief, the particles of the invention are contacted with an HCl solution at pH 1.5. The particles of the invention (e.g. mixed metal carbonate particles) are weighed prior to contacting thereof with the HCl solution. Subsequently, the concentration of metal cations in the solution phase is estimated (e.g. by ICP), so as to assess the dissolution of the amorphous carbonate particles as indicated by the concentration of metal cations. The particles dissolution should be indicative of the potential bioavailability of the particles including the active agent (e.g. comprising one or more divalent metal cations and/or carbonate anion).

Based on the preliminary data obtained by the inventors, at least partial dissolution of the solid particles has been observed. Furthermore, it has been observed that the pH of the HCl solution increases (from the pH 1.5) and forms a steady state pH.

While certain features of the invention have been described herein, many modifications, substitutions, changes, and equivalents will now occur to those of ordinary skill in the art. It is, therefore, to be understood that the appended claims are intended to cover all such modifications and changes as fall within the true spirit of the invention.

1. A composition comprising a plurality of particles, each of said plurality of particles comprises a first divalent metal, a second divalent metal, and a carbonate specie; said particle has a particle size of between 5 and 500 nm, and at least 70% by total weight of said carbonate specie is amorphous; wherein:
any one of said first divalent metal and said second divalent metal is selected from the group comprising: Ca, Zn, Fe, Cu, Co, Ni, Mn, and Cr; and
a molar ratio between said first divalent metal and said second divalent metal is between 10:0.05 and 1:1.

2. The composition of claim 1, wherein any one of: said particle further comprises a stabilizer; a molar ratio between said stabilizer and said first divalent metal within said particle is between 1:100 and 25:100, said stabilizer is selected from the group consisting of: polyphosphate, phosphorylated amino acid, bisphosphonate, dicarboxylic acid, and any combination thereof.

3.-5. (canceled)

6. The composition of claim 1, further comprising a third metal.

7. The composition of claim 1, wherein any one of: (i) a w/w concentration of said first divalent metal within said particle is between 20 and 55%, and wherein the first divalent metal and the second divalent metal are substantially homogenously distributed within the particle; (ii) at least 90%, or at least 95% by total weight of the carbonate specie within said composition is amorphous; (iii) the first divalent metal is Ca, and said second divalent metal is independently selected from Zn, Fe, Cr, and Mn, or any combination thereof; (iv) optionally the first divalent metal is Zn, and said second divalent metal is independently selected from Ca, Zn, Fe, Cr, Mn, and Mg or any combination thereof (v) said composition is in a form of a powder, and wherein at least 95% by total weight of said carbonate species remain amorphous upon storage of said composition for at least 1 month under appropriate conditions; (vi) a water content of said composition is between 1 and 30% by weight and (vii) any combination of (i) to (vi).

8.-11. (canceled)

12. The composition of claim 2 and a pharmaceutically acceptable liquid carrier, wherein:
said first divalent metal and said second divalent metal is selected from the group consisting of: Ca, Zn, Fe, Cu, Co, Ni, Mg, Mn and Cr; and
a molar ratio between said first divalent metal and said second divalent metal is at least 1%.

13. The composition of claim 12, wherein said liquid carrier is an aqueous solution, and said composition is in a form of a suspension, and optionally wherein at least about 80% by total weight of said carbonate species remain amorphous within said composition for a time period of at least 1 d.

14. (canceled)

15. A composition, comprising a plurality of particles, each of said plurality of particles comprises a divalent metal carbonate including any derivative thereof, and a at least one stabilizing agent, wherein said particle has a diameter of between 5 and 500 nm, and at least 70% by weight of said divalent metal carbonate is amorphous; wherein:
said divalent metal is selected from the group comprising: Zn, Cu, Co, Ni, and Mg; and
a molar ratio between said divalent metal and said at least one stabilizing agent is between 100:1 and 100:25.

16. The composition of claim 15, wherein any one of: (i) if said divalent metal is Mg, then the plurality of particles are characterized by a BET surface area of at most 200 m²/g; (ii) said stabilizer is selected from the group consisting of: polyphosphate, phosphorylated amino acid, bisphosphonate, a dicarboxylic acid, and any combination thereof; (iii) optionally said polyphosphate is selected form the group consisting of: diphosphate, triphosphate, trimetaphosphate, hexametaphosphate, and any combination thereof; (iv) said divalent metal carbonate or a hydrate thereof is Zn or Mg; (v) a weight content of said divalent metal within said particle is between 20 and 60%; (vi) at least 90%, or at least 95% by weight of said divalent metal carbonate within said composition is amorphous; and (vii) any combination of (i) to (vi).

17.-21. (canceled)

22. The composition of claim 15, further comprising a pharmaceutically acceptable carrier, and optionally any one of: (i) said pharmaceutically acceptable carrier is a liquid carrier and wherein said composition is in a form of a suspension; (ii) said suspension is substantially stable for at least 1 h; (iii) at least about 80% by total weight of said divalent metal carbonate remains amorphous within said liquid composition for a time period of at least 1 d; (iv) said composition is in a form of a powder, and wherein at least 95% by total weight of said divalent metal carbonate remains amorphous upon storage of said composition for at least 1 month under appropriate conditions; (v) a water content of said powder is between 1 and 30% by weight and (vi) any combination of (i) to (v).

23.-24. (canceled)

25. The composition of claim 15, and a pharmaceutically acceptable liquid carrier, wherein said divalent metal is Zn or Mg.

26.-28. (canceled)

29. A composition comprising a plurality of particles, each of said plurality of particles comprises a first divalent metal, a second divalent metal, a carbonate specie and a stabilizer; said particle has a particle size of between 5 and 500 nm, and at least 70% by total weight of said carbonate species within said composition are amorphous; wherein:
said first divalent metal is selected from the group consisting of: Ca, Zn, Fe, Cu, Co, Ni, Mn, and Cr;
said second divalent metal is Mg; and
a molar ratio between said first divalent metal and said second divalent metal is between 10:0.0.05 and 10.0:9.9.

30. The composition of claim 29, wherein any one of: (i) a molar ratio between said stabilizer and said first divalent metal within said particle is between 1:100 and 25:100; (ii) said stabilizer is selected form the group consisting of: polyphosphate, phosphoserine, adenosine triphosphate, adenosine diphosphate, phytic acid, citric acid, etidronic acid, pyrophosphate, ethanol, and any combination thereof; (iii) said polyphosphate is selected form the group consisting of: diphosphate, triphosphate, trimetaphosphate, hexametaphosphate, and any combination thereof; (iv) a w/w concentration of said first divalent metal within said particle is between 20 and 55%; (v) a molar ratio between said first divalent metal and Mg is between 10:0.05 and 1:1, or between 10:0.05 and 10:3; and (vi) any combination of (i) to (v).

31.-34. (canceled)

35. The composition of claim 29, further comprising a pharmaceutically acceptable carrier.

36. The composition of claim 35, wherein any one of: (i) said pharmaceutically acceptable carrier is a liquid carrier and wherein said composition is in a form of a suspension; (ii) said suspension is substantially stable for at least 1 h; (iii) at least about 80% by total weight of said carbonate specie remains amorphous within said composition for a time period of at least 1 d; (iv) a water content of said composition is between 1 and 30% by weight (v) said composition is in a form of a powder, and wherein at least 95% by total weight of said carbonate specie remains amorphous upon storage of said composition for at least 1 month under appropriate conditions; and (vi) any combination of (i) to (v).

37.-40. (canceled)

41. The composition of claim 1, wherein said particles are characterized by a BET surface area of between 20 and 200 m²/g, and optionally wherein said divalent metal is in a form of a divalent metal salt, hydroxide, hydroxyl, oxide, a hydrate or any combination thereof.

42. The composition of claim 2, wherein said stabilizer is substantially homogenously distributed within said particle.

43. (canceled)

44. The composition of claim 1, wherein at least a portion of said plurality of particles is in a form of an agglomerate or an aggregate, and optionally wherein said agglomerate or aggregate is characterized by having an average particle size of between 0.1 and 200 μm.

45. (canceled)

46. The composition of claim 1, wherein a molar ratio of the carbonate specie to a total divalent metal content of said particles is between 0.1:1 and 1:1.

47. The composition of claim 1, wherein said carbonate specie is in a form of (i) a metal carbonate of the first divalent metal, (ii) a metal carbonate of the second divalent metal or both (i) and (ii).

48. The composition of claim 1, a pharmaceutical or a nutraceutical composition, and optionally wherein said composition comprises a therapeutically effective amount or a nutraceutically effective amount of said particles.

49. (canceled)
